SEQUENCE LISTING (28 sequences)

SEQ ID NO: 1 - protein, 6 aa, synthetic construct
  REGION 1..6: note = Synthetic
RGDKGE

SEQ ID NO: 2 - protein, 9 aa, synthetic construct
  REGION 1..9: note = Synthetic
KGDRGDAPG

SEQ ID NO: 3 - protein, 9 aa, synthetic construct
  REGION 1..9: note = Synthetic
QGPRGDKGE

SEQ ID NO: 4 - protein, 4 aa, synthetic construct
  REGION 1..4: note = Synthetic
  MOD_RES 3: note = Hydroxyproline
GPPG

SEQ ID NO: 5 - protein, 7 aa, synthetic construct
  REGION 1..7: note = Synthetic
  MOD_RES 3: note = Hydroxyproline
  MOD_RES 6: note = Hydroxyproline
GPPGPPP

SEQ ID NO: 6 - protein, 4 aa, synthetic construct
  REGION 1..4: note = Synthetic
GPPG

SEQ ID NO: 7 - protein, 7 aa, synthetic construct
  REGION 1..7: note = Synthetic
GPPGPPG

SEQ ID NO: 8 - protein, 472 aa, synthetic construct
  REGION 1..472: note = Synthetic: Humanized anti-HU177 heavy chain
MGWTLVFLFLLSVTAGVHSQVTLKESGPALVKPTQTLTLTCTFSGFSLSTPGMGVWWIRQPPGKALEWLADIWWDDNKYTNPSLKSRLTISKDTSKNQVVLTMTNMDPVDTATYYCARRANYGNPYYAQDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 239 aa, synthetic construct
  REGION 1..239: note = Synthetic: Humanized anti-HU177 light chain
MVSSAQFLGLLLLCFQGTRCDIVMTQTPLSLPVTPGEPASISCRSSQSIVSSWGNTYLEWYLQKPGQSPQLLIYKVSNRFSGVPDRFSGSGSGTDFTLKISRVEAEDVGVYYCFQGSHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 1464 aa, synthetic construct
  REGION 1..1464: note = Synthetic: Collagen alpha-1(I)
MFSFVDLRLLLLLAATALLTHGQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 11 - protein, 6 aa, synthetic construct
  REGION 1..6: note = Synthetic
RGDAPG

SEQ ID NO: 12 - protein, 9 aa, synthetic construct
  REGION 1..9: note = Synthetic
AGSRGDGGP

SEQ ID NO: 13 - protein, 9 aa, synthetic construct
  REGION 1..9: note = Synthetic
QGIRGDKGE

SEQ ID NO: 14 - protein, 9 aa, synthetic construct
  REGION 1..9: note = Synthetic
RGPRGDQGP

SEQ ID NO: 15 - protein, 9 aa, synthetic construct
  REGION 1..9: note = Synthetic
QGPSGAPGE

SEQ ID NO: 16 - protein, 8 aa, synthetic construct
  REGION 1..8: note = Synthetic
CPGFPGFC

SEQ ID NO: 17 - protein, 16 aa, synthetic construct
  REGION 1..16: note = Synthetic
PGAKGLPGPPGPPGPY

SEQ ID NO: 18 - protein, 15 aa, synthetic construct
  REGION 1..15: note = Synthetic
  MOD_RES 3: note = Hydroxyproline
  MOD_RES 6: note = Hydroxyproline
  MOD_RES 9: note = Hydroxyproline
  MOD_RES 12: note = Hydroxyproline
  MOD_RES 15: note = Hydroxyproline
GFPGIPGTPGPPGLP

SEQ ID NO: 19 - protein, 15 aa, synthetic construct
  REGION 1..15: note = Synthetic
  MOD_RES 12: note = Hydroxyproline
  MOD_RES 15: note = Hydroxyproline
GEXGDQGIAGFPGSP

SEQ ID NO: 20 - protein, 15 aa, synthetic construct
  REGION 1..15: note = Synthetic
  MOD_RES 9: note = Hydroxyproline
  MOD_RES 12: note = Hydroxyproline
GPQGQPGLPGLPGPM

SEQ ID NO: 21 - protein, 7 aa, synthetic construct
  REGION 1..7: note = Synthetic
  MOD_RES 3: note = Hydroxyproline
  MOD_RES 6: note = Hydroxyproline
GFPGIPT

SEQ ID NO: 22 - protein, 11 aa, synthetic construct
  REGION 1..11: note = Synthetic
  MOD_RES 6: note = Hydroxyproline
  MOD_RES 9: note = Hydroxyproline
GDTGPPGPPGY

SEQ ID NO: 23 - protein, 1366 aa, synthetic construct
  REGION 1..1366: note = Synthetic: Collagen alpha-2(I)
MLSFVDTRTLLLLAVTLCLATCQSLQEETVRKGPAGDRGPRGERGPPGPPGRDGEDGPTGPPGPPGPPGPPGLGGNFAAQYDGKGVGLGPGPMGLMGPRGPPGAAGAPGPQGFQGPAGEPGEPGQTGPAGARGPAGPPGKAGEDGHPGKPGRPGERGVVGPQGARGFPGTPGLPGFKGIRGHNGLDGLKGQPGAPGVKGEPGAPGENGTPGQTGARGLPGERGRVGAPGPAGARGSDGSVGPVGPAGPIGSAGPPGFPGAPGPKGEIGAVGNAGPAGPAGPRGEVGLPGLSGPVGPPGNPGANGLTGAKGAAGLPGVAGAPGLPGPRGIPGPVGAAGATGARGLVGEPGPAGSKGESGNKGEPGSAGPQGPPGPSGEEGKRGPNGEAGSAGPPGPPGLRGSPGSRGLPGADGRAGVMGPPGSRGASGPAGVRGPNGDAGRPGEPGLMGPRGLPGSPGNIGPAGKEGPVGLPGIDGRPGPIGPAGARGEPGNIGFPGPKGPTGDPGKNGDKGHAGLAGARGAPGPDGNNGAQGPPGPQGVQGGKGEQGPPGPPGFQGLPGPSGPAGEVGKPGERGLHGEFGLPGPAGPRGERGPPGESGAAGPTGPIGSRGPSGPPGPDGNKGEPGVVGAVGTAGPSGPSGLPGERGAAGIPGGKGEKGEPGLRGEIGNPGRDGARGAPGAVGAPGPAGATGDRGEAGAAGPAGPAGPRGSPGERGEVGPAGPNGFAGPAGAAGQPGAKGERGAKGPKGENGVVGPTGPVGAAGPAGPNGPPGPAGSRGDGGPPGMTGFPGAAGRTGPPGPSGISGPPGPPGPAGKEGLRGPRGDQGPVGRTGEVGAVGPPGFAGEKGPSGEAGTAGPPGTPGPQGLLGAPGILGLPGSRGERGLPGVAGAVGEPGPLGIAGPPGARGPPGAVGSPGVNGAPGEAGRDGNPGNDGPPGRDGQPGHKGERGYPGNIGPVGAAGAPGPHGPVGPAGKHGNRGETGPSGPVGPAGAVGPRGPSGPQGIRGDKGEPGEKGPRGLPGLKGHNGLQGLPGIAGHHGDQGAPGSVGPAGPRGPAGPSGPAGKDGRTGHPGTVGPAGIRGPQGHQGPAGPPGPPGPPGPPGVSGGGYDFGYDGDFYRADQPRSAPSLRPKDYEVDATLKSLNNQIETLLTPEGSRKNPARTCRDLRLSHPEWSSGYYWIDPNQGCTMDAIKVYCDFSTGETCIRAQPENIPAKNWYRSSKDKKHVWLGETINAGSQFEYNVEGVTSKEMATQLAFMRLLANYASQNITYHCKNSIAYMDEETGNLKKAVILQGSNDVELVAEGNSRFTYTVLVDGCSKKTNEWGKTIIEYKTNKPSRLPFLDIAPLDIGGADQEFFVDIGPVCFK

SEQ ID NO: 24 - protein, 1487 aa, synthetic construct
  REGION 1..1487: note = Synthetic: Collagen alpha-1(II)
MIRLGAPQTLVLLTLLVAAVLRCQGQDVQEAGSCVQDGQRYNDKDVWKPEPCRICVCDTGTVLCDDIICEDVKDCLSPEIPFGECCPICPTDLATASGQPGPKGQKGEPGDIKDIVGPKGPPGPQGPAGEQGPRGDRGDKGEKGAPGPRGRDGEPGTPGNPGPPGPPGPPGPPGLGGNFAAQMAGGFDEKAGGAQLGVMQGPMGPMGPRGPPGPAGAPGPQGFQGNPGEPGEPGVSGPMGPRGPPGPPGKPGDDGEAGKPGKAGERGPPGPQGARGFPGTPGLPGVKGHRGYPGLDGAKGEAGAPGVKGESGSPGENGSPGPMGPRGLPGERGRTGPAGAAGARGNDGQPGPAGPPGPVGPAGGPGFPGAPGAKGEAGPTGARGPEGAQGPRGEPGTPGSPGPAGASGNPGTDGIPGAKGSAGAPGIAGAPGFPGPRGPPGPQGATGPLGPKGQTGEPGIAGFKGEQGPKGEPGPAGPQGAPGPAGEEGKRGARGEPGGVGPIGPPGERGAPGNRGFPGQDGLAGPKGAPGERGPSGLAGPKGANGDPGRPGEPGLPGARGLTGRPGDAGPQGKVGPSGAPGEDGRPGPPGPQGARGQPGVMGFPGPKGANGEPGKAGEKGLPGAPGLRGLPGKDGETGAAGPPGPAGPAGERGEQGAPGPSGFQGLPGPPGPPGEGGKPGDQGVPGEAGAPGLVGPRGERGFPGERGSPGAQGLQGPRGLPGTPGTDGPKGASGPAGPPGAQGPPGLQGMPGERGAAGIAGPKGDRGDVGEKGPEGAPGKDGGRGLTGPIGPPGPAGANGEKGEVGPPGPAGSAGARGAPGERGETGPPGPAGFAGPPGADGQPGAKGEQGEAGQKGDAGAPGPQGPSGAPGPQGPTGVTGPKGARGAQGPPGATGFPGAAGRVGPPGSNGNPGPPGPPGPSGKDGPKGARGDSGPPGRAGEPGLQGPAGPPGEKGEPGDDGPSGAEGPPGPQGLAGQRGIVGLPGQRGERGFPGLPGPSGEPGKQGAPGASGDRGPPGPVGPPGLTGPAGEPGREGSPGADGPPGRDGAAGVKGDRGETGAVGAPGAPGPPGSPGPAGPTGKQGDRGEAGAQGPMGPSGPAGARGIQGPQGPRGDKGEAGEPGERGLKGHRGFTGLQGLPGPPGPSGDQGASGPAGPSGPRGPPGPVGPSGKDGANGIPGPIGPPGPRGRSGETGPAGPPGNPGPPGPPGPPGPGIDMSAFAGLGPREKGPDPLQYMRADQAAGGLRQHDAEVDATLKSLNNQIESIRSPEGSRKNPARTCRDLKLCHPEWKSGDYWIDPNQGCTLDAMKVFCNMETGETCVYPNPANVPKKNWWSSKSKEKKHIWFGETINGGFHFSYGDDNLAPNTANVQMTFLRLLSTEGSQNITYHCKNSIAYLDEAAGNLKKALLIQGSNDVEIRAEGNSRFTYTALKDGCTKHTGKWGKTVIEYRSQKTSRLPIIDIAPMDIGGPEQEFGVDIGPVCFL

SEQ ID NO: 25 - protein, 1466 aa, synthetic construct
  REGION 1..1466: note = Synthetic: Collagen alpha-1(III)
MMSFVQKGSWLLLALLHPTIILAQQEAVEGGCSHLGQSYADRDVWKPEPCQICVCDSGSVLCDDIICDDQELDCPNPEIPFGECCAVCPQPPTAPTRPPNGQGPQGPKGDPGPPGIPGRNGDPGIPGQPGSPGSPGPPGICESCPTGPQNYSPQYDSYDVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPGLAGAPGLRGGAGPPGPEGGKGAAGPPGPPGAAGTPGLQGMPGERGGLGSPGPKGDKGEPGGPGADGVPGKDGPRGPTGPIGPPGPAGQPGDKGEGGAPGLPGIAGPRGSPGERGETGPPGPAGFPGAPGQNGEPGGKGERGAPGEKGEGGPPGVAGPPGGSGPAGPPGPQGVKGERGSPGGPGAAGFPGARGLPGPPGSNGNPGPPGPSGSPGKDGPPGPAGNTGAPGSPGVSGPKGDAGQPGEKGSPGAQGPPGAPGPLGIAGITGARGLAGPPGMPGPRGSPGPQGVKGESGKPGANGLSGERGPPGPQGLPGLAGTAGEPGRDGNPGSDGLPGRDGSPGGKGDRGENGSPGAPGAPGHPGPPGPVGPAGKSGDRGESGPAGPAGAPGPAGSRGAPGPQGPRGDKGETGERGAAGIKGHRGFPGNPGAPGSPGPAGQQGAIGSPGPAGPRGPVGPSGPPGKDGTSGHPGPIGPPGPRGNRGERGSEGSPGHPGQPGPPGPPGAPGPCCGGVGAAAIAGIGGEKAGGFAPYYGDEPMDFKINTDEIMTSLKSVNGQIESLISPDGSRKNPARNCRDLKFCHPELKSGEYWVDPNQGCKLDAIKVFCNMETGETCISANPLNVPRKHWWTDSSAEKKHVWFGESMDGGFQFSYGNPELPEDVLDVHLAFLRLLSSRASQNITYHCKNSIAYMDQASGNVKKALKLMGSNEGEFKAEGNSKFTYTVLEDGCTKHTGEWSKTVFEYRTRKAVRLPIVDIAPYDIGGPDQEFGVDVGPVCFL

SEQ ID NO: 26 - protein, 1669 aa, synthetic construct
  REGION 1..1669: note = Synthetic: Collagen alpha-1(IV)
MGPRLSVWLLLLPAALLLHEEHSRAAAKGGCAGSGCGKCDCHGVKGQKGERGLPGLQGVIGFPGMQGPEGPQGPPGQKGDTGEPGLPGTKGTRGPPGASGYPGNPGLPGIPGQDGPPGPPGIPGCNGTKGERGPLGPPGLPGFAGNPGPPGLPGMKGDPGEILGHVPGMLLKGERGFPGIPGTPGPPGLPGLQGPVGPPGFTGPPGPPGPPGPPGEKGQMGLSFQGPKGDKGDQGVSGPPGVPGQAQVQEKGDFATKGEKGQKGEPGFQGMPGVGEKGEPGKPGPRGKPGKDGDKGEKGSPGFPGEPGYPGLIGRQGPQGEKGEAGPPGPPGIVIGTGPLGEKGERGYPGTPGPRGEPGPKGFPGLPGQPGPPGLPVPGQAGAPGFPGERGEKGDRGFPGTSLPGPSGRDGLPGPPGSPGPPGQPGYTNGIVECQPGPPGDQGPPGIPGQPGFIGEIGEKGQKGESCLICDIDGYRGPPGPQGPPGEIGFPGQPGAKGDRGLPGRDGVAGVPGPQGTPGLIGQPGAKGEPGEFYFDLRLKGDKGDPGFPGQPGMPGRAGSPGRDGHPGLPGPKGSPGSVGLKGERGPPGGVGFPGSRGDTGPPGPPGYGPAGPIGDKGQAGFPGGPGSPGLPGPKGEPGKIVPLPGPPGAEGLPGSPGFPGPQGDRGFPGTPGRPGLPGEKGAVGQPGIGFPGPPGPKGVDGLPGDMGPPGTPGRPGFNGLPGNPGVQGQKGEPGVGLPGLKGLPGLPGIPGTPGEKGSIGVPGVPGEHGAIGPPGLQGIRGEPGPPGLPGSVGSPGVPGIGPPGARGPPGGQGPPGLSGPPGIKGEKGFPGFPGLDMPGPKGDKGAQGLPGITGQSGLPGLPGQQGAPGIPGFPGSKGEMGVMGTPGQPGSPGPVGAPGLPGEKGDHGFPGSSGPRGDPGLKGDKGDVGLPGKPGSMDKVDMGSMKGQKGDQGEKGQIGPIGEKGSRGDPGTPGVPGKDGQAGQPGQPGPKGDPGISGTPGAPGLPGPKGSVGGMGLPGTPGEKGVPGIPGPQGSPGLPGDKGAKGEKGQAGPPGIGIPGLRGEKGDQGIAGFPGSPGEKGEKGSIGIPGMPGSPGLKGSPGSVGYPGSPGLPGEKGDKGLPGLDGIPGVKGEAGLPGTPGPTGPAGQKGEPGSDGIPGSAGEKGEPGLPGRGFPGFPGAKGDKGSKGEVGFPGLAGSPGIPGSKGEQGFMGPPGPQGQPGLPGSPGHATEGPKGDRGPQGQPGLPGLPGPMGPPGLPGIDGVKGDKGNPGWPGAPGVPGPKGDPGFQGMPGIGGSPGITGSKGDMGPPGVPGFQGPKGLPGLQGIKGDQGDQGVPGAKGLPGPPGPPGPYDIIKGEPGLPGPEGPPGLKGLQGLPGPKGQQGVTGLVGIPGPPGIPGFDGAPGQKGEMGPAGPTGPRGFPGPPGPDGLPGSMGPPGTPSVDHGFLVTRHSQTIDDPQCPSGTKILYHGYSLLYVQGNERAHGQDLGTAGSCLRKFSTMPFLFCNINNVCNFASRNDYSYWLSTPEPMPMSMAPITGENIRPFISRCAVCEAPAMVMAVHSQTIQIPPCPSGWSSLWIGYSFVMHTSAGAEGSGQALASPGSCLEEFRSAPFIECHGRGTCNYYANAYSFWLATIERSEMFKKPTPSTLKAGELRTHVSRCQVCMRRT

SEQ ID NO: 27 - protein, 1712 aa, synthetic construct
  REGION 1..1712: note = Synthetic: Collagen alpha-2(IV)
MGRDQRAVAGPALRRWLLLGTVTVGFLAQSVLAGVKKFDVPCGGRDCSGGCQCYPEKGGRGQPGPVGPQGYNGPPGLQGFPGLQGRKGDKGERGAPGVTGPKGDVGARGVSGFPGADGIPGHPGQGGPRGRPGYDGCNGTQGDSGPQGPPGSEGFTGPPGPQGPKGQKGEPYALPKEERDRYRGEPGEPGLVGFQGPPGRPGHVGQMGPVGAPGRPGPPGPPGPKGQQGNRGLGFYGVKGEKGDVGQPGPNGIPSDTLHPIIAPTGVTFHPDQYKGEKGSEGEPGIRGISLKGEEGIMGFPGLRGYPGLSGEKGSPGQKGSRGLDGYQGPDGPRGPKGEAGDPGPPGLPAYSPHPSLAKGARGDPGFPGAQGEPGSQGEPGDPGLPGPPGLSIGDGDQRRGLPGEMGPKGFIGDPGIPALYGGPPGPDGKRGPPGPPGLPGPPGPDGFLFGLKGAKGRAGFPGLPGSPGARGPKGWKGDAGECRCTEGDEAIKGLPGLPGPKGFAGINGEPGRKGDRGDPGQHGLPGFPGLKGVPGNIGAPGPKGAKGDSRTITTKGERGQPGVPGVPGMKGDDGSPGRDGLDGFPGLPGPPGDGIKGPPGDPGYPGIPGTKGTPGEMGPPGLGLPGLKGQRGFPGDAGLPGPPGFLGPPGPAGTPGQIDCDTDVKRAVGGDRQEAIQPGCIGGPKGLPGLPGPPGPTGAKGLRGIPGFAGADGGPGPRGLPGDAGREGFPGPPGFIGPRGSKGAVGLPGPDGSPGPIGLPGPDGPPGERGLPGEVLGAQPGPRGDAGVPGQPGLKGLPGDRGPPGFRGSQGMPGMPGLKGQPGLPGPSGQPGLYGPPGLHGFPGAPGQEGPLGLPGIPGREGLPGDRGDPGDTGAPGPVGMKGLSGDRGDAGFTGEQGHPGSPGFKGIDGMPGTPGLKGDRGSPGMDGFQGMPGLKGRPGFPGSKGEAGFFGIPGLKGLAGEPGFKGSRGDPGPPGPPPVILPGMKDIKGEKGDEGPMGLKGYLGAKGIQGMPGIPGLSGIPGLPGRPGHIKGVKGDIGVPGIPGLPGFPGVAGPPGITGFPGFIGSRGDKGAPGRAGLYGEIGATGDFGDIGDTINLPGRPGLKGERGTTGIPGLKGFFGEKGTEGDIGFPGITGVTGVQGPPGLKGQTGFPGLTGPPGSQGELGRIGLPGGKGDDGWPGAPGLPGFPGLRGIRGLHGLPGTKGFPGSPGSDIHGDPGFPGPPGERGDPGEANTLPGPVGVPGQKGDQGAPGERGPPGSPGLQGFPGITPPSNISGAPGDKGAPGIFGLKGYRGPPGPPGSAALPGSKGDTGNPGAPGTPGTKGWAGDSGPQGRPGVFGLPGEKGPRGEQGFMGNTGPTGAVGDRGPKGPKGDPGFPGAPGTVGAPGIAGIPQKIAVQPGTVGPQGRRGPPGAPGEMGPQGPPGEPGFRGAPGKAGPQGRGGVSAVPGFRGDEGPIGHQGPIGQEGAPGRPGSPGLPGMPGRSVSIGYLLVKHSQTDQEPMCPVGMNKLWSGYSLLYFEGQEKAHNQDLGLAGSCLARFSTMPFLYCNPGDVCYYASRNDKSYWLSTTAPLPMMPVAEDEIKPYISRCSVCEAPAIAIAVHSQDVSIPHCPAGWRSLWIGYSFLMHTAAGDEGGGQSLVSPGSCLEDFRATPFIECNGGRGTCHYYANKYSFWLTTIPEQSFQGSPSADTLKAGLIRTHISRCQVCMKNL

SEQ ID NO: 28 - protein, 1838 aa, synthetic construct
  REGION 1..1838: note = Synthetic: Collagen alpha-1(V)
MDVHTRWKARSALRPGAPLLPPLLLLLLWAPPPSRAAQPADLLKVLDFHNLPDGITKTTGFCATRRSSKGPDVAYRVTKDAQLSAPTKQLYPASAFPEDFSILTTVKAKKGSQAFLVSIYNEQGIQQIGLELGRSPVFLYEDHTGKPGPEDYPLFRGINLSDGKWHRIALSVHKKNVTLILDCKKKTTKFLDRSDHPMIDINGIIVFGTRILDEEVFEGDIQQLLFVSDHRAAYDYCEHYSPDCDTAVPDTPQSQDPNPDEYYTEGDGEGETYYYEYPYYEDPEDLGKEPTPSKKPVEAAKETTEVPEELTPTPTEAAPMPETSEGAGKEEDVGIGDYDYVPSEDYYTPSPYDDLTYGEGEENPDQPTDPGAGAEIPTSTADTSNSSNPAPPPGEGADDLEGEFTEETIRNLDENYYDPYYDPTSSPSEIGPGMPANQDTIYEGIGGPRGEKGQKGEPAIIEPGMLIEGPPGPEGPAGLPGPPGTMGPTGQVGDPGERGPPGRPGLPGADGLPGPPGTMLMLPFRFGGGGDAGSKGPMVSAQESQAQAILQQARLALRGPAGPMGLTGRPGPVGPPGSGGLKGEPGDVGPQGPRGVQGPPGPAGKPGRRGRAGSDGARGMPGQTGPKGDRGFDGLAGLPGEKGHRGDPGPSGPPGPPGDDGERGDDGEVGPRGLPGEPGPRGLLGPKGPPGPPGPPGVTGMDGQPGPKGNVGPQGEPGPPGQQGNPGAQGLPGPQGAIGPPGEKGPLGKPGLPGMPGADGPPGHPGKEGPPGEKGGQGPPGPQGPIGYPGPRGVKGADGIRGLKGTKGEKGEDGFPGFKGDMGIKGDRGEIGPPGPRGEDGPEGPKGRGGPNGDPGPLGPPGEKGKLGVPGLPGYPGRQGPKGSIGFPGFPGANGEKGGRGTPGKPGPRGQRGPTGPRGERGPRGITGKPGPKGNSGGDGPAGPPGERGPNGPQGPTGFPGPKGPPGPPGKDGLPGHPGQRGETGFQGKTGPPGPPGVVGPQGPTGETGPMGERGHPGPPGPPGEQGLPGLAGKEGTKGDPGPAGLPGKDGPPGLRGFPGDRGLPGPVGALGLKGNEGPPGPPGPAGSPGERGPAGAAGPIGIPGRPGPQGPPGPAGEKGAPGEKGPQGPAGRDGLQGPVGLPGPAGPVGPPGEDGDKGEIGEPGQKGSKGDKGEQGPPGPTGPQGPIGQPGPSGADGEPGPRGQQGLFGQKGDEGPRGFPGPPGPVGLQGLPGPPGEKGETGDVGQMGPPGPPGPRGPSGAPGADGPQGPPGGIGNPGAVGEKGEPGEAGEPGLPGEGGPPGPKGERGEKGESGPSGAAGPPGPKGPPGDDGPKGSPGPVGFPGDPGPPGEPGPAGQDGPPGDKGDDGEPGQTGSPGPTGEPGPSGPPGKRGPPGPAGPEGRQGEKGAKGEAGLEGPPGKTGPIGPQGAPGKPGPDGLRGIPGPVGEQGLPGSPGPDGPPGPMGPPGLPGLKGDSGPKGEKGHPGLIGLIGPPGEQGEKGDRGLPGPQGSSGPKGEQGITGPSGPIGPPGPPGLPGPPGPKGAKGSSGPTGPKGEAGHPGPPGPPGPPGEVIQPLPIQASRTRRNIDASQLLDDGNGENYVDYADGMEEIFGSLNSLKLEIEQMKRPLGTQQNPARTCKDLQLCHPDFPDGEYWVDPNQGCSRDSFKVYCNFTAGGSTCVFPDKKSEGARITSWPKENPGSWFSEFKRGKLLSYVDAEGNPVGVVQMTFLRLLSASAHQNVTYHCYQSVAWQDAATGSYDKALRFLGSNDEEMSYDNNPYIRALVDGCATKKGYQKTVLEIDTPKVEQVPIVDIMFNDFGEASQKFGFEVGPACFMG

(19) United States
(12) Patent Application Publication
(10) Pub. No.: US 2024/0358850 A1
(43) Pub. Date: Oct. 31, 2024

(54) COMPOSITIONS AND METHODS FOR AN ANTIBODY DRUG CONJUGATE DIRECTED TO A NON-CELL SURFACE THERAPEUTIC TARGET

(71) Applicant: MaineHealth, Portland, ME (US)

(72) Inventors: Peter C. Brooks, Harpswell, ME (US); Jennifer M. Caron, Scarborough, ME (US)

(21) Appl. No.: 18/633,864

(22) Filed: Apr. 12, 2024

Related U.S. Application Data
(60) Provisional application No. 63/459,013, filed on Apr. 13, 2023.

(51) Int. Cl. A61K 47/68; A61K 39/395; A61P 35/00

(52) U.S. Cl. CPC A61K 47/6843; A61K 39/3955; A61K 47/6803; A61K 47/68031; A61K 47/68033; A61K 47/68035; A61K 47/6809; A61P 35/00

(57) ABSTRACT

The present invention provides compositions and methods of use thereof in the treatment of cancer and abnormal immune suppression diseases with antibodies an antigen binding fragments thereof which bind collagen and antibody drug conjugates (ADC). In some embodiments, in the methods provided herein the ADCs target non-cell surface targets associated with tumors.

### RELATED APPLICATION

The instant application claims priority to U.S. Provisional Application No. 63/459,013, filed Apr. 13, 2023, the entire contents of which are expressly incorporated by reference herein.

### SEQUENCE LISTING

The application contains a Sequence Listing which has been submitted electronically in .XML format and is hereby incorporated by reference in its entirety. Said. XML copy, created on Apr. 11, 2024, is named “136191-00302.xml” and is 53,251 bytes in size. The sequence listing contained in this .XML file is part of the specification and is hereby incorporated by reference herein in its entirety.

### BACKGROUND OF THE INVENTION

Despite progress on some fronts, the treatment of cancer is still critically lacking in general effectiveness and continues to rely primarily on surgery and chemotherapy. Accordingly, the present invention addresses this and other needs.

### SUMMARY OF THE INVENTION

Accordingly, in one aspect, the present invention provides an antibody-drug conjugate (ADC) comprising an antibody, or an antigen binding portion thereof, conjugated to at least one drug, and wherein the antibody, or the antigen binding fragment thereof, binds to collagen and is internalized in a cancer cell. In one embodiment, the collagen comprises collagen type-I, collagen type II, collagen type III, or collagen type-IV. In one embodiment, the collagen is collagen type-I. In one embodiment, the collagen is denatured.

In one embodiment, the antibody, or the antigen binding fragment thereof binds a cryptic collagen epitope comprising one or more amino acid sequences selected from the group consisting of GPOG (SEQ ID NO: 4), GPOGPOP (SEQ ID NO: 5), GPPG (SEQ ID NO: 6), and GPPGPPG (SEQ ID NO: 7). In one embodiment, the antibody, or the antigen binding fragment thereof binds a cryptic collagen epitope comprising one or more amino acid sequences selected from the group consisting of CPGFPGFC (SEQ ID NO: 16), PGAKGLPGPPGPPGPY (SEQ ID NO: 17), GFOGIOGTOGPOGLO (SEQ ID NO: 18), GEXGDQGIAGFOGSO (SEQ ID NO: 19), GPQGQPGLOGLOGPM (SEQ ID NO: 20), GFOGIOT (SEQ ID NO: 21), and GDTGPOGPOGY (SEQ ID NO: 22). In one embodiment, the antibody, or the antigen binding fragment thereof binds a cryptic collagen epitope comprising an amino acid sequence that has at least 90% identity to PGAKGLPGPPGPPGPY (SEQ ID NO: 17).

In one embodiment, the antibody comprises a monoclonal antibody. In one embodiment, the monoclonal antibody comprises an HU177 monoclonal antibody deposited with ATCC accession number PTA-6551.

In one embodiment, the ADC is not directed to a receptor on the cancer cell surface.

In one embodiment, the at least one drug is selected from the group consisting of an anti-apoptotic agent, a mitotic inhibitor, an anti-tumor antibiotic, an immunomodulating agent, a nucleic acid for gene therapy, an anti-angiogenic agent, an anti-metabolite, a boron-containing agent, a chemoprotective agent, a hormone agent, an anti-hormone agent, a DNA damaging agent, a corticosteroid, a photoactive therapeutic agent, an oligonucleotide, a radionuclide agent, a radiosensitizer, a topoisomerase inhibitor, and a tyrosine kinase inhibitor. In one embodiment, the mitotic inhibitor is selected from the group consisting of monomethyl auristatin E (MMAE), monomethyl auristatin F(MMAF), mertansine (DM1), and ravtansine (DM4). In one embodiment, the mitotic inhibitor is monomethyl auristatin E (MMAE). In one embodiment, the mitotic inhibitor is monomethyl auristatin F(MMAF). In one embodiment, the DNA damaging agent is selected from the group consisting of psilocybin, gliclazomycin, streptomycin, pyrrolobenzodiazepine (PBD), doxorubicin, and adrianmycin.

In one embodiment, the at least one drug is conjugated to the antibody, or the antigen-binding portion thereof, via a linker. In one embodiment, the linker is a cleavable linker or a non-cleavable linker.

In another aspect, the present invention provides a pharmaceutical composition comprising the ADC as provided herein, and a pharmaceutically acceptable carrier.

In another aspect, the present invention provides a method of treating cancer in a subject, the method comprising administering to the subject a therapeutically effective amount of the ADC as provided herein, or the pharmaceutical composition as provided herein, thereby treating the cancer.

In another aspect, the present invention provides a method for inhibiting or decreasing growth of a solid tumor in a subject having a solid tumor, the method comprising administering to the subject a therapeutically effective amount of the ADC as provided herein, or the pharmaceutical composition as provided herein, thereby inhibiting or decreasing the growth of the solid tumor in the subject.

In one embodiment, the mean tumor volume in the subject who has been administered the ADC is less than or equal to a control level. In one embodiment, the growth rate of the tumor in the subject who has been administered the ADC is less than or equal to a control level. In one embodiment, the mean tumor volume in the subject who has been administered the ADC is at least 1.1 fold, at least 1.5 fold, at least 2 fold, at least 5 fold, at least 10 fold, at least 20 fold, at least 50 fold, at least 100 fold, at least 200 fold lower compared to a control. In one embodiment, the growth rate of the tumor in the subject who has been administered the ADC is at least 1.1 fold, at least 1.5 fold, at least 2 fold, at least 5 fold, at least 10 fold, at least 20 fold, at least 50 fold, at least 100 fold, at least 200 fold lower compared to a control.

In one embodiment, the ADC is cytotoxic to tumor cells. In one embodiment, the administration of the ADC induces cell death of the tumor cells. In one embodiment, cell death in the tumor in the subject who has been administered the ADC is at least 1.1 fold, at least 1.5 fold, at least 2 fold, at least 5 fold, at least 10 fold, at least 20 fold, at least 50 fold, at least 100 fold, at least 200 fold higher compared to a control.

In one embodiment, the control is prior to administration of the ADC.

In another aspect, the present invention provides a method of delivering a drug to a cancer cell in a subject, the method comprising administering to the subject a therapeutically effective amount of the ADC as provided herein, or the pharmaceutical composition as provided herein. In one embodiment, the ADC is cytotoxic to the cancer cell. In one embodiment, the administration of the ADC induces cell death of the cancer cell.

In one embodiment, the methods as provided herein, further comprise administering concurrent or sequential radiotherapy, monoclonal antibodies, chemotherapy, immunotherapy or other anticancer drugs or interventions to the subject. In one embodiment, the immunotherapy comprises administering an immune checkpoint inhibitor. In one embodiment, the immune checkpoint inhibitor comprises an inhibitor of CTLA-4, PD-1, PDL-1, Lag3, LAIR1, or LAIR2. In one embodiment, the immune checkpoint inhibitor comprises an anti-CTLA-4 antibody, an anti-PD-1 antibody, an anti-PDL-1 antibody, an anti-Lag3 antibody, an anti-LAIR1 antibody, or an anti-LAIR 2 antibody. In one embodiment, the immune checkpoint inhibitor comprises an anti-PD-1 antibody, or an anti-PDL-1 antibody.

In one embodiment, the methods as provided herein further comprise inhibiting an inflammatory condition, wherein said inflammatory condition is selected from the group consisting of an allergy, ankylosing spondylitis, asthma, atopic dermatitis, an autoimmune disease or disorder, a cancer, celiac disease, chronic obstructive pulmonary disease (COPD), chronic peptic ulcer, cystic fibrosis, diabetes, glomerulonephritis, gout, hepatitis, an immune-mediated disease or disorder, inflammatory bowel disease (IBD), myositis, osteoarthritis, pelvic inflammatory disease (PID), multiple sclerosis, neurodegenerative diseases of aging, a periodontal disease, reperfusion injury transplant rejection, psoriasis, pulmonary fibrosis, rheumatic disease, scleroderma, sinusitis, dermatitis, pneumonitis, colitis and tuberculosis.

In one embodiment, the cancer is selected from the group comprising of melanoma, central nervous system (CNS) cancer, CNS germ cell tumor, lung cancer, leukemia, multiple myeloma, renal cancer, malignant glioma, medulloblastoma, breast cancer, ovarian cancer, prostate cancer, bladder cancer, fibrosarcoma, pancreatic cancer, gastric cancer, head and neck cancer, colorectal cancer, a cancer cell derived from a solid cancer, or hematological cancer. In one embodiment, the hematological cancer is a leukemia or a lymphoma, optionally wherein the leukemia is acute lymphoblastic leukemia (ALL), acute myelogenous leukemia (AML), chronic lymphocytic leukemia (CLL), small lymphocytic lymphoma (SLL), chronic myelogenous leukemia (CML) or acute monocytic leukemia (AMOL). In one embodiment, the lymphoma is follicular lymphoma, Hodgkin's lymphoma, or Non-Hodgkin's lymphoma, optionally wherein the Hodgkin's lymphoma is Nodular sclerosing subtype, mixed-cellularity subtype, lymphocyte-rich subtype, or lymphocyte depleted subtype. In one embodiment, the cancer is ovarian cancer. In one embodiment, the cancer is melanoma. In one embodiment, the cancer is fibrosarcoma. In one embodiment, the cancer is lung cancer. In one embodiment, the cancer is breast cancer. In one embodiment, the cancer is prostate cancer. In one embodiment, the cancer is pancreatic cancer.

In one embodiment, the subject is a human.

In another aspect, the present invention provides an antibody-drug conjugate (ADC) comprising an antibody, or an antigen binding portion thereof, conjugated to at least one drug, and wherein the antibody, or the antigen binding fragment thereof, binds to collagen and is internalized in a cancer cell. In one embodiment, the collagen comprises collagen type-I, collagen type II, collagen type III, or collagen type-IV. In one embodiment, the collagen is collagen type-I. In one embodiment, the collagen is denatured.

In one embodiment, the antibody, or the antigen binding fragment thereof binds a cryptic collagen epitope comprising one or more amino acid sequences selected from the group consisting of RGDKGE (SEQ ID NO: 1), KGDRGDAPG (SEQ ID NO: 2), QGPRGDKGE (SEQ ID NO: 3), AGSRGDGGP (SEQ ID NO: 12), QGIRGDKGE (SEQ ID NO: 13), and RGPRGDQGP (SEQ ID NO: 14). In one embodiment, the antibody, or the antigen binding fragment thereof binds a cryptic collagen epitope comprising one or more amino acid sequences selected from the group consisting of RGDKGE (SEQ ID NO: 1), QGPRGDKGE (SEQ ID NO: 3), and QGIRGDKGE (SEQ ID NO: 13). In one embodiment, the antibody comprises a monoclonal antibody. In one embodiment, the monoclonal antibody comprises an XL313 monoclonal antibody deposited with ATCC accession number PTA-6552.

In one embodiment, the ADC is not directed to a receptor on the cancer cell surface.

In one embodiment, the at least one drug is selected from the group consisting of an anti-apoptotic agent, a mitotic inhibitor, an anti-tumor antibiotic, an immunomodulating agent, a nucleic acid for gene therapy, an anti-angiogenic agent, an anti-metabolite, a boron-containing agent, a chemoprotective agent, a hormone agent, an anti-hormone agent, a DNA damaging agent, a corticosteroid, a photoactive therapeutic agent, an oligonucleotide, a radionuclide agent, a radiosensitizer, a topoisomerase inhibitor, and a tyrosine kinase inhibitor. In one embodiment, the mitotic inhibitor is selected from the group consisting of monomethyl auristatin E (MMAE), monomethyl auristatin F(MMAF), mertansine (DM1), and ravtansine (DM4). In one embodiment, the mitotic inhibitor is monomethyl auristatin E (MMAE). In one embodiment, the mitotic inhibitor is monomethyl auristatin F(MMAF). In one embodiment, the DNA damaging agent is selected from the group consisting of psilocybin, gliclazomycin, streptomycin, pyrrolobenzodiazepine (PBD), doxorubicin, and adrianmycin.

In one embodiment, the at least one drug is conjugated to the antibody, or the antigen-binding portion thereof, via a linker. In one embodiment, the linker is a cleavable linker or a non-cleavable linker.

In another aspect, the present invention provides a pharmaceutical composition comprising the ADC as provided herein, and a pharmaceutically acceptable carrier.

In another aspect, the present invention provides a method of treating cancer in a subject, the method comprising administering to the subject a therapeutically effective amount of the ADC as provided herein, or the pharmaceutical composition as provided herein, thereby treating the cancer.

In another aspect, the present invention provides a method for inhibiting or decreasing growth of a solid tumor in a subject having a solid tumor, the method comprising administering to the subject a therapeutically effective amount of the ADC as provided herein, or the pharmaceutical composition as provided herein, thereby inhibiting or decreasing the growth of the solid tumor in the subject.

In one embodiment, the mean tumor volume in the subject who has been administered the ADC is less than or equal to a control level. In one embodiment, the growth rate of the tumor in the subject who has been administered the ADC is less than or equal to a control level. In one embodiment, the mean tumor volume in the subject who has been administered the ADC is at least 1.1 fold, at least 1.5 fold, at least 2 fold, at least 5 fold, at least 10 fold, at least 20 fold, at least 50 fold, at least 100 fold, at least 200 fold lower compared to a control. In one embodiment, the growth rate of the tumor in the subject who has been administered the ADC is at least 1.1 fold, at least 1.5 fold, at least 2 fold, at least 5 fold, at least 10 fold, at least 20 fold, at least 50 fold, at least 100 fold, at least 200 fold lower compared to a control.

In one embodiment, the ADC is cytotoxic to tumor cells. In one embodiment, the administration of the ADC induces cell death of the tumor cells. In one embodiment, cell death in the tumor in the subject who has been administered the ADC is at least 1.1 fold, at least 1.5 fold, at least 2 fold, at least 5 fold, at least 10 fold, at least 20 fold, at least 50 fold, at least 100 fold, at least 200 fold higher compared to a control.

In one embodiment, the control is prior to administration of the ADC.

In another aspect, the present invention provides a method of delivering a drug to a cancer cell in a subject, the method comprising administering to the subject a therapeutically effective amount of the ADC as provided herein, or the pharmaceutical composition as provided herein. In one embodiment, the ADC is cytotoxic to the cancer cell. In one embodiment, the administration of the ADC induces cell death of the cancer cell.

In one embodiment, the methods as provided herein, further comprise administering concurrent or sequential radiotherapy, monoclonal antibodies, chemotherapy, immunotherapy or other anticancer drugs or interventions to the subject. In one embodiment, the immunotherapy comprises administering an immune checkpoint inhibitor. In one embodiment, the immune checkpoint inhibitor comprises an inhibitor of CTLA-4, PD-1, PDL-1, Lag3, LAIR1, or LAIR2. In one embodiment, the immune checkpoint inhibitor comprises an anti-CTLA-4 antibody, an anti-PD-1 antibody, an anti-PDL-1 antibody, an anti-Lag3 antibody, an anti-LAIR1 antibody, or an anti-LAIR 2 antibody. In one embodiment, the immune checkpoint inhibitor comprises an anti-PD-1 antibody, or an anti-PDL-1 antibody.

In one embodiment, the methods as provided herein further comprise inhibiting an inflammatory condition, wherein said inflammatory condition is selected from the group consisting of an allergy, ankylosing spondylitis, asthma, atopic dermatitis, an autoimmune disease or disorder, a cancer, celiac disease, chronic obstructive pulmonary disease (COPD), chronic peptic ulcer, cystic fibrosis, diabetes, glomerulonephritis, gout, hepatitis, an immune-mediated disease or disorder, inflammatory bowel disease (IBD), myositis, osteoarthritis, pelvic inflammatory disease (PID), multiple sclerosis, neurodegenerative diseases of aging, a periodontal disease, reperfusion injury transplant rejection, psoriasis, pulmonary fibrosis, rheumatic disease, scleroderma, sinusitis, dermatitis, pneumonitis, colitis and tuberculosis.

In one embodiment, the cancer is selected from the group comprising of melanoma, central nervous system (CNS) cancer, CNS germ cell tumor, lung cancer, leukemia, multiple myeloma, renal cancer, malignant glioma, medulloblastoma, breast cancer, ovarian cancer, prostate cancer, bladder cancer, fibrosarcoma, pancreatic cancer, gastric cancer, head and neck cancer, colorectal cancer, a cancer cell derived from a solid cancer, or hematological cancer. In one embodiment, the hematological cancer is a leukemia or a lymphoma, optionally wherein the leukemia is acute lymphoblastic leukemia (ALL), acute myelogenous leukemia (AML), chronic lymphocytic leukemia (CLL), small lymphocytic lymphoma (SLL), chronic myelogenous leukemia (CML) or acute monocytic leukemia (AMOL). In one embodiment, the lymphoma is follicular lymphoma, Hodgkin's lymphoma, or Non-Hodgkin's lymphoma, optionally wherein the Hodgkin's lymphoma is Nodular sclerosing subtype, mixed-cellularity subtype, lymphocyte-rich subtype, or lymphocyte depleted subtype. In one embodiment, the cancer is ovarian cancer. In one embodiment, the cancer is melanoma. In one embodiment, the cancer is fibrosarcoma. In one embodiment, the cancer is lung cancer. In one embodiment, the cancer is breast cancer. In one embodiment, the cancer is prostate cancer. In one embodiment, the cancer is pancreatic cancer.

In one embodiment, the subject is a human.

### BRIEF DESCRIPTION OF THE DRAWINGS

FIGS. 1A-1F. Selective internalization of anti-HU177 Mab in different malignant tumor cell types. Microscope slides were coated with denatured collagen type-IV and then blocked with BSA to limit non-specific binding. The indicated cell types, B16F10 (FIG. 1A), MCA205 (FIG. 1B), primary mouse T-cells (FIG. 1C), ID8 ovarian carcinoma cells (FIG. 1D), SKOV-3 ovarian carcinoma cells (FIG. 1E), SKOV-3 ovarian carcinoma cells (FIG. 1F), were serum starved in suspension then mixed with the indicated human antibodies, anti-HU177 (FIGS. 1A-1F) and Ab control (FIG. 1A-1E) or anti-XL166 (FIG. 1F) pre-labeled with the fluorescent internalization probe (pHrod™) and seeded on the coated slides. The cells were allowed to incubate for 1 to 3 hours. The cells were next washed, fixed and counter stained with DAPI to indicate nuclear contents (Blue). Next the cells were then analyzed by fluorescence microscopy for the presence of the released fluorescent internalization probe (Red color). Photos represent examples of internalization of the antibodies (Red color) under each condition for each different cell type. White arrows indicate examples of cells with internalized antibodies. All photos taken at a magnification of 400×. Scale bar indicates 20 micrometers.

FIGS. 2A-2C. Conjugation of the anti-HU177 antibody to the therapeutic payload MMAE failed to alter its functional binding activity. The humanized anti-HU177 antibody and non-specific human control antibody was conjugated with the cytotoxic drug MMAE by using the commercially available MMAE antibody conjugation kit (Perkit™ Antibody MMAE Conjugation Kit) according to manufacturer's instructions. The unconjugated and MMAE-conjugated antibodies were next tested for their ability to bind denatured collagen type-IV. Briefly, microtiter wells were coated with denatured collagen type-IV. Wells were next washed and blocked with BSA to limit non-specific binding. Wells were next incubated with the indicated antibodies and allowed to bind. Wells were washed and antibody binding was quantified by ELISA. FIG. 2A. Quantification of antibody binding to denatured collagen type-IV using 0.5 μg/ml of each antibody. FIG. 2B. Quantification of antibody binding to denatured collagen type-IV using 1.0 μg/ml of each antibody. FIG. 2C. Data bars indicate mean antibody binding from triplicate wells+SE from each condition. The unconjugated and MMAE-conjugated antibodies were also tested for their ability to inhibit cell adhesion to denatured collagen type-IV. Briefly, non-tissue culture wells were coated with denatured collagen type-IV. Wells were next washed and blocked with BSA to limit non-specific binding. B16F10 melanoma cells were mixed with the indicated antibodies (100 μg/ml) and allowed to bind to the denatured collagen coated wells. FIG. 2C. Example of the effects of each antibody on cell adhesion. Data bars indicate mean cell adhesion+SE from triplicate wells from each condition.

FIGS. 3A-3D. Induction of morphological cell changes within several malignant tumor cell types. Briefly, non-tissue culture wells were coated with denatured collagen type-IV, washed and then blocked with BSA to limit non-specific binding. The indicated tumor cell types were then resuspended with the indicated unconjugated or MMAE-conjugated antibodies at a concentration of 50 μg/ml and seeded on the coated wells. The cells were allowed to incubate over a time course. FIG. 3A. Examples of morphological changes in MCA205 fibrosarcoma cells at 24 hours under each experimental treatment condition. FIG. 3B. Examples of morphological changes in SKOV-3 ovarian carcinoma cells at 48 hours under each experimental treatment condition. FIG. 3C. Examples of morphological changes in B16F10 melanoma cells at 72 hours under each experimental treatment condition. FIG. 3D. Examples of morphological changes in ID8 ovarian carcinoma cells at 72 hours under each experimental treatment condition. Black arrows indicate examples of cells undergoing morphological changes. Photos taken at a magnification of 50×.

FIGS. 4A-4C. The anti-HU177-MMAE ADC inhibits the growth of metastatic B16F10 tumor cells. Briefly, 96-well microtiter wells were coated with denatured collagen type-IV, washed and then blocked with BSA to limit non-specific binding. The B16F10 tumor cells were resuspended in serum containing medium with the indicated unconjugated or MMAE-conjugated antibodies at a concentration of 50 μg/ml and seeded on the coated wells. The cells were allowed to grow over a time course of 72 hours. FIG. 4A. Quantification of mean B16F10 melanoma cell growth from triplicate wells at each time point. FIG. 4B. Quantification of mean B16F10 melanoma cell cytotoxicity induced with 50 μg/ml of each antibody from triplicate wells at 72 hrs. FIG. 4C. Quantification of mean B16F10 melanoma cell cytotoxicity induced with 100 μg/ml of each antibody from triplicate wells at 72 hrs.

FIGS. 5A-5F. The anti-HU177-MMAE ADC significantly inhibits the growth of multiple tumor cell types. Briefly, 96-well microtiter wells were coated with denatured collagen type-IV, washed and then blocked with BSA to limit non-specific binding. The indicated tumor cell types were then resuspended in serum containing medium with the indicated unconjugated or MMAE-conjugated antibodies at a concentration of 50 μg/ml and seeded on the coated wells. The tumor cells were allowed to grow over a time course of 72 hours. FIG. 5A. Quantification of mean MCA205 fibrosarcoma cell growth from triplicate wells at each time point. FIG. 5B. Quantification of mean ID8 ovarian carcinoma cell growth from triplicate wells at each time point. FIG. 5C. Quantification of mean SKOV-3 ovarian carcinoma cell growth from triplicate wells at each time point. FIG. 5D. Quantification of mean MCA205 fibrosarcoma cell cytotoxicity induced with 50 μg/ml of each antibody from triplicate wells at 72 hrs. FIG. 5E. Quantification of mean SKOV-3 ovarian carcinoma cell cytotoxicity induced with 50 μg/ml of each antibody from triplicate wells at 72 hrs. FIG. 5F. Quantification of mean ID8 ovarian carcinoma cell cytotoxicity induced with 50 μg/ml of each antibody from triplicate wells at 72 hrs.

FIGS. 6A-6E. The Anti-HU177-MMAE ADC induces cytotoxicity of multiple human tumor cell types. FIG. 6A. Microtiter wells were coated with denatured collagen type-IV, washed and then blocked with BSA to limit non-specific binding. Serum starved HEYa8 ovarian tumor cells were resuspended in serum (2.0% FBS) containing medium with the indicated unconjugated or MMAE-conjugated antibodies at a concentration of 50 μg/ml and seeded on the coated wells. Cytotoxicity was quantified at 72 hours. Data bars indicate mean cellular cytotoxicity from background subtracted triplicate wells. FIG. 6B. Microtiter wells were coated with denatured collagen type-IV, washed and then blocked with BSA to limit non-specific binding. Serum starved PC3M prostate tumor cells were resuspended in serum (5.0% FBS) containing medium with the indicated unconjugated or MMAE-conjugated antibodies at a concentration of 50 μg/ml and seeded on the coated wells. Cytotoxicity was quantified at 72 hours. Data bars indicate mean cellular cytotoxicity from background subtracted triplicate wells. FIG. 6C. Microtiter wells were coated with denatured collagen type-IV, washed and then blocked with BSA to limit non-specific binding. Scrum starved HUVEC cells were resuspended in serum (2.0% FBS) containing medium with the indicated unconjugated or MMAE-conjugated antibodies at a concentration of 15 μg/ml and seeded on the coated wells. Cytotoxicity was quantified at 72 hours. Data bars indicate mean cellular cytotoxicity from background subtracted triplicate wells. FIG. 6D-6E. Microtiter wells were coated with denatured collagen type-IV, washed and then blocked with BSA to limit non-specific binding. Serum starved Pancreatic tumor cells BxPC3 or Panc-1 were resuspended in serum (5.0% FBS) containing medium with the indicated unconjugated or MMAE-conjugated antibodies at a concentration of 50 μg/ml and seeded on the coated wells. Cytotoxicity was quantified at 72 hours. FIG. 6D. Data bars indicate mean BXPC3 cellular cytotoxicity from background subtracted triplicate wells. FIG. 6E. Data bars indicate mean Panc-1 cellular cytotoxicity from background subtracted triplicate wells.

FIGS. 7A-7B. The Anti-HU177-MMAE ADC induces cytotoxicity of murine tumor cells. FIG. 7A Microtiter wells were coated with denatured collagen type-IV, washed and then blocked with BSA to limit non-specific binding. Serum starved LLC lung tumor cells were resuspended in serum (5.0% FBS) containing medium with the indicated unconjugated or MMAE-conjugated antibodies at a concentration of 50 g/ml and seeded on the coated wells. Cytotoxicity was quantified at 72 hours. Data bars indicate mean cellular cytotoxicity from background subtracted triplicate wells. FIG. 7B. Microtiter wells were coated with denatured collagen type-IV, washed and then blocked with BSA to limit non-specific binding. Scrum starved 4T1 breast tumor cells were resuspended in serum (5.0% FBS) containing medium with the indicated unconjugated or MMAE-conjugated antibodies at a concentration of 75 μg/ml and seeded on the coated wells. Cytotoxicity was quantified at 72 hours. Data bars indicate mean cellular cytotoxicity from background subtracted triplicate wells.

FIG. 8. The Anti-HU177-MMAE ADC enhances the therapeutic activity of Anti-HU177 antibody on ovarian tumors in vivo. Mice (N=6 per condition) were injected subcutaneously with human SKOV-3 tumor cells and allowed to establish pre-existing tumors. Beginning on day 6 (black arrow), mice were treated 3× per week with 100 μg per injection of the indicated unconjugated or MMAE conjugated antibodies. Treatment was stopped on Day 21. Mice were sacrificed on Day 32 and all tumors analyzed. Data points indicate the mean tumor volumes at indicated times (+S.E) from each condition.

FIG. 9. The Anti-HU177-MMAE ADC enhances the therapeutic activity of Anti-HU177 antibody on Fibrosarcoma tumors in vivo. Mice (N=6 per condition) were injected subcutaneously with murine MCA205 tumor cells and allowed to establish pre-existing tumors. Beginning on day 7 (black arrow), mice were treated 3× per week with 100 μg per injection of the indicated unconjugated or MMAE conjugated antibodies. Data points indicate the mean tumor volumes at indicated times (+S.E) from each condition.

FIGS. 10A-10C. An anti-HU177-MMAF ADC induces cytotoxicity of murine tumor cells. Microtiter wells were coated with denatured collagen type-IV, washed and then blocked with BSA to limit non-specific binding. The indicated serum starved tumor cells were resuspended in serum (5.0% FBS) containing medium with the indicated unconjugated or MMAF-conjugated antibodies at a concentration of 50 μg/ml and seeded on the coated wells. Cytotoxicity was quantified at 72 hours. FIG. 10A Data bars indicate mean cellular cytotoxicity (+S.E) for human SKOV-3 ovarian carcinoma cells from background subtracted triplicate wells. FIG. 10B Data bars indicate mean cellular cytotoxicity (+S.E) for human prostate (PC3M) carcinoma cells from background subtracted triplicate wells. FIG. 10C Data bars indicate mean cellular cytotoxicity (+S.E) for murine B16F10 melanoma cells from background subtracted triplicate wells.

### DETAILED DESCRIPTION

It is well accepted that the vascular endothelium of malignant tumors is very different than that of normal blood vessels. In general, tumor vessels exhibit a higher level of cellular proliferation, they are also often characterized as being unstructured, leaky, exhibiting disrupted, often incompletely formed vascular basement membrane networks that underlay the endothelial cells. Vascular basement membranes (BMs) are predominately composed of different isoforms of extracellular matrix (ECM) proteins such as collagen type-IV and laminin that form a network of sheet-like structures that provide a restrictive vascular ECM barrier. Importantly, tumor blood vessels provide a major conduit for a variety of innate immune system cells such as macrophages as well as a variety of different adaptive immune cells, including various subsets of T-cells. Thus, the tumor-associated vasculature represents a critical conduit through which many cell types migrate into and out of the tumor microenvironment in a bi-directional manner. In this regard, the mechanisms that regulate the ability of immune cells to traverse the interconnected ECM barrier of the tumor is not completely understood. While much progress has been made in understanding the molecular mechanism of ameboid cell motility that controls trans-endothelial migration through the endothelial cell layer, much less is known about the mechanisms that control the ability of immune cells to breach and migrate through the restrictive subendothelial basement membrane.

Angiogenesis, the process by which new blood vessels form from pre-existing vessels plays a critical role in normal and pathological events. Efforts are underway to more precisely define the interconnected mechanisms that control this crucial biological process in order to develop more effective strategies to control neovascular diseases. Significant advances have been made in identifying molecular regulators of angiogenesis and their associated signaling pathways. A more precise understanding of angiogenic signaling pathways and the networks of regulatory feedback loops operating within distinct cellular compartments has provided important clues to help explain the modest clinical impact of many anti-angiogenic strategies. For example, while vascular endothelial growth factor (VEGF) induces pro-angiogenic signaling leading to enhanced endothelial cell migration, growth, and survival, VEGF-induced blood vessels are often characterized as immature, unstable, and leaky and can regress in the absence of additional signaling events. VEGF stimulation under specific circumstances may lead to inhibition of angiogenesis in the context of altered PDGF signaling do to disruption of pericyte recruitment. These unexpected findings provide evidence of a negative role for the VEGF/VEGFR signaling during new vessel development. Similarly, studies have provided evidence for both a positive and a negative role for integrin avβ3 in angiogenesis.

A wide array of alterations in the composition and biomechanical properties of extracellular matrix (ECM) proteins are known to occur during angiogenesis and studies are beginning to define how these changes contribute to new blood vessel development. Among the key cell surface molecules that play roles in mechano-transduction to facilitate information flow from outside the cell to the inside are integrin receptors. Integrins may act like information hubs by sensing diverse extracellular inputs and relaying this information into a complex network of intracellular circuits that ultimately modulate cellular behavior. The precise molecular mechanisms by which cells fine-tune their response to changes within the stromal microenvironment are not completely understood. Further complicating the understanding of new vessel development is the expanding number of cell types that contribute to tissue specific control of angiogenesis such as distinct subsets of stromal fibroblasts, progenitor cells and a variety of inflammatory cells such as neutrophils, mast cells and macrophages. The roles played by these diverse cells during angiogenesis range from secretion of cytokines, chemokines and proteolytic enzymes to the differential expression of other pro and anti-angiogenic factors. Thus, tight control mechanisms must operate to allow coordination between these diverse compartments to govern tissue specific vascular responses.

Integrins are molecules with the ability to detect compositional and structural changes within the ECM and integrate this information into a network of signaling circuits that coordinate context dependent cell behavior. Among the most well studied integrins known to play a role in angiogenesis is αvβ3. The complexity by which αvβ3 regulates angiogenesis is illustrated by the fact that this receptor may exhibit both pro and anti-angiogenic functions. It is indicated that the distinct biological responses stimulated by binding to αvβ3 may depend on many factors including the mechanical and biochemical features of the particular ligands, the cell types within which αvβ3 is expressed as well as the concentration and manner by which the ligands are presented to the receptor. For example, studies indicate that αvβ3 binding to specific NC1 domains of collagen or selected RGD peptides can induce apoptosis, induce arteriole contraction and inhibit angiogenesis while other αvβ3 ligands may promote cell survival, induce vascular dilation and support angiogenesis. These observations are consistent with the notion that the final outcome of αvβ3-mediated signaling may depend to a large extent on the particular characteristics of the ligand. While a wealth of data has shown that RGD peptides can inhibit angiogenesis when administered exogenously, the approach of using a cyclic RGD peptide to control tumor growth failed to significantly impact glioblastoma progression and patient survival in late stage clinical testing. Interestingly, studies have indicated that specific RGD peptides may active β3 integrins and under defined experimental conditions induce angiogenesis and tumor growth. These findings and other studies suggesting that amino acids C-terminal to the RGD motif play roles in governing integrin selective binding, prompted the examination of the biological significance of naturally occurring RGD containing epitopes on angiogenesis

For example, multiple pro-angiogenic roles have been proposed for αvβ3 as cyclic arginine-glycineaspartic acid (RGD) containing peptides and antibodies targeting this integrin inhibit angiogenesis in animal models. In contrast, enhanced angiogenesis was detected in tumors growing in αvβ3 null mice. Interestingly, reduced pathological angiogenesis was detected in transgenic mice expressing signaling deficient 3 integrin, which resulted in part from defective recruitment of bone marrow derived cells rather than specific endothelial cell defects. Moreover, evidence suggests that αvβ3 integrin may play a more prominent role in the early stages of angiogenesis when new vessels begin to form, as reduction in endothelial cell expression of αvβ3 integrin impaired early stage pathological angiogenesis, but had little effect on later maturation stages once vessels had formed. These studies, together with many others suggests αvβ3-mediated regulation of angiogenesis is complex, temporally regulated and is not solely dependent on adhesive events, but also involves downstream signaling, the consequences of which may depend on the cell type and composition of the local extracellular microenvironment. Because of the opposing biological responses observed following modulation of some angiogenic regulatory molecules, it is not surprising that anti-angiogenic strategies based on targeting these factors have met with limited clinical success.

Given the importance of integrin-extracellular matrix (ECM) interactions in modulating the intensity and specificity of growth factor signaling, it is important to define how diverse components within the local vascular microenvironment function cooperatively to regulate angiogenesis. Interestingly, distinct αvβ3 ligands may stimulate opposing biological outcomes. For example, certain NC1 domains of collagen may bind αvβ3 and induce proapoptotic responses while binding of other αvβ3 ligands may promote cell growth and survival. Given these findings and the complex biological effects observed following direct targeting of αvβ3, an alternative therapeutic approach to control signaling from αvβ3 might involve specific targeting of the pro-angiogenic ligands of αvβ3 rather than directly targeting the receptor itself. Proteolytic remodeling of the ECM can generate integrin binding cryptic epitopes that play functional roles in angiogenesis including the LPGxPG containing HU177 cryptic epitope present in multiple types of collagen and the HUIV26 cryptic epitope, which is present in collagen type-IV. While the HUIV26 epitope is recognized by αvβ3, it is not specifically composed of an RGD motif.

Sequence analysis of RGD sites within collagen type-I indicate that the KGE tri-peptide motif that is C-terminal to the RGD site was highly conserved among diverse species, while considerable variation is observed in the other collagen RGD flanking sequences. While all five of the collagen RGD epitopes can support cell binding, the highly conserved RGDKGE (SEQ ID NO: 1) collagen peptide P-2 may play a functional role angiogenesis and inflammation given that Mab XL313 directed to this epitope but not an antibody that recognizes the other three RGD collagen sites inhibited angiogenesis and inflammation in vivo. While the precise difference between the three other naturally occurring non-RGDKGE (SEQ ID NO: 1) containing collagen epitopes, one or more of these epitopes were detected in vivo in addition to the RGDKGE (SEQ ID NO: 1) epitope. Given that these RGD containing epitopes are thought to be largely cryptic and not readily accessible to cell surface receptor, the findings are consistent with active collagen remodeling resulting in generation neoepitopes during new vessel formation.

Because of the importance of RGD sequences in mediating some integrin-dependent interactions and the roles of amino acids flanking the core RGD motif in establishing integrin-binding specificity and affinity, the ability of RGD motifs within collagen differentially regulate angiogenesis was determined. Sequence analysis of collagen type-I revealed that five different cryptic RGD motifs are present, each with unique flanking sequences. Surprisingly, the C-terminal KGE flanking sequence of one of these RGD motifs is highly conserved in species as diverse as Xenopus and man. In contrast, significant sequence and positional variation exists within the other flanking sequences among different species.

ECM remodeling occurs as an early event during angiogenesis and multiple proteolytic enzymes including matrix metalloproteinase (MMPs) as well as serine and cysteine proteases the have the capacity to degrade intact or structurally altered forms of collagen. While the in vitro studies indicate that MMP-2-mediated degradation of collagen can lead to the generation of low molecular weights fragments recognized by Mab XL313, the precise mechanism by which the RGDKGE (SEQ ID NO: 1) collagen epitope is generated in vivo is not completely understood. Analysis of angiogenic CAM tissues suggests that a subset of macrophages may be an important source of the RGDKGE (SEQ ID NO: 1) epitope. Activated macrophages with M2-like characteristics can express multiple enzymes capable of degrading collagen and in turn can internalize and further degrade collagen into small low molecular weight fragments. While little evidence exist that macrophages generate and deposit intact triple helical collagen type-I, studies have indicated that certain isoforms of collagen may be expressed. Consistent with previous reports, intact collagen was detected; however low molecular weight RGDKGE (SEQ ID NO: 1) containing collagen fragments in both whole cell lysates and serum free conditioned medium from macrophages like cell lines was detected. While the studies do not rule out macrophage mediated collagen internalization as a contributing factor to the in vivo generation the RGDKGE (SEQ ID NO: 1) collagen epitope, the in vitro studies were carried out in the absence of serum or exogenously added collagen, and thus are consistent with the active generation of the RGDKGE (SEQ ID NO: 1) collagen fragment by macrophages.

Activated macrophages including M2-polarized macrophages have been implicated in supporting angiogenesis and inflammation as multiple factors secreted by these cells exhibit pro-angiogenic activities. While many studies indicate that synthetic RGD containing peptides inhibit angiogenesis and tumor growth, the findings provide the first evidence that macrophages may generate and release an RGDKGE (SEQ ID NO: 1) containing collagen epitope that may exhibit pro-angiogenic activity. Importantly, previous studies have suggested that certain RGD-peptides can activate αvβ3 and may enhance vascular permeability, which might lead to release of inflammatory factors, which may in turn contribute to the formation of new blood vessels.

To examine possible mechanisms by which the RGDKGE (SEQ ID NO: 1) collagen peptide might regulate angiogenesis endothelial cell receptors for this motif were identified. While the possibility that additional non-integrin receptors may bind this collagen epitope is not ruled out, the data suggest that αvβ3 can function as an endothelial cell receptor for the RGDKGE (SEQ ID NO: 1) motif. Interestingly, αvβ3 bound both the RGDKGE (SEQ ID NO: 1) and RGDAPG (SEQ ID NO: 11) collagen peptides, yet only RGDKGE (SEQ ID NO: 1) peptide significantly induced angiogenesis and inflammation in vivo. These findings are consistent with the notion that distinct RGD containing αvβ3 ligands may promote different biological responses. Signaling downstream from αvβ3 is complex and studies have indicated that simple binding of β3 integrin does not necessarily lead to productive outside-in integrin signaling (61). In fact, the capacity of β3 integrins to promote outside-in signaling depends on multiple factors including the extent of receptor clustering and subsequent generation of mechanical tension within the actin cytoskeleton, recruitment of adaptor and accessory proteins such as Gal3, and Kindlin-2 and the association of the integrin with protein tyrosine phosphatases and certain growth factor receptors. While the exact mechanisms leading to RGDKGE (SEQ ID NO: 1)-mediated αvβ3 signaling is not completely understood, endothelial cell interactions with the RGDKGE (SEQ ID NO: 1) peptide in the absence of serum led to enhanced phosphorylation of β3 integrin on tyrosine 747 and of Src phosphorylation at tyrosine 416. These data and others are consistent with an early mechanical mediated activation of β3 integrin that depends on Src given that blocking Src activity reduced B3 phosphorylation following binding to the RGDKGE (SEQ ID NO: 1) motif.

Integrin signaling and Src activation are known to regulate the architecture of the actin cytoskeleton. Moreover, Src family kinases regulate P38MAPK, and activation of P38MAPK is thought to enhance actin stress fiber formation in endothelial cells and regulate angiogenesis in vivo. The findings provide insight into the coordinated roles of P38MAPK and Src in regulating RGD-dependent endothelial cell signaling through αvβ3 as interactions with the RGDKGE (SEQ ID NO: 1) cryptic collagen epitope leads to enhanced P38MAPK phosphorylation in a Src-dependent manner. Moreover, RGDKGE (SEQ ID NO: 1)-induced angiogenesis in vivo was associated with enhanced levels of phosphorylated P38MAPK, and this angiogenic response was reduced by an inhibitor of P38MAPK. These findings are consistent with the notion that RGDKGE (SEQ ID NO: 1) stimulated angiogenesis depends on P38MAPK.

Recent studies have suggested a role for actin stress fibers and mechanical tension in promoting nuclear accumulation of YAP (Yes-Associated Protein), where it is thought to function in conjunction with TEA Domain (TEAD) transcription factors (e.g., TEAD1 (TEF-1/NTEF), TEAD2 (TEF-4/ETF), TEAD3 (TEF-5/ETFR-1), and TEAD4 (TEF-3/ETFR-2/FR-19)) in regulating gene expression. Given data suggesting a role for YAP in regulating endothelial cell growth and angiogenesis, the subcellular distribution of YAP in endothelial cells following interaction with the RGDKGE (SEQ ID NO: 1) collagen peptide was examined. The data indicate that endothelial cell interaction with the RGDKGE (SEQ ID NO: 1) epitope was associated with enhanced nuclear accumulation of YAP. Integrin signaling may play a role in the regulation of YAP as studies have implicated a role for β1 integrins expressed in skeletal stem cells and av integrins expressed in osteoblasts in governing YAP subcellular localization. The findings are consistent with a mechanism by which αvβ3-mediated binding to the RGDKGE (SEQ ID NO: 1) epitope, but not the related RGDAPG (SEQ ID NO: 11) epitope stimulates a signaling cascade leading to enhanced nuclear accumulation of YAP that depends on Src and/or P38MAPK. This possibility is supported by the findings that reduced levels of nuclear YAP was detected following αvβ3-mediated interaction with RGDKGE (SEQ ID NO: 1) peptide in endothelial cells in which Src or P38MAPK was inhibited. Given the documented role of YAP in governing cell growth coupled with the ability of the RGDKGE (SEQ ID NO: 1) collagen peptide to stimulate nuclear accumulation of YAP and enhance endothelial cell growth, it is possible that the RGDKGE (SEQ ID NO: 1) collagen peptide-induced endothelial cell growth is YAP dependent. Consistent with this possibility, no enhancement of endothelial cell growth was detected following knockdown of YAP in endothelial cells stimulated with the RGDKGE (SEQ ID NO: 1) collagen peptide, even though these cells are capable of proliferating as stimulation with VEGF or high levels of serum enhanced their growth. Given the studies, it is possible that part of the FGF-2 induced angiogenic response observed in the chick CAM model might involve the recruitment of macrophages that generate a previously uncharacterized RGDKGE (SEQ ID NO: 1) containing cryptic collagen epitope that binds to αvβ3 leading to Src and P38MAPK activation and nuclear accumulation of YAP. Given that YAP is known to regulate a wide array of genes that may impact angiogenesis and inflammation including CTGF and Cry61, it is likely that the RGDKGE (SEQ ID NO: 1) collagen epitope may initiate a complex pro-angiogenic program in vivo involving YAP-associated regulation of multiple pro-angiogenic molecules and not simply be restricted to only enhancing endothelial cell growth.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

Unless specifically stated or obvious from context, as used herein, the terms “a”, “an”, and “the” are understood to be singular or plural.

Unless specifically stated or obvious from context, as used herein, the term “or” is understood to be inclusive.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

The terms “reduce”, “decrease” or “inhibit” refer to altering negatively by at least 5%. The terms “increase”, “induce” or “enhance” refer to altering positively by at least 5%. The term “modulate” refers to altering negatively or positively by at least 5%. An alteration may be by 5%, 10%, 25%, 30%, 50%, 75%, or even by 100%.

By “agent” is meant any small molecule chemical compound, antibody, nucleic acid molecule, or polypeptide, or fragments thereof.

By “blood vessel formation” is meant the dynamic process that includes one or more steps of blood vessel development and/or maturation, such as angiogenesis, arteriogenesis, vasculogenesis, formation of an immature blood vessel network, blood vessel remodeling, blood vessel stabilization, blood vessel maturation, blood vessel differentiation, or establishment of a functional blood vessel network.

By “blood vessel remodeling” or “vascular remodeling” is meant the dynamic process of blood vessel enlargement in shape and size to maintain the luminal orifice and blood flow. For example, vascular remodeling includes change in arterial size to adapt to plaque accumulation, effectively maintaining the lumen and blood flow to the myocardium.

By “control” or “reference” is meant a standard of comparison. As used herein, “changed as compared to a control” sample or subject is understood as having a level of the analyte or diagnostic or therapeutic indicator to be detected at a level that is statistically different than a sample from a normal, untreated, or control sample. Control samples include, for example, cells in culture, one or more laboratory test animals, or one or more human subjects. Methods to select and test control samples are within the ability of those in the art. An analyte can be a naturally occurring substance that is characteristically expressed or produced by the cell or organism (e.g., an antibody, a protein) or a substance produced by a reporter construct (e.g, B-galactosidase or luciferase). Depending on the method used for detection the amount and measurement of the change can vary. Determination of statistical significance is within the ability of those skilled in the art, e.g., the number of standard deviations from the mean that constitute a positive result. In some embodiments, the control or reference comprises the level of tumor growth and/or size prior to administration of the ADC wherein the antibody, or the antigen binding fragment thereof binds to collagen. In some embodiments, the control or reference comprises a standard derived from efficacy measurements of an ADC composition administered to subjects without an antagonist of collagen or a functional fragment thereof. In some embodiments, the control comprises an antibody that does not recognize or bind to a cryptic collagen epitope. In some embodiments the control comprises an antibody that does not recognize or bind to a cryptic collagen epitope, but is conjugated to a drug. In some embodiments, the control comprises an antibody that binds to a cryptic collagen epitope, but is not conjugated to a drug. In some embodiments, the control or reference is a measurement of cytotoxicity in a tumor or a cancel cell in the subject prior to the administration of the ADC, wherein the antibody, or the antigen binding fragment thereof binds to collagen.

As used herein, the term “cytotoxicity” refers to the quality of being toxic to cells. A cytotoxic agent may cause the induction or activation of cellular pathways or cascades that inhibit or prevent cell function and/or cause cell destruction or result in cell death. Cells exposed to cytotoxic compounds may undergo necrosis (uncontrolled cell death), apoptosis (programmed cell death), autophagy, lysis, or stop actively growing and dividing to decrease cell proliferation. In some embodiments, the ADCs of the disclosure comprising an antibody, or an antigen binding portion thereof conjugated to at least one drug, wherein the antibody, or the antigen binding fragment thereof binds to collagen, are cytotoxic, i.e. inhibit or prevent cell function and/or cause cell destruction.

As used herein, “cell death” refers to the event of a cell ceasing to carry out its functions. Cell death could occur as a result of multiple processes, including apoptosis (programmed cell death that occurs during growth and development, and also in response to harmful environmental stimuli), autophagy (cellular degradation and recycling) or necrosis (accidental or programmed cell death that may result in tissue damage). In some embodiments, the ADCs of the disclosure comprising an antibody, or an antigen binding portion thereof conjugated to at least one drug, wherein the antibody, or the antigen binding fragment thereof binds to collagen, induce cell death of cancer cells or of tumor cells. In some embodiments, the cell death induced by the ADCs of the invention is at least 1.1 fold, at least 1.5 fold, at least 2 fold, at least 5 fold, at least 10 fold, at least 20 fold, at least 50 fold, at least 100 fold, at least 200 fold higher compared to a control.

As used herein, the term “lysis” refers to the breakdown of the membrane of a cell caused by damage to its plasma (outer) membrane. An impairment in the condition of the cell membrane (“membrane integrity”) may be caused by chemical or physical means, or through the action of a biological agent such as a virus. In some embodiments, the ADCs of the disclosure comprising an antibody, or an antigen binding portion thereof conjugated to at least one drug, wherein the antibody, or the antigen binding fragment thereof binds to collagen, cause the lysis of cancer cells or tumor cells.

As used herein, “detecting”, “detection” and the like are understood that an assay performed for identification of a specific analyte in a sample, e.g., an antigen in a sample or the level of an antigen in a sample. The amount of analyte or activity detected in the sample can be none or below the level of detection of the assay or method.

The term “polynucleotide” or “nucleic acid” as used herein designates mRNA, RNA, CRNA, cDNA or DNA. As used herein, a “nucleic acid encoding a polypeptide” is understood as any possible nucleic acid that upon (transcription and) translation would result in a polypeptide of the desired sequence. The degeneracy of the nucleic acid code is well understood. Further, it is well known that various organisms have preferred codon usage, etc. Determination of a nucleic acid sequence to encode any polypeptide is well within the ability of those of skill in the art.

As used herein, “immunoassay” is understood as any antibody base detection method including, but not limited to enzyme linked immunosorbent assay (ELISA), radioimmune assay (RIA), Western blot, immunohistochemistry, immunoprecipitation assay such as Luciferase Immunoprecipitation System (LIPS see, e.g., US Patent Publication 2007/0259336 which is incorporated herein by reference). In a preferred embodiment, the immunoassay is a quantitative. Antibodies for use in immunoassays include any monoclonal or polyclonal antibody appropriate for use in the specific immunoassay.

By “inhibitory nucleic acid molecule” is meant a polynucleotide that disrupts the expression of a target nucleic acid molecule or an encoded polypeptide. Exemplary inhibitory nucleic acid molecules include, but are not limited to, shRNAs, siRNAs, antisense nucleic acid molecules, and analogs thereof.

As used herein, the term “internalization” is to be understood as involving the localization of at least a part of a molecule that passes through the plasma membrane into the cytoplasm, vesicles, endosomes, organelles or nucleus of a cell. Any possible mechanism of internalization is envisaged including both energy-dependent (i.e. active) transport mechanisms (e.g., endocytosis) and energy-independent (i.e. passive) transport mechanism (e.g., diffusion). The term “internalized in a cell”, as used herein, refers to the ability of ADCs of the disclosure comprising an antibody, or an antigen binding portion thereof conjugated to at least one drug, wherein the antibody, or the antigen binding fragment thereof binds to collagen, to pass cellular membranes, including, but not limited to, the outer cell membrane, plasma membrane, endosomal membranes, and membranes of the endoplasmic reticulum, or to direct the passage of a given agent or cargo through these cellular membranes.

As used herein, “isolated” or “purified” when used in reference to a polypeptide means that a naturally polypeptide or protein has been removed from its normal physiological environment (e.g., protein isolated from plasma or tissue, optionally bound to another protein) or is synthesized in a non-natural environment (e.g., artificially synthesized in an in vitro translation system or using chemical synthesis). Thus, an “isolated” or “purified” polypeptide can be in a cell-free solution or placed in a different cellular environment (e.g., expressed in a heterologous cell type). The term “purified” does not imply that the polypeptide is the only polypeptide present, but that it is essentially free (about 90-95%, up to 99-100% pure) of cellular or organismal material naturally associated with it, and thus is distinguished from naturally occurring polypeptide. Similarly, an isolated nucleic acid is removed from its normal physiological environment. “Isolated” when used in reference to a cell means the cell is in culture (i.e., not in an animal), either cell culture or organ culture, of a primary cell or cell line. Cells can be isolated from a normal animal, a transgenic animal, an animal having spontaneously occurring genetic changes, and/or an animal having a genetic and/or induced disease or condition. An isolated virus or viral vector is a virus that is removed from the cells, typically in culture, in which the virus was produced.

As used herein, “plurality” is understood to mean more than one. For example, a plurality refers to at least two, three, four, five, or more.

A “polypeptide” or “peptide” as used herein is understood as two or more independently selected natural or non-natural amino acids joined by a covalent bond (e.g., a peptide bond). A peptide can include 2, 3, 4, 5, 6, 7, 8, 9, 10, 11, 12, 13, 14, 15, 16, 17, 18, 19, 20, or more natural or non-natural amino acids joined by peptide bonds. Polypeptides as described herein include full length proteins (e.g., fully processed proteins) as well as shorter amino acids sequences (e.g., fragments of naturally occurring proteins or synthetic polypeptide fragments). Optionally the peptide further includes one or more modifications such as modified peptide bonds, i.e., peptide isosteres, and may contain amino acids other than the 20 gene-encoded amino acids. The polypeptides may be modified by either natural processes, such as posttranslational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formulation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, Proteins, Structure and Molecular Properties, 2nd ed., T. E. Creighton, W.H. Freeman and Company, New York (1993); Posttranslational Covalent Modification of Proteins, B. C. Johnson, ed., Academic Press, New York, pgs. 1-12 (1983); Seifter et al., Meth. Enzymol 182:626-646 (1990); Rattan et al., Ann. N.Y. Acad. Sci. 663:48-62 (1992)).

“Sensitivity and specificity” are statistical measures of the performance of a binary classification test. The sensitivity (also called recall rate in some fields) measures the proportion of actual positives which are correctly identified as such (e.g. the percentage of sick people who are identified as having the condition); and the specificity measures the proportion of negatives which are correctly identified (e.g. the percentage of well people who are identified as not having the condition). They are closely related to the concepts of type I and type II errors. A theoretical, optimal prediction can achieve 100% sensitivity (i.e. predict all people from the sick group as sick) and 100% specificity (i.e. not predict anyone from the healthy group as sick).

The concepts are expressed mathematically as follows:
sensitivity= #true positives/#true positives+ #false negatives
specificity= #true negatives/#true negatives+ #false positives.

By “selectively” is meant the ability to affect the activity or expression of a target molecule without affecting the activity or expression of a non-target molecule.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

As used herein “tumor-infiltrating lymphocytes” or TILs refer to white blood cells that have left the bloodstream and migrated into a tumor. Lymphocytes can be divided into three groups including B cells, T cells and natural killer cells. As used herein “T-cells” refers to CD3+ cells, including CD4+ helper cells, CD8+ cytotoxic T-cells and y& T cells.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### Antibody Drug Conjugate

### Antibody

The term “antibody-drug-conjugate” or “ADC” refers to a binding protein, such as an antibody or antigen binding fragment thereof, chemically linked to one or more chemical drug(s) (also referred to herein as agent(s)) that may optionally be therapeutic or cytotoxic agents. In a preferred embodiment, an ADC includes an antibody, a cytotoxic or therapeutic drug, and a linker that enables attachment or conjugation of the drug to the antibody. An ADC typically has anywhere from 1 to 8 drugs conjugated to the antibody, including drug loaded species of 2, 4, 6, or 8. Non-limiting examples of drugs that may be included in the ADCs are mitotic inhibitors, antitumor antibiotics, immunomodulating agents, vectors for gene therapy, alkylating agents, antiangiogenic agents, antimetabolites, boron-containing agents, chemoprotective agents, hormones, antihormone agents, corticosteroids, photoactive therapeutic agents, oligonucleotides, radionuclide agents, topoisomerase inhibitors, tyrosine kinase inhibitors, and radiosensitizers.

The term “extracellular-targeted drug conjugate” or “EDC” refers to a drug conjugate of the invention in which an antibody or other targeting moiety that targets an extracellular target is linked via a stable or non-cleavable linker to a drug that binds to an extracellular target.

The term “extracellular” refers to proteins, antigens, or epitopes located on the external portion of a cell membrane or are in the extracellular matrix (for example, collagen).

By “antibody” is meant any immunoglobulin polypeptide, or fragment thereof, having immunogen binding ability. As used herein, the term “antibodies” includes polyclonal antibodies, affinity-purified polyclonal antibodies, monoclonal antibodies, and antigen-binding fragments, such as F(ab′) 2 and Fab proteolytic fragments. Genetically engineered intact antibodies or fragments, such as chimeric antibodies, Fv fragments, single chain antibodies, and the like, as well as synthetic antigen-binding peptides and polypeptides, are also included. Non-human antibodies may be humanized by grafting non-human CDRs onto human framework and constant regions, or by incorporating the entire non-human variable domains. In certain preferred embodiments, humanized antibodies may retain non-human residues within the human variable region framework domains to enhance proper binding characteristics.

The term “humanized antibody” refers to antibodies which comprise heavy and light chain variable region sequences from a nonhuman species (e.g., a mouse) but in which at least a portion of the VH and/or VL sequence has been altered to be more “human-like”, i.e., more similar to human germline variable sequences. In particular, the term “humanized antibody” is an antibody or a variant, derivative, analog or fragment thereof which immunospecifically binds to an antigen of interest and which comprises a framework (FR) region having substantially the amino acid sequence of a human antibody and a complementary determining region (CDR) having substantially the amino acid sequence of a non-human antibody. As used herein, the term “substantially” in the context of a CDR refers to a CDR having an amino acid sequence at least 80%, preferably at least 85%, at least 90%, at least 95%, at least 98% or at least 99% identical to the amino acid sequence of a non-human antibody CDR. A humanized antibody comprises substantially all of at least one, and typically two, variable domains (Fab, Fab′, F(ab′) 2. FabC, Fv) in which all or substantially all of the CDR regions correspond to those of a non-human immunoglobulin (i.e., donor antibody) and all or substantially all of the framework regions are those of a human immunoglobulin consensus sequence. Preferably, a humanized antibody also comprises at least a portion of an immunoglobulin constant region (Fc), typically that of a human immunoglobulin. In some embodiments, a humanized antibody contains both the light chain as well as at least the variable domain of a heavy chain. The antibody also may include the CH1, hinge, CH2, CH3, and CH4 regions of the heavy chain. In some embodiments, a humanized antibody only contains a humanized light chain. In other embodiments, a humanized antibody only contains a humanized heavy chain. In specific embodiments, a humanized antibody only contains a humanized variable domain of a light chain and/or humanized heavy chain.

The humanized antibody can be selected from any class of immunoglobulins, including IgM, IgG. IgD. IgA and IgE, and any isotype, including without limitation IgG1. IgG2, IgG3 and IgG4. The humanized antibody may comprise sequences from more than one class or isotype, and particular constant domains may be selected to optimize desired effector functions using techniques well-known in the art.

Exemplary Sequences of Antibodies provided in Table 1.

TABLE 1

| Description | Sequence |
|---|---|
| Humanized anti- | MGWTLVFLFLLSVTAGVHSQVTLKESGPALVKPTQTLTLTCTFS |
| HU177 heavy chain | GFSLSTPGMGVWWIRQPPGKALEWLADIWWDDNKYTNPSLKS |
| (SEQ ID NO: 8) | RLTISKDTSKNQVVLTMTNMDPVDTATYYCARRANYGNPYYA |
| | QDYWGQGTTVTVSSASTKGPSVFPLAPSSKSTSGGTAALGCLV |
| | KDYFPEPVTVSWNSGALTSGVHTFPAVLQSSGLYSLSSVVTVPS |
| | SSLGTQTYICNVNHKPSNTKVDKKVEPKSCDKTHTCPPCPAPEL |
| | LGGPSVFLFPPKPKDTLMISRTPEVTCVVVDVSHEDPEVKENWY |
| | VDGVEVHNAKTKPREEQYNSTYRVVSVLTVLHQDWLNGKEY |
| | KCKVSNKALPAPIEKTISKAKGQPREPQVYTLPPSRDELTKNQV |
| | SLTCLVKGFYPSDIAVEWESNGQPENNYKTTPPVLDSDGSFFLY |
| | SKLTVDKSRWQQGNVFSCSVMHEALHNHYTQKSLSLSPGK |
| Humanized anti- | MVSSAQFLGLLLLCFQGTRCDIVMTQTPLSLPVTPGEPASISCRS |
| HU177 light chain | SQSIVSSWGNTYLEWYLOKPGQSPQLLIYKVSNRFSGVPDRESG |
| (SEQ ID NO: 9) | SGSGTDFTLKISRVEAEDVGVYYCFQGSHVPWTFGQGTKVEIK |
| | RTVAAPSVFIFPPSDEQLKSGTASVVCLLNNFYPREAKVQWKV |
| | DNALQSGNSQESVTEQDSKDSTYSLSSTLTLSKADYEKHKVYA |
| | CEVTHQGLSSPVTKSFNRGEC |

“Antigenic fragment” and the like are understood as at least that portion of a peptide capable of inducing an immune response in a subject, or being able to be specifically bound by an antibody raised against the antigenic fragment. Typically, antigenic fragments are at least 7 amino acids in length. Antigenic fragments can include deletions of the amino acid sequence from the N-terminus or the C-terminus, or both. For example, an antigenic fragment can have an N- and/or a C-terminal deletion of about 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, or more amino acids. Antigenic fragments can also include one or more internal deletions of the same exemplary lengths. Antigenic fragments can also include one or more point mutations, particularly conservative point mutations. At least an antigenic fragment of protein can include the full length, wild-type sequence of the antigen. An antigenic fragment can include more than one potential antibody binding site. An antigenic fragment can be used to make antibodies for use in any of the methods provided herein.

The term “antigen” refers to the substance or target that an antibody or targeting moiety binds. An antigen is characterized by its ability to be “bound” by the antibody or targeting moiety. Antigen can also mean the substance used to elicit the production of targeting moieties, such as the production of antigen specific antibodies through immunizing with the antigen.

As used herein, “binding” or “specific binding” is understood as having at least a 103 or more, preferably 104 or more, preferably 105 or more, preferably 106 or more preference for binding to a specific binding partner as compared to a non-specific binding partner (e.g., binding an antigen to a sample known to contain the cognate antibody).

The term “antigen binding site” or “epitope” refers to the portion of the antigen to which a targeting moiety, such as an antibody, binds.

### Exemplary Linkers

The term “linker” refers to a chemical moiety or bond that covalently attaches two or more molecules, such as a targeting moiety and a drug.

The term “linker spacer group” refers to atoms in the linker that provide space between the two molecules joined by the linker.

For example, the linker may include a spacer, which is a moiety that extends the drug linkage to avoid, for example, shielding the active site of the antibody or improving the solubility of the ADC. Other examples of components of linkers include a stretcher unit and an amino acid unit.

Two suitable methods for conjugating drugs to antibodies include: alkylation of reduced interchain cysteine disulfides through an enzymatically non-cleavable maleimido or a simple and cleavable disulfide linker, or acylation of lysines by cleavable linear amino acids.

In one aspect, a linker covalently attaches an antibody to a drug moiety. An ADC is prepared using a linker having reactive functionality for binding to the antibody and the drug. For example, a cysteine thiol, or an amine, e.g., N-terminus or amino acid side chain such as lysine, of the antibody may form a bond with a functional group of the linker.

Suitable linkers include, for example, cleavable and non-cleavable linkers. A linker may be a “cleavable linker,” facilitating release of a drug. Nonlimiting exemplary cleavable linkers include acid-labile linkers (e.g., comprising hydrazone), protease-sensitive (e.g., peptidase-sensitive) linkers, photolabile linkers, or disulfide-containing linkers (Chari et al., Cancer Research 52:127-131 (1992); U.S. Pat. No. 5,208,020). A cleavable linker is typically susceptible to cleavage under intracellular conditions. Suitable cleavable linkers include, for example, a peptide linker cleavable by an intracellular protease, such as lysosomal protease or an endosomal protease. In exemplary embodiments, the linker can be a dipeptide linker, such as a valine-citrulline (val-cit) or a phenylalanine-lysine (phe-lys) linker.

Linkers are preferably stable extracellularly in a sufficient manner to be therapeutically effective. Before transport or delivery into a cell, the ADC is preferably stable and remains intact, i.e. the antibody remains conjugated to the drug moiety. Linkers that are stable outside the target cell may be cleaved at some efficacious rate once inside the cell. Thus, an effective linker will: (i) maintain the specific binding properties of the antibody; (ii) allow delivery, e.g., intracellular delivery, of the drug moiety; and (iii) maintain the therapeutic effect, e.g., cytotoxic effect, of a drug moiety.

In one embodiment, the linker is cleavable under intracellular conditions, such that cleavage of the linker sufficiently releases the drug from the antibody in the intracellular environment to be therapeutically effective. In some embodiments, the cleavable linker is pH-sensitive, i.e., sensitive to hydrolysis at certain pH values. Typically, the pH-sensitive linker is hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used. (Sec, e.g., U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661.) Such linkers are relatively stable under neutral pH conditions, such as those in the blood, but are unstable at below pH 5.5 or 5.0, the approximate pH of the lysosome. In certain embodiments, the hydrolyzable linker is a thioether linker (such as, e.g., a thioether attached to the therapeutic agent via an acylhydrazone bond (see, e.g., U.S. Pat. No. 5,622,929).

In other embodiments, the linker is cleavable under reducing conditions (e.g., a disulfide linker). A variety of disulfide linkers are known in the art, including, for example, those that can be formed using SATA (N-succinimidyl-5-acetylthioacetate), SPDP (N-succinimidyl-3-(2-pyridyldithio) propionate), SPDB (N-succinimidyl-3-(2-pyridyldithio) butyrate) and SMPT (N-succinimidyloxycarbonyl-alpha-methyl-alpha-(2-pyridyl-dithio) toluene), SPDB and SMPT. (See, e.g., Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935.).

In some embodiments, the linker is cleavable by a cleaving agent, e.g., an enzyme, that is present in the intracellular environment (e.g., within a lysosome or endosome or caveolea). The linker can be, e.g., a peptidyl linker that is cleaved by an intracellular peptidase or protease enzyme, including, but not limited to, a lysosomal or endosomal protease. In some embodiments, the peptidyl linker is at least two amino acids long or at least three amino acids long. Cleaving agents can include cathepsins B and D and plasmin, all of which are known to hydrolyze dipeptide drug derivatives resulting in the release of active drug inside target cells (see, e.g., Dubowchik and Walker, 1999, Pharm. Therapeutics 83:67-123). Most typical are peptidyl linkers that are cleavable by enzymes that are present in ILT3-expressing cells. Examples of such linkers are described, e.g., in U.S. Pat. No. 6,214,345, incorporated herein by reference in its entirety and for all purposes. In a specific embodiment, the peptidyl linker cleavable by an intracellular protease is a Val-Cit linker or a Phe-Lys linker (see, e.g., U.S. Pat. No. 6,214,345, which describes the synthesis of doxorubicin with the val-cit linker). One advantage of using intracellular proteolytic release of the therapeutic agent is that the agent is typically attenuated when conjugated and the serum stabilities of the conjugates are typically high.

In other embodiments, the linker is a malonate linker (Johnson et al., 1995, Anticancer Res. 15:1387-93), a maleimidobenzoyl linker (Lau et al., 1995, Bioorg-Med-Chem. 3 (10): 1299-1304), or a 3′-N-amide analog (Lau et al., 1995, Bioorg-Med-Chem. 3 (10): 1305-12).

In yet other embodiments, the linker unit is not cleavable and the drug is released, for example, by antibody degradation. See U.S. Publication No. 20050238649 incorporated by reference herein in its entirety. An ADC comprising a non-cleavable linker may be designed such that the ADC remains substantially outside the cell and interacts with certain receptors on a target cell surface such that the binding of the ADC initiates (or prevents) a particular cellular signaling pathway.

In some embodiments, the linker is substantially hydrophilic linker (e.g., PEG4Mal and sulfo-SPDB). A hydrophilic linker may be used to reduce the extent to which the drug may be pumped out of resistant cancer cells through MDR (multiple drug resistance) or functionally similar transporters.

In other embodiments, upon cleavage, the linker functions to directly or indirectly inhibit cell growth and/or cell proliferation. For example, in some embodiments, the linker, upon cleavage, can function as an intercalating agent, thereby inhibiting macromolecular biosynthesis (e.g. DNA replication, RNA transcription, and/or protein synthesis).

In some embodiments, a linker component comprises an “amino acid unit.” In some such embodiments, the amino acid unit allows for cleavage of the linker by a protease, thereby facilitating release of the drug from the immunoconjugate upon exposure to intracellular proteases, such as lysosomal enzymes (Doronina et al. (2003) Nat. Biotechnol. 21:778-784). Exemplary amino acid units include, but are not limited to, dipeptides, tripeptides, tetrapeptides, and pentapeptides. Exemplary dipeptides include, but are not limited to, valine-citrulline (vc or val-cit), alanine-phenylalanine (af or ala-phe); phenylalanine-lysine (fk or phe-lys); phenylalanine-homolysine (phe-homolys); and N-methyl-valine-citrulline (Me-val-cit). Exemplary tripeptides include, but are not limited to, glycine-valine-citrulline (gly-val-cit) and glycine-glycine-glycine (gly-gly-gly). An amino acid unit may comprise amino acid residues that occur naturally and/or minor amino acids and/or non-naturally occurring amino acid analogs, such as citrulline Amino acid units can be designed and optimized for enzymatic cleavage by a particular enzyme, for example, a tumor-associated protease, cathepsin B, C and D, or a plasmin protease.

In some embodiments, the linker is Cathepsin B cleavable VC-PAB linkage.

The conjugation of the drug to the antibody via a linker can be accomplished by any technique known in the art. A number of different reactions are available for covalent attachment of drugs and linkers to antibodies. This may be accomplished by reaction of the amino acid residues of the antibody, including the amine groups of lysine, the free carboxylic acid groups of glutamic and aspartic acid, the sulfhydryl groups of cysteine and the various moieties of the aromatic amino acids. One of the most commonly used non-specific methods of covalent attachment is the carbodiimide reaction to link a carboxy (or amino) group of a compound to amino (or carboxy) groups of the antibody. Additionally, bifunctional agents such as dialdehydes or imidoesters have been used to link the amino group of a compound to amino groups of an antibody. Also available for attachment of drugs to antibodies is the Schiff base reaction. This method involves the periodate oxidation of a drug that contains glycol or hydroxy groups, thus forming an aldehyde which is then reacted with the binding agent. Attachment occurs via formation of a Schiff base with amino groups of the antibody. Isothiocyanates can also be used as coupling agents for covalently attaching drugs to antibodies. Other techniques are known to the skilled artisan and within the scope of the disclosure.

The term “drug-to-antibody ratio” or “DAR” refers to the number of drugs, e.g., IGN, auristatin, or maytansinoid, attached to the antibody of the ADC. The DAR of an ADC can range from 1 to 8, although higher loads, e.g., 10, are also possible depending on the number of linkage site on an antibody. The term DAR may be used in reference to the number of drugs loaded onto an individual antibody, or, alternatively, may be used in reference to the average or mean DAR of a group of ADCs.

### Exemplary Drug

The terms “drug,” “agent,” and “drug moiety” are used interchangeably herein. The terms “linked” and “conjugated” are also used interchangeably herein and indicate that the antibody and moiety are covalently linked.

In some embodiments, the drug is selected from the group consisting of an anti-apoptotic agent, a mitotic inhibitor, an anti-tumor antibiotic, an immunomodulating agent, a nucleic acid for gene therapy, an anti-angiogenic agent, an anti-metabolite, a boron-containing agent, a chemoprotective agent, a hormone agent, an anti-hormone agent, a corticosteroid, a photoactive therapeutic agent, an oligonucleotide, a radionuclide agent, a radiosensitizer, a topoisomerase inhibitor, and a tyrosine kinase inhibitor.

In some embodiments, the mitotic inhibitor is selected from the group consisting of monomethyl auristatin E (MMAE), monomethyl auristatin F(MMAF), mertansine (DM1), and ravtansine (DM4).

In some embodiments, the DNA damaging agent is selected from the group consisting of psilocybin, gliclazomycin, streptomycin, pyrrolobenzodiazepine (PBD), doxorubicin, and adrianmycin.

In some embodiments, the immunomodulating agent comprises an immune checkpoint inhibitor. Suitable immune checkpoint inhibitors comprise an inhibitor of CTLA-4, PD-1, PDL-1, Lag3, LAIR1, or LAIR 2. For example, the immune checkpoint inhibitor comprises a CTLA-4 antibody, a PD-1 antibody, a PDL-1 antibody, a Lag3 antibody, a LAIR1 antibody, or a LAIR 2 antibody.

### Collagen

Suitable types of collagen include collagen type-I, collagen type-II, collagen type-III and collagen type-IV (e.g., the alpha 6 chain of collagen type-IV). In some cases, the antagonist of collagen or a fragment thereof comprises an antagonist of the XL313 cryptic collagen epitope or an antagonist of the HU177 cryptic collagen epitope. For example, the antagonist of the XL313 cryptic collagen epitope comprises an antibody that binds a cryptic RGDKGE (SEQ ID NO: 1) containing collagen epitope or wherein the antagonist of the HU177 cryptic collagen epitope comprises an antibody that binds a cryptic CPGFPGFC (SEQ ID NO: 16)-containing collagen epitope. Preferably, the antibody comprises a monoclonal antibody, e.g., an XL313 monoclonal antibody or an HU177 monoclonal antibody.

Preferably, the antagonist of collagen or a fragment thereof enhances anti-tumor activity of the immune checkpoint inhibitor and inhibits an inflammatory condition. Exemplary inflammatory conditions include dermatitis, pneumonitis, or colitis.

By “cryptic” is meant that a motif may be inaccessible to cell surface receptors, and once the target protein is proteolyzed or denatured, a sequence becomes exposed or generates a fragment that is then recognized by the antibody. For example, the XL313 epitope, i.e., RGDKGE (SEQ ID NO: 1) core sequence within collagen type-I is cryptic in that the antibody does not react with normal collagen in its triple helical state, but once it is proteolyzed or denatured, the sequence becomes exposed or generates a fragment of collagen that is recognized by Mab XL313. Similarly, the HU177 epitope, i.e., CPGFPGFC (SEQ ID NO: 16) core sequence within collagen type-I is cryptic in that the antibody does not react with normal collagen in its triple helical state, but once it is proteolyzed or denatured, the sequence becomes exposed or generates a fragment of collagen that is recognized by Mab HU177. Given that both anti-HU177 and anti-XL313 antibodies recognize and bind to cryptic sequence motifs in collagen-I, it is expected that antibody-drug conjugates of both antibodies would show similar therapeutic activity.

Exemplary collagen sequences are shown in Table 2.

TABLE 2

| Description | Sequence |
|---|---|
| Collagen α1(I) | mfsfvdlrlllllaatallthgqeegqvegqdedippitcvqnglryhdrdvwkpepcricvcdngk |
| (SEQ ID NO: 10) | vlcddvicdetkncpgaevpegeccpvcpdgsesptdqettgvegpkgdtgprgprgpagppgr |
| | dgipgqpglpgppgppgppgppglggnfapqlsygydekstggisvpgpmgpsgprglpgpp |
| | gapgpqgfqgppgepgepgasgpmgprgppgppgkngddgeagkpgrpgergppgpqgar |
| | glpgtaglpgmkghrgfsgldgakgdagpagpkgepgspgengapgqmgprglpgergrpga |
| | pgpagargndgatgaagppgptgpagppgfpgavgakgeagpqgprgsegpqgvrgepgpp |
| | gpagaagpagnpgadgqpgakgangapgiagapgfpgargpsgpqgpggppgpkgnsgepg |
| | apgskgdtgakgepgpvgvqgppgpageegkrgargepgptglpgppgerggpgsrgfpgad |
| | gvagpkgpagergspgpagpkgspgeagrpgeaglpgakgltgspgspgpdgktgppgpagq |
| | dgrpgppgppgargqagvmgfpgpkgaagepgkagergvpgppgavgpagkdgeagaqgp |
| | pgpagpagergeqgpagspgfqglpgpagppgeagkpgeqgvpgdlgapgpsgargergfpg |
| | ergvqgppgpagprgangapgndgakgdagapgapgsqgapglqgmpgergaaglpgpkgd |
| | rgdagpkgadgspgkdgvrgltgpigppgpagapgdkgesgpsgpagptgargapgdrgepg |
| | ppgpagfagppgadgqpgakgepgdagakgdagppgpagpagppgpignvgapgakgarg |
| | sagppgatgfpgaagrvgppgpsgnagppgppgpagkeggkgprgetgpagrpgevgppgp |
| | pgpagekgspgadgpagapgtpgpqgiagqrgvvglpgqrgergfpglpgpsgepgkqgpsg |
| | asgergppgpmgppglagppgesgregapgaegspgrdgspgakgdrgetgpagppgapgap |
| | gapgpvgpagksgdrgetgpagpagpvgpvgargpagpqgprgdkgetgeqgdrgikghrgf |
| | sglqgppgppgspgeqgpsgasgpagprgppgsagapgkdglnglpgpigppgprgrtgdagp |
| | vgppgppgppgppgppsagfdfsflpqppqekahdggryyraddanvvrdrdlevdttlkslsq |
| | qienirspegsrknpartcrdlkmchsdwksgeywidpnqgenldaikvfcnmetgetcvyptq |
| | psvaqknwyisknpkdkrhvwfgesmtdgfqfeyggqgsdpadvaiqltflrlmsteasqnity |
| | hcknsvaymdqqtgnlkkalllqgsneieiraegnsrftysvtvdgctshtgawgktvieykttktsr |
| | lpiidvapldvgapdqefgfdvgpvcfl |
| Collagen α2(I) | mlsfvdtrtllllavtlclatcqslqeetvrkgpagdrgprgergppgppgrdgedgptgppgppgp |
| (SEQ ID NO: 23) | pgppglggnfaaqydgkgvglgpgpmglmgprgppgaagapgpqgfqgpagepgepgqtg |
| | pagargpagppgkagedghpgkpgrpgergvvgpqgargfpgtpglpgfkgirghngldglkg |
| | qpgapgvkgepgapgengtpgqtgarglpgergrvgapgpagargsdgsvgpvgpagpigsag |
| | ppgfpgapgpkgeigavgnagpagpagprgevglpglsgpvgppgnpgangltgakgaaglp |
| | gvagapglpgprgipgpvgaagatgarglvgepgpagskgesgnkgepgsagpqgppgpsge |
| | egkrgpngeagsagppgppglrgspgsrglpgadgragvmgppgsrgasgpagvrgpngdag |
| | rpgepglmgprglpgspgnigpagkegpvglpgidgrpgpigpagargepgnigfpgpkgptg |
| | dpgkngdkghaglagargapgpdgnngaqgppgpqgvqggkgeqgppgppgfqglpgpsg |
| | pagevgkpgerglhgefglpgpagprgergppgesgaagptgpigsrgpsgppgpdgnkgepg |
| | vvgavgtagpsgpsglpgergaagipggkgekgepglrgeignpgrdgargapgavgapgpag |
| | atgdrgeagaagpagpagprgspgergevgpagpngfagpagaagqpgakgergakgpkgen |
| | gvvgptgpvgaagpagpngppgpagsrgdggppgmtgfpgaagrtgppgpsgisgppgppg |
| | pagkeglrgprgdqgpvgrtgevgavgppgfagekgpsgeagtagppgtpgpqgllgapgilgl |
| | pgsrgerglpgvagavgepgplgiagppgargppgavgspgvngapgeagrdgnpgndgppg |
| | rdgqpghkgergypgnigpvgaagapgphgpvgpagkhgnrgetgpsgpvgpagavgprgp |
| | sgpqgirgdkgepgekgprglpglkghnglqglpgiaghhgdqgapgsvgpagprgpagpsgp |
| | agkdgrtghpgtvgpagirgpqghqgpagppgppgppgppgvsgggydfgydgdfyradqpr |
| | sapslrpkdyevdatlkslnnqietlltpegsrknpartcrdlrlshpewssgyywidpnqgctmda |
| | ikvycdfstgetciraqpenipaknwyrsskdkkhvwlgetinagsqfeynvegvtskematqla |
| | fmrllanyasqnityhcknsiaymdeetgnlkkavilqgsndvelvaegnsrftytvlvdgcskkt |
| | newgktiieyktnkpsrlpfldiapldiggadgeffvdigpvcfk |
| Collagen α1(II) | mirlgapqtlvlltllvaavlrcqgqdvqeagscvqdgqryndkdvwkpepcricvcdtgtvlcdd |
| (SEQ ID NO: 24) | iicedvkdclspeipfgeccpicptdlatasgqpgpkgqkgepgdikdivgpkgppgpqgpage |
| | qgprgdrgdkgekgapgprgrdgepgtpgnpgppgppgppgppglggnfaaqmaggfdeka |
| | ggaqlgvmqgpmgpmgprgppgpagapgpqgfqgnpgepgepgvsgpmgprgppgppg |
| | kpgddgeagkpgkagergppgpqgargfpgtpglpgvkghrgypgldgakgeagapgvkges |
| | gspgengspgpmgprglpgergrtgpagaagargndgqpgpagppgpvgpaggpgfpgapg |
| | akgeagptgargpegaqgprgepgtpgspgpagasgnpgtdgipgakgsagapgiagapgfpg |
| | prgppgpqgatgplgpkgqtgepgiagfkgeqgpkgepgpagpqgapgpageegkrgargep |
| | ggvgpigppgergapgnrgfpgqdglagpkgapgergpsglagpkgangdpgrpgepglpgar |
| | gltgrpgdagpqgkvgpsgapgedgrpgppgpqgargqpgvmgfpgpkgangepgkagek |
| | glpgapglrglpgkdgetgaagppgpagpagergeqgapgpsgfqglpgppgppgeggkpgd |
| | qgvpgeagapglvgprgergfpgergspgaqglqgprglpgtpgtdgpkgasgpagppgaqgp |
| | pglqgmpgergaagiagpkgdrgdvgekgpegapgkdggrgltgpigppgpagangekgevg |
| | ppgpagsagargapgergetgppgpagfagppgadgqpgakgeqgeagqkgdagapgpqgp |
| | sgapgpqgptgvtgpkgargaqgppgatgfpgaagrvgppgsngnpgppgppgpsgkdgpk |
| | gargdsgppgragepglqgpagppgekgepgddgpsgaegppgpqglagqrgivglpgqrger |
| | gfpglpgpsgepgkqgapgasgdrgppgpvgppgltgpagepgregspgadgppgrdgaagv |
| | kgdrgetgavgapgapgppgspgpagptgkqgdrgeagaqgpmgpsgpagargiqgpqgpr |
| | gdkgeagepgerglkghrgftglqglpgppgpsgdqgasgpagpsgprgppgpvgpsgkdga |
| | ngipgpigppgprgrsgetgpagppgnpgppgppgppgpgidmsafaglgprekgpdplqym |
| | radqaagglrqhdaevdatlkslnnqiesirspegsrknpartcrdlklchpewksgdywidpnqg |
| | ctldamkvfcnmetgetcvypnpanvpkknwwsskskekkhiwfgetinggfhfsygddnla |
| | pntanvqmtflrllstegsqnityhcknsiayldeaagnlkkalliqgsndveiraegnsrftytalkd |
| | gctkhtgkwgktvieyrsqktsrlpiidiapmdiggpegefgvdigpvcfl |
| Collagen α1(III) | mmsfvqkgswlllallhptiilaqqeaveggcshlgqsyadrdvwkpepcqicvcdsgsvlcddi |
| (SEQ ID NO: 25) | icddqeldcpnpeipfgeccavcpqpptaptrppngqgpqgpkgdpgppgipgrngdpgipgq |
| | pgspgspgppgicescptgpqnyspqydsydvksgvavgglagypgpagppgppgppgtsgh |
| | pgspgspgyqgppgepgqagpsgppgppgaigpsgpagkdgesgrpgrpgerglpgppgikg |
| | pagipgfpgmkghrgfdgrngekgetgapglkgenglpgengapgpmgprgapgergrpglp |
| | gaagargndgargsdgqpgppgppgtagfpgspgakgevgpagspgsngapgqrgepgpqg |
| | hagaqgppgppgingspggkgemgpagipgapglmgargppgpagangapglrggagepg |
| | kngakgepgprgergeagipgvpgakgedgkdgspgepganglpgaagergapgfrgpagpn |
| | gipgekgpagergapgpagprgaagepgrdgvpggpgmrgmpgspggpgsdgkpgppgsq |
| | gesgrpgppgpsgprgqpgvmgfpgpkgndgapgkngerggpggpgpqgppgkngetgpq |
| | gppgptgpggdkgdtgppgpqglqglpgtggppgengkpgepgpkgdagapgapggkgdag |
| | apgergppglagapglrggagppgpeggkgaagppgppgaagtpglqgmpgergglgspgpk |
| | gdkgepggpgadgvpgkdgprgptgpigppgpagqpgdkgeggapglpgiagprgspgerge |
| | tgppgpagfpgapgqngepggkgergapgekgeggppgvagppggsgpagppgpqgvkge |
| | rgspggpgaagfpgarglpgppgsngnpgppgpsgspgkdgppgpagntgapgspgvsgpk |
| | gdagqpgekgspgaqgppgapgplgiagitgarglagppgmpgprgspgpqgvkgesgkpga |
| | nglsgergppgpqglpglagtagepgrdgnpgsdglpgrdgspggkgdrgengspgapgapgh |
| | pgppgpvgpagksgdrgesgpagpagapgpagsrgapgpqgprgdkgetgergaagikghrg |
| | fpgnpgapgspgpagqqgaigspgpagprgpvgpsgppgkdgtsghpgpigppgprgnrger |
| | gsegspghpgqpgppgppgapgpccggvgaaaiagiggekaggfapyygdepmdfkintdei |
| | mtslksvngqieslispdgsrknparnerdlkfchpelksgeywvdpnqgckldaikvfcnmetg |
| | etcisanplnvprkhwwtdssaekkhvwfgesmdggfqfsygnpelpedvldvhlaflrllssra |
| | sqnityhcknsiaymdqasgnvkkalklmgsnegefkaegnskftytvledgctkhtgewsktvf |
| | eyrtrkavrlpivdiapydiggpdgefgvdvgpvcfl |
| Collagen α1(IV) | mgprlsvwllllpaalllheehsraaakggcagsgcgkcdchgvkgqkgerglpglqgvigfpg |
| (SEQ ID NO: 26) | mqgpegpqgppgqkgdtgepglpgtkgtrgppgasgypgnpglpgipgqdgppgppgipgc |
| | ngtkgergplgppglpgfagnpgppglpgmkgdpgeilghvpgmllkgergfpgipgtpgppg |
| | lpglqgpvgppgftgppgppgppgppgekgqmglsfqgpkgdkgdqgvsgppgvpgqaqv |
| | qekgdfatkgekgqkgepgfqgmpgvgekgepgkpgprgkpgkdgdkgekgspgfpgepg |
| | ypgligrqgpqgekgeagppgppgivigtgplgekgergypgtpgprgepgpkgfpglpgqpg |
| | ppglpvpgqagapgfpgergekgdrgfpgtslpgpsgrdglpgppgspgppgqpgytngivec |
| | qpgppgdqgppgipgqpgfigeigekgqkgesclicdidgyrgppgpqgppgeigfpgqpgak |
| | gdrglpgrdgvagvpgpqgtpgligqpgakgepgefyfdlrlkgdkgdpgfpgqpgmpgrags |
| | pgrdghpglpgpkgspgsvglkgergppggvgfpgsrgdtgppgppgygpagpigdkgqagf |
| | pggpgspglpgpkgepgkivplpgppgaeglpgspgfpgpqgdrgfpgtpgrpglpgekgavg |
| | qpgigfpgppgpkgvdglpgdmgppgtpgrpgfnglpgnpgvqgqkgepgvglpglkglpgl |
| | pgipgtpgekgsigvpgvpgehgaigppglqgirgepgppglpgsvgspgvpgigppgargpp |
| | ggqgppglsgppgikgekgfpgfpgldmpgpkgdkgaqglpgitgqsglpglpgqqgapgipg |
| | fpgskgemgvmgtpgqpgspgpvgapglpgekgdhgfpgssgprgdpglkgdkgdvglpgk |
| | pgsmdkvdmgsmkgqkgdqgekgqigpigekgsrgdpgtpgvpgkdgqagqpgqpgpkg |
| | dpgisgtpgapglpgpkgsvggmglpgtpgekgvpgipgpqgspglpgdkgakgekgqagp |
| | pgigipglrgekgdqgiagfpgspgekgekgsigipgmpgspglkgspgsvgypgspglpgek |
| | gdkglpgldgipgvkgeaglpgtpgptgpagqkgepgsdgipgsagekgepglpgrgfpgfpga |
| | kgdkgskgevgfpglagspgipgskgeqgfmgppgpqgqpglpgspghategpkgdrgpqg |
| | qpglpglpgpmgppglpgidgvkgdkgnpgwpgapgvpgpkgdpgfqgmpgiggspgitg |
| | skgdmgppgvpgfqgpkglpglqgikgdqgdqgvpgakglpgppgppgpydiikgepglpg |
| | pegppglkglqglpgpkgqqgvtglvgipgppgipgfdgapgqkgemgpagptgprgfpgpp |
| | gpdglpgsmgppgtpsvdhgflvtrhsqtiddpqcpsgtkilyhgysllyvqgnerahgqdlgta |
| | gsclrkfstmpflfcninnvcnfasrndysywlstpepmpmsmapitgenirpfisrcavceapa |
| | mvmavhsqtiqippcpsgwsslwigysfvmhtsagaegsgqalaspgscleefrsapfiechgr |
| | gtcnyyanaysfwlatiersemfkkptpstlkagelrthvsrcqvcmrrt |
| Collagen α2(IV) | mgrdqravagpalrrwlllgtvtvgflaqsvlagvkkfdvpcggrdcsggcqcypekggrgqpg |
| (SEQ ID NO: 27) | pvgpqgyngppglqgfpglqgrkgdkgergapgvtgpkgdvgargvsgfpgadgipghpgqg |
| | gprgrpgydgcngtqgdsgpqgppgsegftgppgpqgpkgqkgepyalpkeerdryrgepge |
| | pglvgfqgppgrpghvgqmgpvgapgrpgppgppgpkgqqgnrglgfygvkgekgdvgqp |
| | gpngipsdtlhpiiaptgvtfhpdqykgekgsegepgirgislkgeegimgfpglrgypglsgekg |
| | spgqkgsrgldgyqgpdgprgpkgeagdpgppglpaysphpslakgargdpgfpgaqgepgs |
| | qgepgdpglpgppglsigdgdqrrglpgemgpkgfigdpgipalyggppgpdgkrgppgppgl |
| | pgppgpdgflfglkgakgragfpglpgspgargpkgwkgdagecrctegdeaikglpglpgpkg |
| | fagingepgrkgdrgdpgqhglpgfpglkgvpgnigapgpkgakgdsrtittkgergqpgvpgv |
| | pgmkgddgspgrdgldgfpglpgppgdgikgppgdpgypgipgtkgtpgemgppglglpglk |
| | gqrgfpgdaglpgppgflgppgpagtpgqidcdtdvkravggdrqeaiqpgciggpkglpglpg |
| | ppgptgakglrgipgfagadggpgprglpgdagregfpgppgfigprgskgavglpgpdgspgp |
| | iglpgpdgppgerglpgevlgaqpgprgdagvpgqpglkglpgdrgppgfrgsqgmpgmpgl |
| | kgqpglpgpsgqpglygppglhgfpgapgqegplglpgipgreglpgdrgdpgdtgapgpvg |
| | mkglsgdrgdagftgeqghpgspgfkgidgmpgtpglkgdrgspgmdgfqgmpglkgrpgf |
| | pgskgeagffgipglkglagepgfkgsrgdpgppgpppvilpgmkdikgekgdegpmglkgyl |
| | gakgiqgmpgipglsgipglpgrpghikgvkgdigvpgipglpgfpgvagppgitgfpgfigsrg |
| | dkgapgraglygeigatgdfgdigdtinlpgrpglkgergttgipglkgffgekgtegdigfpgitgv |
| | tgvqgppglkgqtgfpgltgppgsqgelgriglpggkgddgwpgapglpgfpglrgirglhglpgt |
| | kgfpgspgsdihgdpgfpgppgergdpgeantlpgpvgvpgqkgdqgapgergppgspglqg |
| | fpgitppsnisgapgdkgapgifglkgyrgppgppgsaalpgskgdtgnpgapgtpgtkgwagd |
| | sgpqgrpgvfglpgekgprgeqgfmgntgptgavgdrgpkgpkgdpgfpgapgtvgapgiagi |
| | pqkiavqpgtvgpqgrrgppgapgemgpqgppgepgfrgapgkagpqgrggvsavpgfrgd |
| | egpighqgpigqegapgrpgspglpgmpgrsvsigyllvkhsqtdqepmcpvgmnklwsgys |
| | llyfegqekahnqdlglagsclarfstmpflycnpgdvcyyasrndksywlsttaplpmmpvaed |
| | eikpyisrcsvceapaiaiavhsqdvsiphcpagwrslwigysflmhtaagdegggqslvspgscl |
| | edfratpfiecnggrgtchyyankysfwlttipegsfqgspsadtlkaglirthisrcqvcmknl |
| Collagen α1(V) | mdvhtrwkarsalrpgapllppllllllwapppsraaqpadllkvldfhnlpdgitkttgfcatrrssk |
| (SEQ ID NO: 28) | gpdvayrvtkdaqlsaptkqlypasafpedfsilttvkakkgsqaflvsiyneqgiqqiglelgrspv |
| | flyedhtgkpgpedyplfrginlsdgkwhrialsvhkknvtlildckkkttkfldrsdhpmidingii |
| | vfgtrildeevfegdiqqllfvsdhraaydycehyspdcdtavpdtpqsqdpnpdeyytegdgeg |
| | etyyyeypyyedpedlgkeptpskkpveaakettevpeeltptpteaapmpetsegagkeedvgi |
| | gdydyvpsedyytpspyddltygegeenpdqptdpgagaeiptstadtsnssnpapppgegadd |
| | legefteetirnldenyydpyydptsspseigpgmpanqdtiyegiggprgekgqkgepaiiepg |
| | mliegppgpegpaglpgppgtmgptgqvgdpgergppgrpglpgadglpgppgtmlmlpfrf |
| | ggggdagskgpmvsaqesqaqailqqarlalrgpagpmgltgrpgpvgppgsgglkgepgdvg |
| | pqgprgvqgppgpagkpgrrgragsdgargmpgqtgpkgdrgfdglaglpgekghrgdpgps |
| | gppgppgddgergddgevgprglpgepgprgllgpkgppgppgppgvtgmdgqpgpkgnv |
| | gpqgepgppgqqgnpgaqglpgpqgaigppgekgplgkpglpgmpgadgppghpgkegpp |
| | gekggqgppgpqgpigypgprgvkgadgirglkgtkgekgedgfpgfkgdmgikgdrgeigp |
| | pgprgedgpegpkgrggpngdpgplgppgekgklgvpglpgypgrqgpkgsigfpgfpgang |
| | ekggrgtpgkpgprgqrgptgprgergprgitgkpgpkgnsggdgpagppgergpngpqgptg |
| | fpgpkgppgppgkdglpghpgqrgetgfqgktgppgppgvvgpqgptgetgpmgerghpgp |
| | pgppgeqglpglagkegtkgdpgpaglpgkdgppglrgfpgdrglpgpvgalglkgnegppgp |
| | pgpagspgergpagaagpigipgrpgpqgppgpagekgapgekgpqgpagrdglqgpvglpg |
| | pagpvgppgedgdkgeigepgqkgskgdkgeqgppgptgpqgpigqpgpsgadgepgprgq |
| | qglfgqkgdegprgfpgppgpvglqglpgppgekgetgdvgqmgppgppgprgpsgapgad |
| | gpqgppggignpgavgekgepgeagepglpgeggppgpkgergekgesgpsgaagppgpkg |
| | ppgddgpkgspgpvgfpgdpgppgepgpagqdgppgdkgddgepgqtgspgptgepgpsg |
| | ppgkrgppgpagpegrqgekgakgeaglegppgktgpigpqgapgkpgpdglrgipgpvgeq |
| | glpgspgpdgppgpmgppglpglkgdsgpkgekghpgligligppgeqgekgdrglpgpqgs |
| | sgpkgeqgitgpsgpigppgppglpgppgpkgakgssgptgpkgeaghpgppgppgppgevi |
| | qplpiqasrtrrnidasqllddgngenyvdyadgmeeifgslnslkleieqmkrplgtqqnpartck |
| | dlqlchpdfpdgeywvdpnqgcsrdsfkvycnftaggstcvfpdkksegaritswpkenpgswf |
| | sefkrgkllsyvdaegnpvgvvqmtflrllsasahqnvtyhcyqsvawqdaatgsydkalrflgsn |
| | deemsydnnpyiralvdgcatkkgyqktvleidtpkveqvpivdimfndfgeasqkfgfevgpa |
| | cfmg |

Studies have documented the capacity of extracellular matrix (ECM) proteins containing the short amino acid sequence RGD to support interactions mediated by integrin receptors. The ability of cells to interact with RGD sites within the context of larger glycoproteins depends on many factors, some of which include the adjacent flanking sequences surrounding the core RGD tri-peptide as well as the geometrical configuration of the intact molecule and how these molecules are oriented within the context of the interconnected network of other ECM proteins. Flanking sequences immediately C-terminal to the RGD site can govern integrin selective binding. RGD motifs can be cryptic and inaccessible to cell surface receptors as is illustrated in the case of triple helical collagen. In this regard, five different cryptic RGD containing sites exist within human collagen type-I, each with distinct flanking sequences (Table 3).

Exemplary cryptic collagen epitope and blocking peptides (also known as mimicking peptides), which block antibodies from binding cryptic collagen epitopes, are shown in Table 3.

TABLE 3

| SEQ ID NO | Sequence |
|---|---|
| SEQ ID NO: 1 | RGDKGE |
| SEQ ID NO: 2 | KGDRGDAPG |
| SEQ ID NO: 3 | QGPRGDKGE |
| SEQ ID NO: 4 | GPOG |
| SEQ ID NO: 5 | GPOGPOP |
| SEQ ID NO: 6 | GPPG |
| SEQ ID NO: 7 | GPPGPPG |
| SEQ ID NO: 12 | AGSRGDGGP |
| SEQ ID NO: 13 | QG1RGDKGE |
| SEQ ID NO: 14 | RGPRGDQGP |
| SEQ ID NO: 15 | QGPSGAPGE |
| SEQ ID NO: 16 | CPGFPGFC |
| SEQ ID NO: 17 | PGAKGLPGPPGPPGPY |
| SEQ ID NO: 18 | GFOGIOGTOGPOGLO |
| SEQ ID NO: 19 | GEXGDQGIAGFOGSO |
| SEQ ID NO: 20 | GPQGQPGLOGLOGPM |
| SEQ ID NO: 21 | GFOGIOT |
| SEQ ID NO: 22 | GDTGPOGPOGY |

Five different cryptic RGD containing sites exist within human collagen type-I, each with distinct flanking sequences. Additionally, a control peptide (P-C) was generated lacking the RGD tri-peptide motif. The sequences in Table 2 correspond to the following SEQ ID NOs.: KGDRGDAPG (SEQ ID NO: 2), QGPRGDKGE (SEQ ID NO: 3), AGSRGDGGP (SEQ ID NO: 12), QGIRGDKGE (SEQ ID NO 13); RGPRGDQGP (SEQ ID NO: 14); and QGPSGAPGE (SEQ ID NO: 15).

Cryptic CPGFPGFC (SEQ ID NO: 16)-containing collagen epitope is HU177 epitope. HU177 also has been shown to bind the following amino acid sequences: GPOG (SEQ ID NO: 4); GPOGPOP (SEQ ID NO: 5); GPPG (SEQ ID NO: 6); GPPGPPG (SEQ ID NO: 7); PGAKGLPGPPGPPGPY (SEQ ID NO: 17); GFOGIOGTOGPOGLO (SEQ ID NO: 18); GEXGDQGIAGFOGSO (SEQ ID NO: 19); GPQGQPGLOGLOGPM (SEQ ID NO: 20); GFOGIOT (SEQ ID NO: 21); and GDTGPOGPOGY (SEQ ID NO: 22).

### Methods of Treatment

In another aspect of the present invention, the disclosure provides a method of treating cancer in a subject, the method comprising administering to the subject a therapeutically effective amount of the ADC of the above aspects or any other aspects of the invention delineated herein, or the pharmaceutical composition of the above aspects or any other aspects of the invention delineated herein, thereby treating the cancer.

In another aspect of the present invention, the disclosure provides a method for inhibiting or decreasing solid tumor growth in a subject having a solid tumor, the method comprising administering to the subject a therapeutically effective amount of the ADC of the above aspects or any other aspects of the invention delineated herein, or the pharmaceutical composition of the above aspects or any other aspects of the invention delineated herein, thereby the solid tumor growth is inhibited or decreased.

In another aspect of the present invention, the disclosure provides a method of delivering a drug to a cancer cell in a subject, the method comprising administering to the subject a therapeutically effective amount of the ADC of the above aspects or any other aspects of the invention delineated herein, or the pharmaceutical composition of the above aspects or any other aspects of the invention delineated herein.

In some embodiments, the disease or disorder is an autoimmune disease or cancer.

In some embodiments, the methods further comprise inhibiting an inflammatory disease or disorder. Exemplary inflammatory diseases or disorders include, but are not limited to, an allergy, ankylosing spondylitis, asthma, atopic dermatitis, an autoimmune disease or disorder, a cancer, celiac disease, chronic obstructive pulmonary disease (COPD), chronic peptic ulcer, cystic fibrosis, diabetes, glomerulonephritis, gout, hepatitis, an immune-mediated disease or disorder, inflammatory bowel disease (IBD), myositis, osteoarthritis, pelvic inflammatory disease (PID), multiple sclerosis, neurodegenerative diseases of aging, a periodontal disease, reperfusion injury transplant rejection, psoriasis, pulmonary fibrosis, rheumatic disease, scleroderma, sinusitis, dermatitis, pneumonitis, colitis and tuberculosis.

By “autoimmune disease” is meant a disease characterized by a dysfunction in the immune system. The disease is characterized by the components of the immune system affected, whether the immune system is overactive or underactive, or whether the condition is congenital or acquired. In most cases, the disorder causes abnormally low activity or over activity of the immune system. In cases of immune system over activity, the body attacks and damages its own tissues (autoimmune). Immune deficiency diseases decrease the body's ability to fight invaders, causing vulnerability to infections. In response to an unknown trigger, the immune system may begin producing antibodies that instead of fighting infections, attack the body's own tissues. Treatment for autoimmune diseases generally focuses on reducing immune system activity. Exemplary autoimmune diseases include, but are not limited to, Psoriasis, Graft-vs-Host Disease, Amyotrophic Lateral Sclerosis, Pemphigus Vulgaris, Systemic Lupus Erythematosus, Scleroderma, Ulcerative Colitis, Crohn's Disease, Type 1 Diabetes, Multiple Sclerosis, Alopecia Areata, Uveitis, Neuromyelitis Optica, Graves' disease, Hashimoto's thyroiditis, rheumatoid arthritis and Duchenne Muscular Dystrophy.

By “cancer” is meant, comprising of but not limited to melanoma, central nervous system (CNS) cancer, CNS germ cell tumor, lung cancer, leukemia, multiple myeloma, renal cancer, malignant glioma, medulloblatoma, breast cancer, ovarian cancer, prostate cancer, bladder cancer, fibrosarcoma, pancreatic cancer, gastric cancer, head and neck cancer, colorectal cancer. For example, a cancer cell is derived from a solid cancer or hematological cancer. The hematological cancer is, e.g., a leukemia or a lymphoma. A leukemia is acute lymphoblastic leukemia (ALL), acute myelogenous leukemia (AML), chronic lymphocytic leukemia (CLL), small lymphocytic lymphoma (SLL), chronic myelogenous leukemia (CML), or acute monocytic leukemia (AMOL). A lymphoma is follicular lymphoma, Hodgkin's lymphoma (e.g., Nodular sclerosing subtype, mixed-cellularity subtype, lymphocyte-rich subtype, or lymphocyte depleted subtype), or Non-Hodgkin's lymphoma. Exemplary solid cancers include but are not limited to melanoma (e.g., unresectable, metastatic melanoma), renal cancer (e.g., renal cell carcinoma), prostate cancer (e.g., metastatic castration resistant prostate cancer), ovarian cancer (e.g., epithelial ovarian cancer, such as metastatic epithelial ovarian cancer), breast cancer (e.g., triple negative breast cancer), and lung cancer (e.g., non-small cell lung cancer).

By “diagnosing” and the like as used herein refers to a clinical or other assessment of the condition of a subject based on observation, testing, or circumstances for identifying a subject having a disease, disorder, or condition based on the presence of at least one indicator, such as a sign or symptom of the disease, disorder, or condition. Typically, diagnosing using the method of the invention includes the observation of the subject for multiple indicators of the disease, disorder, or condition in conjunction with the methods provided herein. Diagnostic methods provide an indicator that a disease is or is not present. A single diagnostic test typically does not provide a definitive conclusion regarding the disease state of the subject being tested.

By the terms “effective amount” and “therapeutically effective amount” of a formulation or formulation component is meant a sufficient amount of the formulation or component, alone or in a combination, to provide the desired effect. For example, by “an effective amount” is meant an amount of a compound, alone or in a combination, required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

Human dosage amounts can initially be determined by extrapolating from the amount of compound used in mice or nonhuman primates, as a skilled artisan recognizes it is routine in the art to modify the dosage for humans compared to animal models. In certain embodiments, it is envisioned that the dosage of the antagonist to collagen may vary from between about 0.1 μg compound/kg body weight to about 25000 μg compound/kg body weight; or from about 1 μg/kg body weight to about 4000 μg/kg body weight or from about 10 μg/kg body weight to about 3000 μg/kg body weight. In other embodiments this dose may be about 0.1, 0.3, 0.5, 1, 3, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, 5000, 5500, 6000, 6500, 7000, 7500, 8000, 8500, 9000, 9500, 10000, 10500, 1100, 11500, 12000, 12500, 13000, 13500, 14000, 14500, 15000, 15500, 16000, 16500, 17000, 17500, 18000, 18500, 19000, 19500, 20000, 20500, 21000, 21500, 22000, 22500, 23000, 23500, 24000, 24500, or 25000 μg/kg body weight. In other embodiments, it is envisaged that doses may be in the range of about 0.5 μg compound/kg body weight to about 20 μg compound/kg body weight. In other embodiments, the doses may be about 0.5, 1, 3, 6, 10, or 20 mg/kg body weight. Of course, this dosage amount may be adjusted upward or downward, as is routinely done in such treatment protocols, depending on the results of the initial clinical trials and the needs of a particular patient.

The compositions of the invention (e.g., antibody drug conjugate) are administered once per month, twice per month (i.e., every two weeks), every week, once per day, twice per day, every 12 hours, every 8 hours, every 4 hours, every 2 hours or every hour. The compositions of the invention (e.g., antibody drug conjugate) are administered for a duration of 1 day, 2 days, 3 days, 4 days, 5 days, 6 days, 7 days, 2 weeks, 3 weeks, 4 weeks, five weeks, six weeks, 2 months, 3 months, 4 months, 5 months, 6 months, 7 months, 8 months, 9 months, 10 months, 11 months, 12 months, 2 years, 3 years, 4 years, 5 years or more. For example, the composition of the invention (e.g., antibody drug conjugate) are administered one dose every two weeks for 4 to 6 weeks or until the disease is treated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated. Therapeutic effect may be assessed by monitoring the symptoms of a patient, tumor markers in blood or for example a size of a tumor or the length of survival of the patient.

A “subject” as used herein refers to an organism. In certain embodiments, the organism is an animal. In certain embodiments, the subject is a living organism. In certain embodiments, the subject is a cadaver organism. In certain preferred embodiments, the subject is a mammal, including, but not limited to, a human or non-human mammal. In certain embodiments, the subject is a domesticated mammal or a primate including a non-human primate. Examples of subjects include humans, monkeys, dogs, cats, mice, rats, cows, horses, goats, and sheep. A human subject may also be referred to as a patient.

A “sample” as used herein refers to a biological material that is isolated from its environment (e.g., blood or tissue from an animal, cells, or conditioned media from tissue culture) and is suspected of containing, or known to contain an analyte, such as a protein. A sample can also be a partially purified fraction of a tissue or bodily fluid. A reference sample can be a “normal” sample, from a donor not having the disease or condition fluid, or from a normal tissue in a subject having the disease or condition. A reference sample can also be from an untreated donor or cell culture not treated with an active agent (e.g., no treatment or administration of vehicle only). A reference sample can also be taken at a “zero time point” prior to contacting the cell or subject with the agent or therapeutic intervention to be tested or at the start of a prospective study.

A “subject sample” can be a sample obtained from any subject, typically a blood or serum sample, however the method contemplates the use of anybody fluid or tissue from a subject. The sample may be obtained, for example, for diagnosis of a specific individual for the presence or absence of a particular disease or condition.

A subject “suffering from or suspected of suffering from” a specific disease, condition, or syndrome has a sufficient number of risk factors or presents with a sufficient number or combination of signs or symptoms of the disease, condition, or syndrome such that a competent individual would diagnose or suspect that the subject was suffering from the disease, condition, or syndrome. Methods for identification of subjects suffering from or suspected of suffering from conditions associated with diminished cardiac function is within the ability of those in the art. Subjects suffering from, and suspected of suffering from, a specific disease, condition, or syndrome are not necessarily two distinct groups.

As used herein, “susceptible to” or “prone to” or “predisposed to” a specific disease or condition and the like refers to an individual who based on genetic, environmental, health, and/or other risk factors is more likely to develop a disease or condition than the general population. An increase in likelihood of developing a disease may be an increase of about 10%, 20%, 50%, 100%, 150%, 200%, or more.

The methods described herein can be used in conjunction with one or more chemotherapeutic or anti-neoplastic agents. In some cases, the additional chemotherapeutic agent is radiotherapy. In some cases, the chemotherapeutic agent is a cell death-inducing agent.

Also provided is a method of treating a disease characterized by abnormal immune suppression in a subject by identifying a subject, e.g., a human, that has been diagnosed with a disease characterized by abnormal immune suppression, administering an antibody drug conjugate as described herein, and administering an immune checkpoint inhibitor, thereby treating in the subject.

Suitable diseases characterized by abnormal immune suppression include Type I diabetes, lupus, psoriasis, scleroderma, hemolytic anemia, vasculitis, Graves' disease, rheumatoid arthritis, multiple sclerosis, Hashimoto's thyroiditis, Myasthenia gravis, and vasculitis.

In some embodiments, the methods described herein can be used in conjunction with an immune checkpoint inhibitor. Importantly, while immune check point inhibitors are known to provide some anti-tumor activity in humans, this partial anti-tumor activity is only observed in a fraction of treated subjects. Previously, the identification of compounds and combination treatment strategies to enhance the efficacy of immune checkpoint inhibitors such as CTLA-4, PDL-1 and PD-1 antibodies was described (see, e.g., US Patent Publications 2017-0065716, and 2017-0240638 which are incorporated herein by reference). In some embodiments, the immune checkpoint inhibitor comprises an inhibitor of CTLA-4, PD-1, PDL-1, Lag3, LAIR1, or LAIR2 In some embodiments, the immune checkpoint inhibitor comprises a CTLA-4 antibody, a PD-1 antibody, a PDL-1 antibody, a Lag3 antibody, a LAIR1 antibody or a LAIR2 antibody.

In some cases, the immune checkpoint inhibitor (e.g., CTLA-4 antibody, a PD-1 antibody, a PDL-1 antibody, Lag3, LAIR1, or LAIR 2) is administered at a dosage of 0.01-10 mg/kg (e.g., 0.01, 0.05, 0.1, 0.5, 1, 5, or 10 mg/kg) bodyweight. For example, the PDL-1 inhibitor is administered in an amount of 0.01-30 mg (e.g., 0.01, 0.05, 0.1, 0.5, 1, 5, 10, 20, or 30 mg) per dose. In another example, the antibody is administered in the dose range of 0.1 mg/kg to 10 mg/kg of body weight. In some cases, the antagonist of integrin αvβ3 is administered at a dosage of 0.01-10 mg/kg (e.g., 0.01, 0.05, 0.1, 0.5, 1, 5, or 10 mg/kg) bodyweight. In some cases, the XL313 antibody or the HU177 antibody is administered in an amount of 0.01-30 mg (e.g., 0.01, 0.05, 0.1, 0.5, 1, 5, 10, 20, or 30 mg) per dose.

In some cases, the immune checkpoint inhibitor, e.g., the inhibitor of PDL-1, is administered at a dosage of 0.01-10 mg/kg (e.g., 0.01, 0.05, 0.1, 0.5, 1, 5, or 10 mg/kg) bodyweight. For example, the PDL-1 inhibitor is administered in an amount of 0.01-30 mg (e.g., 0.01, 0.05, 0.1, 0.5, 1, 5, 10, 20, or 30 mg) per dose. In another example, the immune checkpoint inhibitor, e.g., the anti-PD-L1 antibody, is administered in the dose range of 0.1 mg/kg to 10 mg/kg of body weight. In some cases, the XL313 antibody or the HU177 antibody is administered at a dosage of 0.01-10 mg/kg (e.g., 0.01, 0.05, 0.1, 0.5, 1, 5, or 10 mg/kg) bodyweight. For example, the XL313 antibody or the HU177 antibody is administered in an amount of 0.01-30 mg (e.g., 0.01, 0.05, 0.1, 0.5, 1, 5, 10, 20, or 30 mg) per dose. For example, the dose range of Mab XL313 or Mab HU177 is from 0.1 mg/kg to 25 mg/kg of body weight.

The compositions of the invention (e.g., antibody drug conjugate, optionally in combination with inhibitor of PDL-1) are administered once per month, twice per month (i.e., every two weeks), every week, once per day, twice per day, every 12 hours, every 8 hours, every 4 hours, every 2 hours or every hour. The compositions of the invention (e.g., antibody drug conjugate, optionally in combination with inhibitor of PDL-1) are administered for a duration of 1 day, 2 days, 3 days, 4 days, 5 days, 6 days, 7 days, 2 weeks, 3 weeks, 4 weeks, five weeks, six weeks, 2 months, 3 months, 4 months, 5 months, 6 months, 7 months, 8 months, 9 months, 10 months, 11 months, 12 months, 2 years, 3 years, 4 years, 5 years or more. For example, the composition of the invention (e.g., antibody drug conjugate, optionally in combination with inhibitor of PDL-1) are administered one dose every two weeks for 4 to 6 weeks or until the disease is treated.

Also provided is a method of treating a disease characterized by an overactive immune response (e.g., an autoimmune disease) in a subject, e.g., a human subject, that has been diagnosed with an overactive immune response by administering a peptide comprising collagen or a fragment thereof, thereby treating overactive immune response in the subject. Suitable types of collagen include collagen type-I, collagen type II, collagen type III, and collagen type-IV (e.g., the alpha 6 chain of collagen type-IV). For example, the peptide comprises RGDKGE (SEQ ID NO: 1) or CPGFPGFC (SEQ ID NO: 16).

The term “antineoplastic agent” is used herein to refer to agents that have the functional property of inhibiting a development or progression of a neoplasm in a human, particularly a malignant (cancerous) lesion, such as a carcinoma, sarcoma, lymphoma, or leukemia. Inhibition of metastasis is frequently a property of antineoplastic agents.

As used herein, “kits” are understood to contain at least one non-standard laboratory reagent for use in the methods of the invention in appropriate packaging, optionally containing instructions for use. The kit can further include any other components required to practice the method of the invention, as dry powders, concentrated solutions, or ready to use solutions. In some embodiments, the kit comprises one or more containers that contain reagents for use in the methods of the invention; such containers can be boxes, ampules, bottles, vials, tubes, bags, pouches, blister-packs, or other suitable container forms known in the art. Such containers can be made of plastic, glass, laminated paper, metal foil, or other materials suitable for holding reagents.

As used herein, “obtaining” is understood herein as manufacturing, purchasing, or otherwise coming into possession of.

### Pharmaceutically Acceptable Carriers

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Formulations of the present invention include those suitable for oral, nasal, topical, transdermal, buccal, sublingual, intramuscular, intracardiac, intraperitoneal, intrathecal, intracranial, rectal, vaginal and/or parenteral administration. The formulations may conveniently be presented in unit dosage form and may be prepared by any methods well known in the art of pharmacy. The amount of active ingredient that can be combined with a carrier material to produce a single dosage form will generally be that amount of the compound that produces a therapeutic effect.

In some cases, a composition of the invention is administered orally or systemically. Other modes of administration include rectal, topical, intraocular, buccal, intravaginal, intracisternal, intracerebroventricular, intratracheal, nasal, transdermal, within/on implants, or parenteral routes. The term “parenteral” includes subcutaneous, intrathecal, intravenous, intramuscular, intraperitoneal, or infusion. Intravenous or intramuscular routes are not particularly suitable for long-term therapy and prophylaxis. They could, however, be preferred in emergency situations. Compositions comprising a composition of the invention can be added to a physiological fluid, such as blood. Oral administration can be preferred for prophylactic treatment because of the convenience to the patient as well as the dosing schedule. Parenteral modalities (subcutaneous or intravenous) may be preferable for more acute illness, or for therapy in patients that are unable to tolerate enteral administration due to gastrointestinal intolerance, ileus, or other concomitants of critical illness. Inhaled therapy may be most appropriate for pulmonary vascular diseases (e.g., pulmonary hypertension).

### EXAMPLES

Years of research has led to critical insight into the mechanisms that control ovarian cancer (1-3), and this important understanding has helped shape the development and testing of current treatment strategies (4, 5). Despite evidence indicating that the immune system plays a role in regulating ovarian tumor growth, immune checkpoint therapy has demonstrated minimal impact on ovarian cancer (6, 7). These studies, and the limited effects of standard surgical debulking and chemotherapy illustrate the urgent need for new and more effective treatments for this devastating disease. Complicating the development of new therapies is the heterogeneity of ovarian tumors, which can be characterized by a diversity of histological subtypes, regulatory molecules, and cell surface targets (8, 9). A common control protein that regulates the behavior of cells within the tumor microenvironment is collagen, and high expression of distinct forms of collagen are correlated with poor clinical outcomes in ovarian cancer (10-12). However, developing strategies to target normal intact collagen is highly challenging given its ubiquitous expression, and its roles in normal physiological processes. To this end, a hallmark of malignant tumor progression involves remodeling of the extracellular matrix (13). Importantly, antibodies specifically directed to proteolyzed and denatured forms of collagen, have shown anti-tumor activity in animal models and in humans (14-16).

### Example 1: Selective Internalization of Anti-HU177 Mab in Different Malignant Tumor Cell Types

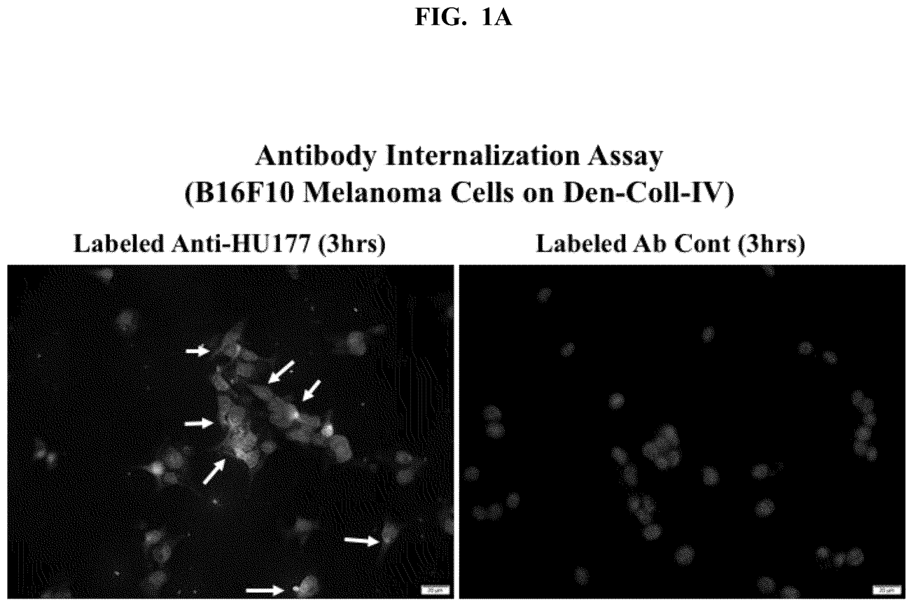

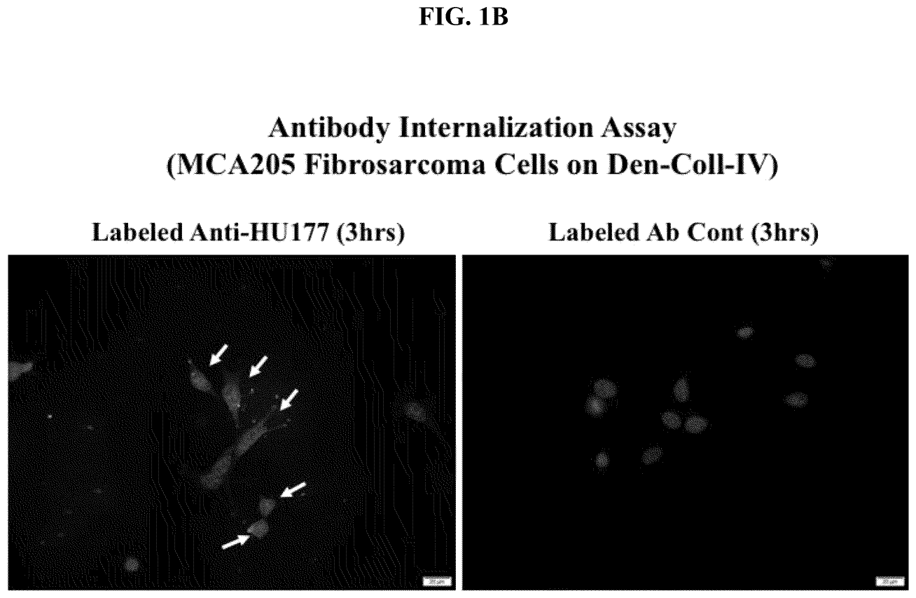

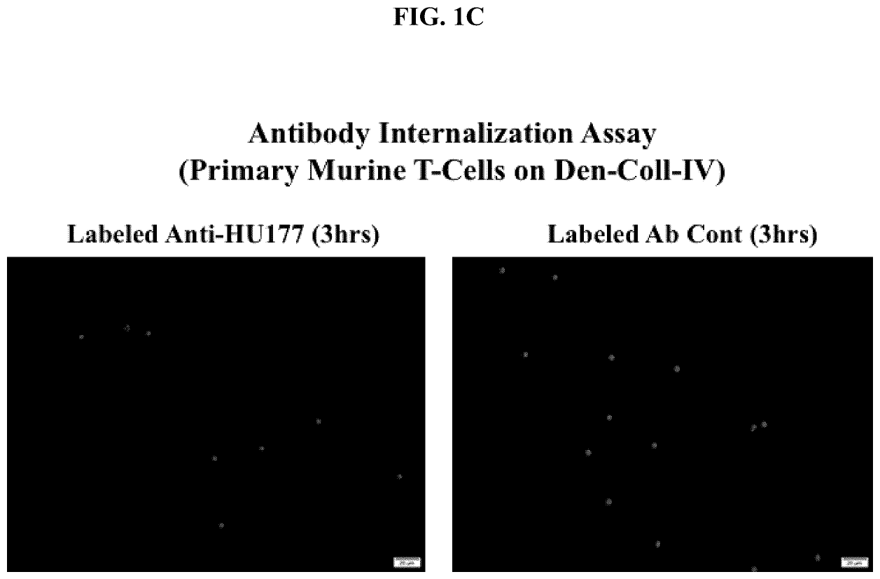

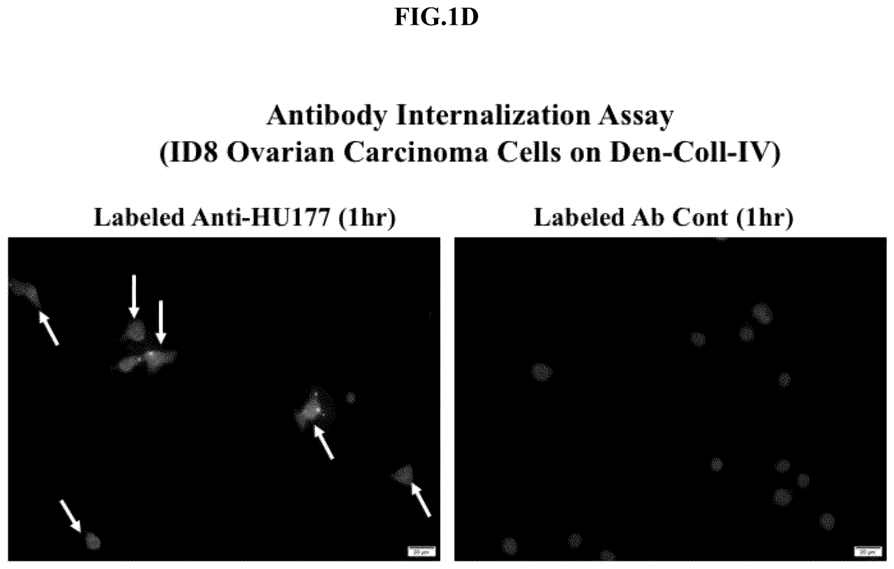

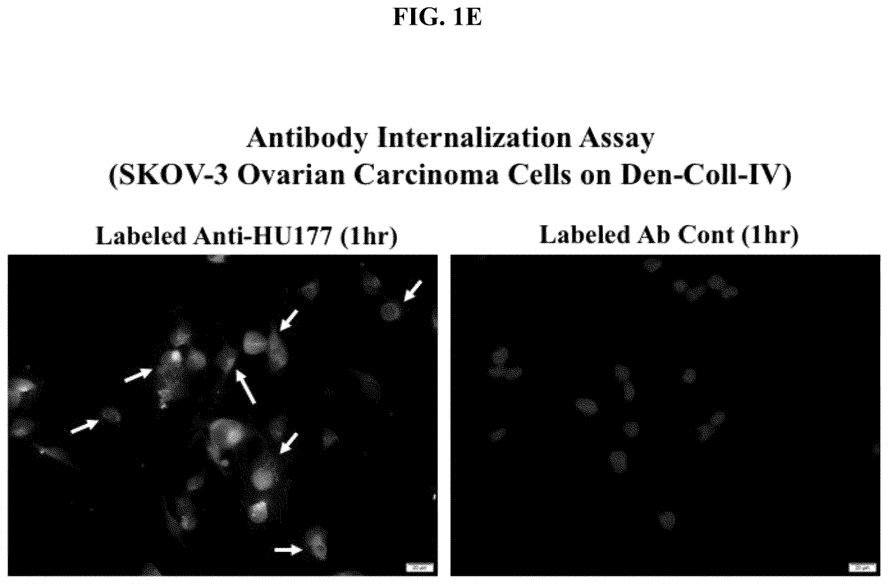

Typically an antibody drug conjugate (ADC) is composed of a cell surface-targeted antibody that is coupled to a cleavable linker which is conjugated to a therapeutic drug such as monomethyl auristatin E (MMAE) or monomethyl auristatin F(MMAF). Currently, among the most effective mechanisms by which ADCs exhibit maximum therapeutic activity, involves the ADC being internalized inside the cell, where the linker is cleaved to facilitate release of the drug inside the cell in order to promote efficient cell killing. In this regard, all ADCs currently approved selectively target cell surface molecules that facilitate the ADC internalization. Contrary to currently accepted dogma for typical ADCs, we tested whether the unique molecular composition of anti-HU177 antibody, which does not bind to a cell surface molecule, but rather selectively binds to denatured forms of the extracellular matrix protein collagen, might allow selective internalization in tumor cells. To test this possibility, we used a commercially available antibody internalization kit (pHrod iFL microscale protein labeling kit), which allows monitoring of internalization of antibodies via release of a fluorescent probe following the cleavage of a pH sensitive linker within the acidic lysosomes inside cells. B16F10 metastatic melanoma cells were mixed with anti-HU177 Mab or a non-specific control antibody, which were previously labeled with the fluorescent internalization probe. Next, cells were seeded on slides coated with denatured collagen-IV. The cells were allowed to incubate for 1 to 3 hours then analyzed by fluorescence microscopy for internalization as indicated by release of the red fluorescence probe. Surprisingly, while the non-specific control antibody labeled with the internalization probe showed little if any internalization, the anti-HU177 antibody showed clear internalization as indicated by the red fluorescence (FIG. 1A). To determine whether the anti-HU177 antibody could be internalized in other tumor cell types, we carried out similar experiments using MCA205 fibrosarcoma cells. Importantly, similar selective internalization was also observed with anti-HU177 antibody, while minimal if any control antibody was internalized (FIG. 1B). Next, we used a similar approach to examine whether the anti-HU177 antibody was internalized by normal non-malignant cells. To examine this possibility, we isolated normal primary T-cells from mice bearing B16F10 melanomas. As shown in FIG. 1C, neither the anti-HU177 nor the non-specific control antibody, showed evidence of internalization. These data are consistent with the unexpected findings that the unique anti-HU177 antibody, specifically directed to a cryptic epitope exposed within the extracellular matrix protein collagen, can be selectively internalized into tumor cells, while exhibiting little evidence of internalization into normal primary T-cells. Finally, using a similar experimental approach, we tested the ability of anti-HU177 antibody to be internalized into two additional malignant tumor cell types including murine ID8 ovarian carcinoma cells as well as human SKOV-3 ovarian carcinoma cells. As shown in FIGS. 1D and 1E, again anti-HU177 antibody but not the control non-specific antibody was readily internalized. Next, we examined whether an additional antibody that also binds to fragments of collagen might also be internalized, we labeled Mab XL166, which was previously shown to bind to a unique set of cryptic collagen epitope (6). Surprisingly, Mab XL166 labeled with the internalization probe exhibited no evidence of internalization into SKOV-3 cells (FIG. 1F). Collectively, these unexpected findings indicate for the first time that the anti-HU177 antibody, which is specifically directed to a defined cryptic epitope normally hidden within the extracellular matrix protein collagen, but surprisingly not Mab XL166 directed to a different set of cryptic collagen epitopes can be selectively internalized into malignant tumor cells.

### Example 2: Conjugation of the Anti-HU177 Antibody to the Therapeutic Payload MMAE Did not Significantly Alter its Functional Binding Activity

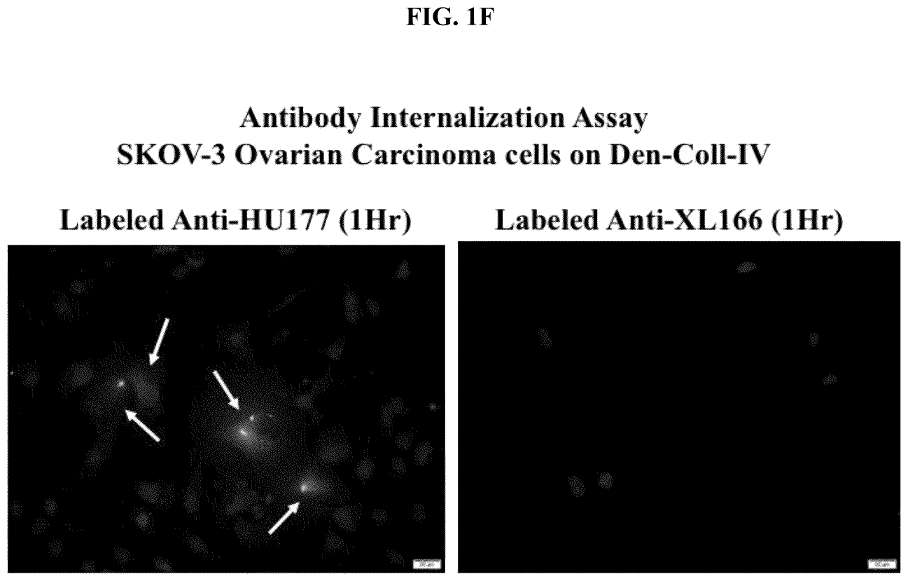

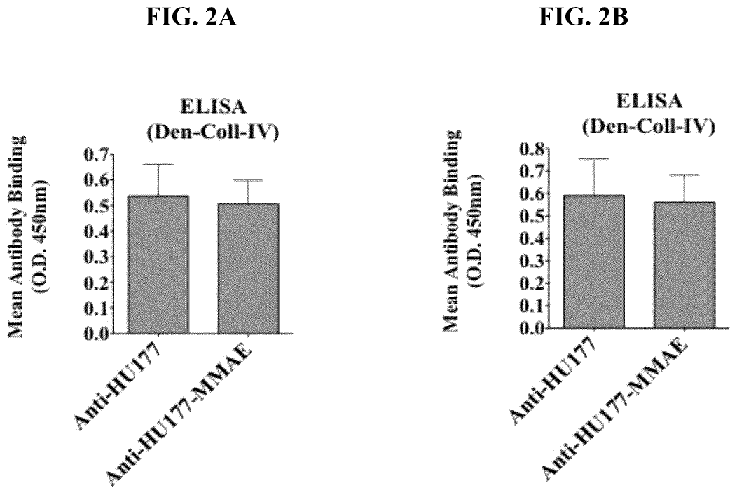

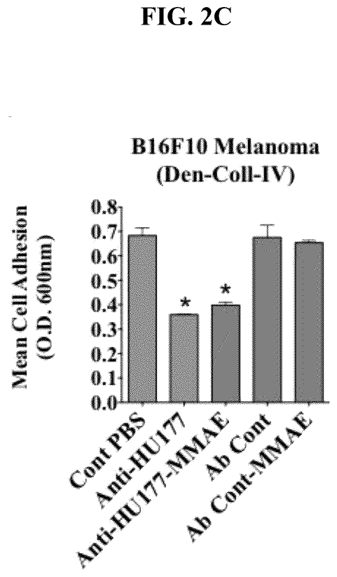

When generating novel ADCs, it is critical that the process of conjugating therapeutic payloads to the antibody does not disrupt its ability to bind to its intended target. Given our surprising findings indicating that the unique molecular composition of the anti-HU177 antibody facilitates selective internalization in malignant cells, we sought to determine whether the conjugation of a cytotoxic drug such as MMAE would alter its capacity to bind to its non-cellular target. Briefly, we first used the commercially available MMAE conjugation kit (Perkit™ Antibody MMAE Conjugation Kit) to conjugate MMAE to the anti-HU177 antibody according to the manufacturer's instructions. Using ELISA assays, we compared the binding ability of the purified unconjugated (Anti-HU177) and MMAE-conjugated anti-HU177 antibody (Anti-HU177-MMAE) to bind to denatured collagen. As shown in FIG. 2A, no significant changes were detected in the ability of 0.5 μg/ml of either the unconjugated or the anti-HU177-MMAE ADC to bind to denatured collagen. Similar results were also observed using a higher concentration of the anti-HU177 antibodies (1.0 μg/ml) (FIG. 2B). Next, we sought to determine whether conjugating MMAE to the anti-HU177 antibody had any effect on the known ability of anti-HU177 antibody to selectively inhibit tumor cell adhesion to denatured collagen. To carry out these studies we coated non-tissue culture wells with denatured collagen type-IV and compared the ability of unconjugated anti-HU177 and MMAE conjugated anti-HU177 (Anti-HU177-MMAE) to alter B16F10 melanoma cell adhesion. As shown in FIG. 2C, the unconjugated anti-HU177 antibody significantly inhibited tumor cell adhesion to denatured collagen by over 40% as compared to unconjugated or MMAE-conjugated non-specific control antibody. Importantly, the MMAE conjugated anti-HU177 antibody (Anti-HU177-MMAE) showed similar inhibitory activity, demonstrating greater than 40% inhibition of B16F10 cell adhesion as compared to controls. Collectively, these data are consistent with the concept that conjugation of MMAE to the anti-HU177 antibody does not significantly alter its ability to bind and functionally block its extracellular target.

### Example 3: The Anti-HU177-MMAE ADC Induces Morphological Changes within Several Different Malignant Tumor Cell Types

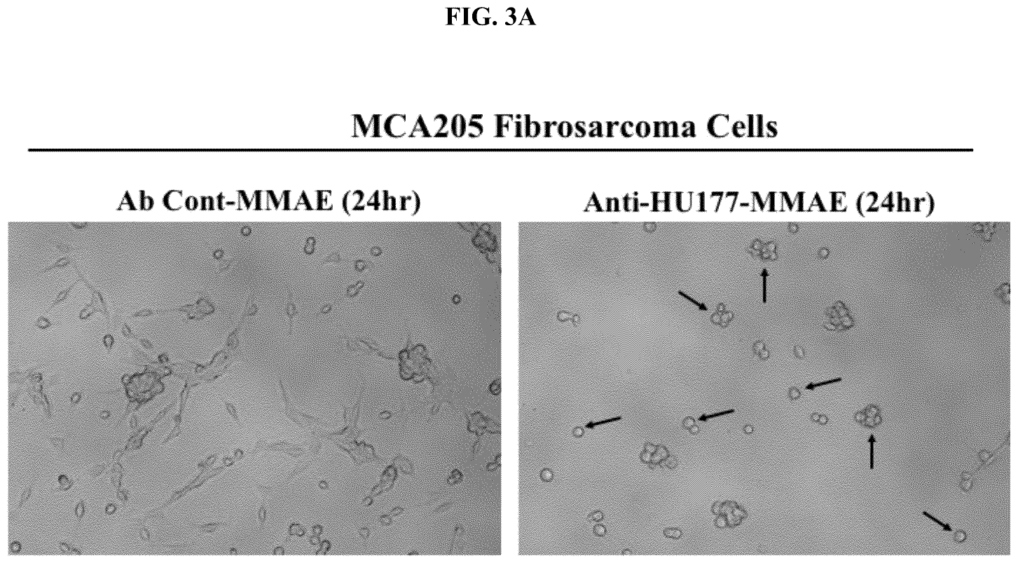

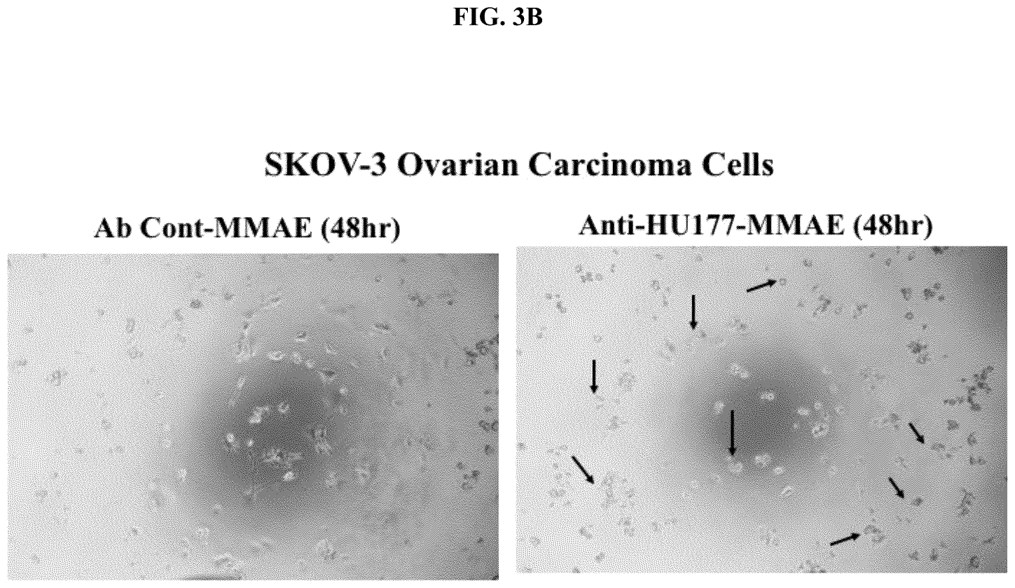

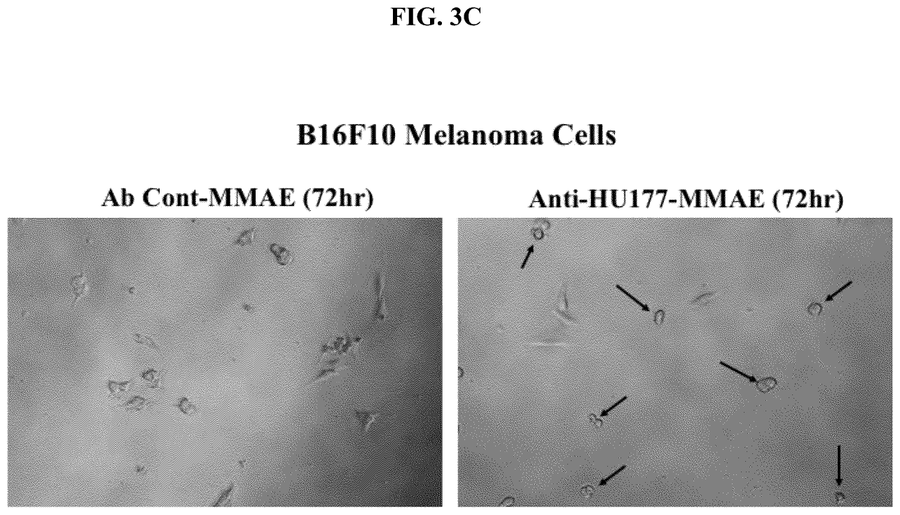

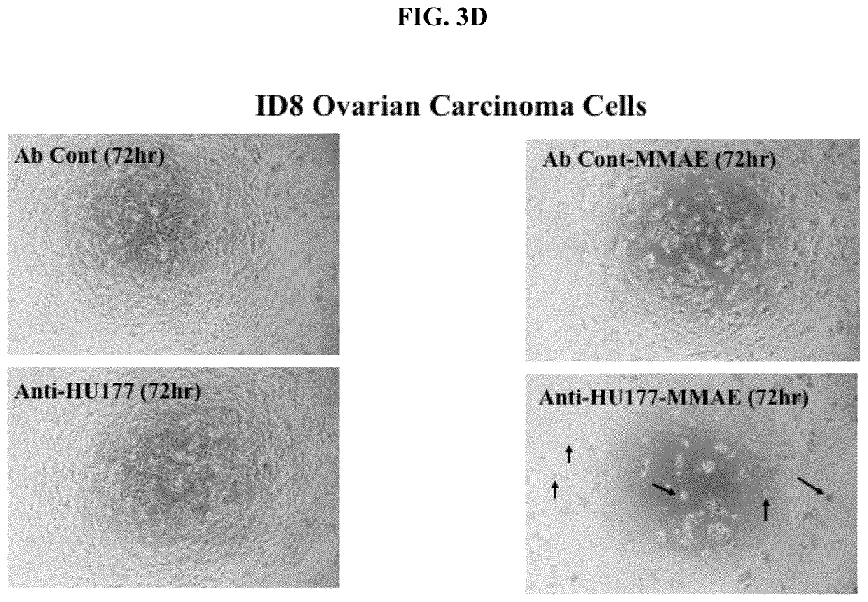

When malignant tumor cells begin to die, they may exhibit distinctive morphological changes in shape and structure, especially when the cells are treated with drugs that alter microtubule polymerization, such as MMAE and MMAF. Importantly, the ability of MMAE and MMAF to disrupt normal microtubule polymerization and induce cell death depends on the ability of the drug to bind to microtubules inside cells. Therefore, we examined the morphological changes of 4 different malignant tumor cell types over a time course following treatment with anti-HU177-MMAE and controls. Briefly, B16F10 melanoma, MCA205 fibrosarcoma, SKOV-3 ovarian carcinoma and ID8 ovarian carcinoma cells were resuspended in the presence of non-specific control MMAE-conjugated antibody or anti-HU177-MMAE ADC at a concentration of 50 μg/ml over a time course and then analyzed microscopically for evidence of morphological changes that are characteristic of cells beginning to die as a result of disruption of microtubule polymerization. As shown in FIGS. 3A-3C, MCA205 Fibrosarcoma cells, SKOV-3 ovarian carcinoma cells and B16F10 melanoma cells treated over a time course with anti-HU177-MMAE ADC exhibited clear evidence of dramatic changes in cell morphology as compare to MMAE-conjugated control antibody, including cell shrinkage, rounding and the appearance of cell fragmentation (black arrows). Moreover, while treatment of ID8 ovarian carcinoma cells for 72 hours with unconjugated anti-HU177 or non-specific control antibody show little if any change in overall cell morphology following treatment (FIG. 3D), ID8 cells treated with anti-HU177-MMAE ADC at identical concentrations (50 μg/ml) exhibited dramatic cell rounding, shrinkage and fragmentation (black arrows). Collectively, these surprising findings are consistent with the unique ability of the anti-HU177-MMAE ADC, but not the control antibodies, to be selectively internalized where the MMAE can be released to deliver the free MMAE that can result in disruption of microtubules within several malignant tumor cell types.

### Example 4: The Anti-HU177-MMAE ADC Inhibits the Growth of Metastatic B16F10 Melanoma Tumor Cells In Vitro

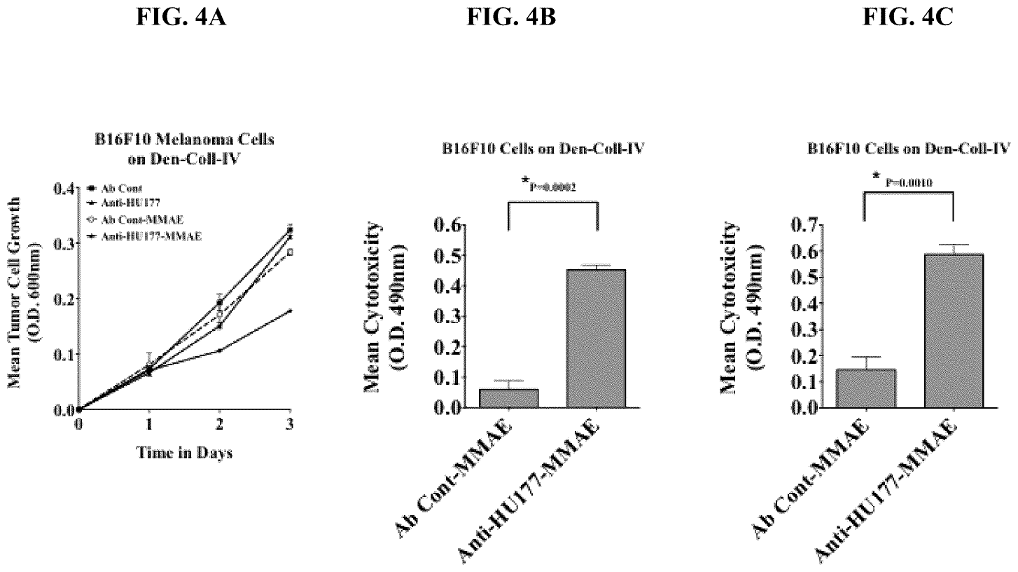

Previous studies have documented that selective targeting of the non-cellular cryptic collagen epitope recognized by anti-HU177 antibody could reduce the growth of different solid tumor types by approximately 35% to 50% in vivo (14, 15, 17-19). Interestingly however, little evidence is available that the unconjugated anti-HU177 antibody could have a growth inhibitory activity directly on tumor cells themselves. In fact, studies have provided evidence that the ability of anti-HU177 to impact solid ovarian tumors in vivo (18), is associated with indirect mechanisms involving disrupting angiogenesis, and the migration of stromal cells by altering unique signaling mechanisms since the antibody is not directed to a cell surface protein (14, 15, 17-19). Given our unexpected observations that the anti-HU177 antibody could be selectively internalized in malignant tumor cells, and that generating an ADC with the cytotoxic drug MMAE did not significantly alter the antibody's functional target binding activity, we sought to determine whether the novel anti-HU177-MMAE ADC, might have direct growth inhibitory activity. To test this possibility, we compared the ability of unconjugated and the MMAE-conjugated anti-HU177 ADC to impact the growth of B16F10 tumor cells. Briefly, serum starved B16F10 melanoma cells were resuspended in serum containing medium in the presence of unconjugated non-specific control antibody, unconjugated anti-HU177 antibody, MMAE-conjugated non-specific control antibody and MMAE-conjugated anti-HU177 antibody and seeded the cells on denatured collagen type-IV coated microtiter wells. Tumor cells were allowed to grow over a 3 day time course. As shown in FIG. 4A, the unconjugated anti-HU177 antibody had little if any ability to inhibit B16F10 melanoma cell growth over a 3 day treatment period as compared to either unconjugated non-specific control antibody or the MMAE-conjugated control antibody (FIG. 4A). Surprisingly, the MMAE-conjugated anti-HU177 antibody significantly inhibited the growth of B16F10 melanoma cells between days 2 and 3 following treatment as compared controls (FIG. 4A). These unexpected findings demonstrate for the first time, that the unique anti-HU177-MMAE antibody conjugate can serve as a critical example of a novel ADC that is not directed to a typical cell surface receptor, that can function to directly inhibit melanoma cell growth in vitro. In further studies, given that MMAE is known to have the capacity to induce cellular cytotoxicity, we examined the ability of the anti-HU177-MMAE ADC to directly induce cytotoxicity in B16F10 melanoma cells using a commercially available cytotoxicity assay (CytoTox 96 Non-radioactive Cytotoxicity Assay). As shown in FIGS. 4B and 4C, the anti-HU177-MMAE ADC at either 50 μg/ml (FIG. 4B) or 100 μg/ml (FIG. 4C) significantly induced B16F10 cell cytotoxicity as compared to the MMAE-conjugated non-specific control antibody (Ab Cont-MMAE). These data demonstrate for the first time that the unique MMAE-conjugated anti-HU177 antibody which is not directed to a cell surface receptor, can induce cytotoxicity in B16F10 tumor cells.

### Example 5: The Anti-HU177-MMAE ADC Inhibits the Growth of Multiple Tumor Cell Types

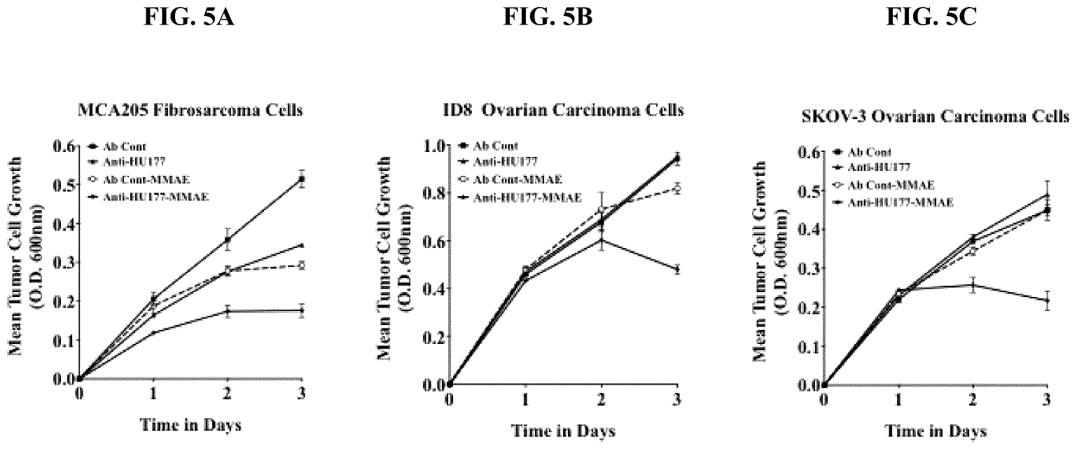

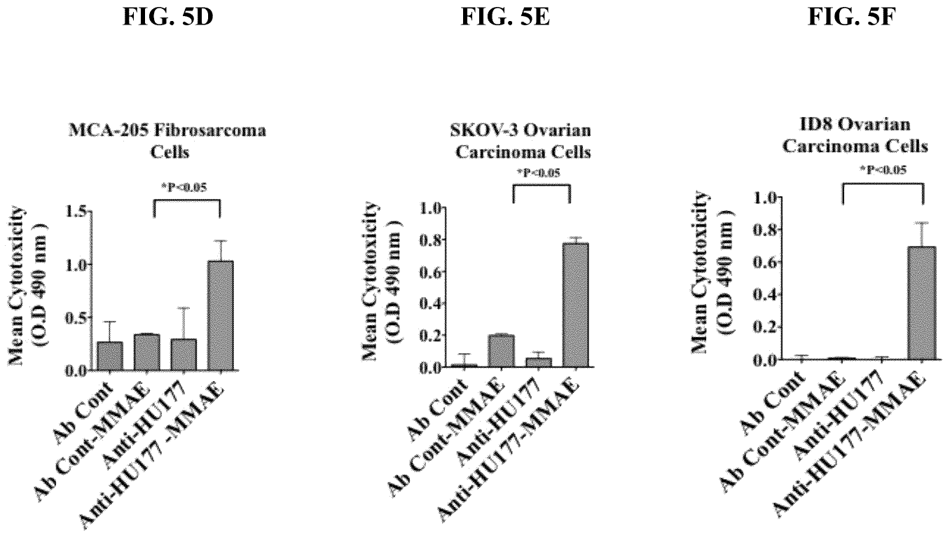

Given the surprising growth inhibitory results achieved with the anti-HU177-MMAE ADC using B16F10 metastatic melanoma cells, we sought to examine the possible effects of this unique anti-HU177-MMAE ADC in other malignant tumor cell types. In this regard, we tested the ability of unconjugated and the MMAE-conjugated anti-HU177 ADC to impact the growth of three different tumor cell types including MCA205 fibrosarcoma cells, ID8 ovarian carcinoma cells and SKOV-3 ovarian carcinoma cells. Briefly, serum starved tumor cells were resuspended in serum containing medium in the presence of unconjugated non-specific control antibody, unconjugated anti-HU177 antibody, MMAE-conjugated non-specific control antibody and MMAE-conjugated anti-HU177 antibody and seeded cells on denatured collagen type-IV coated microtiter wells. Tumor cells were allowed to grow over a 3 day time course. A shown in FIGS. 5A-5C, the anti-HU177-MMAE ADC showed essentially complete inhibition of MCA205 fibrosarcoma, SKOV-3 and ID8 ovarian carcinoma cell growth between days 2 and 3 following treatment. With the exception of MCA205 fibrosarcoma, the unconjugated and MMAE conjugated non-specific control antibody showed minimal if any ability to inhibit tumor cell growth. In further studies, we examined the ability of the anti-HU177-MMAE ADC to directly induce cytotoxicity within these distinct tumor cell types using a commercially available cytotoxicity assay as we described above. As shown in FIGS. 5D-5F, the anti-HU177-MMAE ADC significantly induced tumor cell cytotoxicity as compared to the MMAE-conjugated non-specific control antibody. Collectively, these unexpected and very surprising new data demonstrating that a unique antibody that selectively targets a non-cell surface extracellular cryptic collagen epitope has the capacity to act as a novel ADC with the ability to inhibit the growth of several different tumor cell types.

### Example 6: The Anti-HU177-MMAE ADC Induces Cytotoxicity in Multiple Human Tumor Cell Types

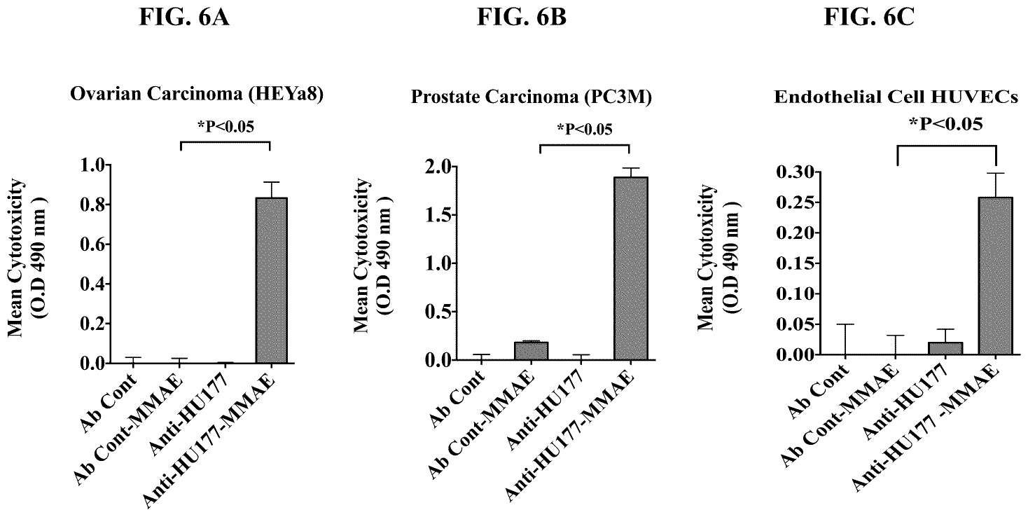

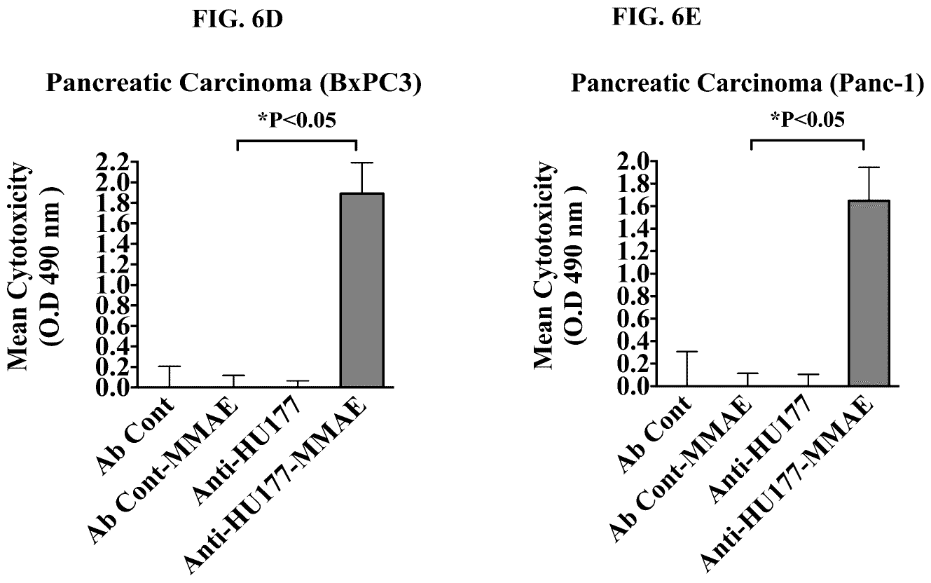

Given the surprising ability of the anti-HU177-MMAE ADC to directly induce cytotoxicity in B16F10 melanoma cells, we next evaluated its ability to induce cytotoxicity in other malignant tumor cell types. To this end, we tested the ability of anti-HU177-MMAE ADC to induce cytotoxicity in 3 human tumor cell types, namely, human HEYa8 ovarian tumor cells, human PC3M prostate tumor cells, and human HUVEC endothelial cells, and compared it with unconjugated non-specific control antibody, unconjugated anti-HU177 antibody, and MMAE-conjugated non-specific control antibody. Briefly, microtiter wells were coated with denatured collagen type-IV, washed and then blocked with BSA to limit non-specific binding. Serum starved tumor cells were resuspended in serum (2.0% FBS for human HEYa8 ovarian tumor and HUVEC endothelial cells; 5.0% FBS for human PC3M prostate tumor cells) containing medium with the indicated unconjugated or MMAE-conjugated antibodies at a concentration of 50 μg/ml (for HEYa8 ovarian and PC3M prostate tumor cells), or 15 μg/ml (for HUVEC endothelial cells) and seeded on the coated wells. Cytotoxicity was quantified at 72 hours. As shown in FIGS. 6A-6C, the anti-HU177-MMAE ADC significantly induced tumor cell cytotoxicity as compared to the MMAE-conjugated non-specific control antibody in all tumor cell types tested. Next, we tested the ability of the anti-HU177-MMAE ADC to induce cytotoxicity in 2 human pancreatic tumor cell types, namely, BxPC3 and Panc-1 cell types. Similar to the findings from other tumor cell types, the anti-HU177-MMAE ADC significantly induced tumor cell cytotoxicity as compared to the MMAE-conjugated non-specific control antibody in both pancreatic tumor cell types, at a concentration of 50 μg/ml (FIGS. 6D-6E). Collectively, these unexpected and surprising new data demonstrate that a unique antibody that selectively targets a non-cell surface extracellular cryptic collagen epitope has the capacity to act as a novel ADC with the ability to inhibit the growth of multiple different tumor cell types.

### Example 7: The Anti-HU177-MMAE ADC Induces Cytotoxicity in Multiple Murine Tumor Cell Types

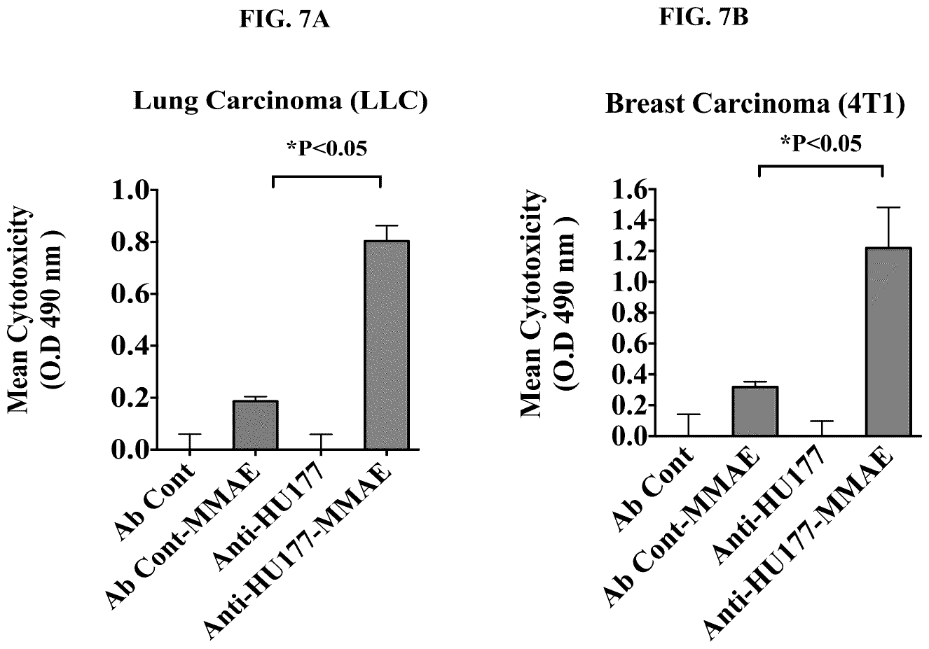

Given the surprising ability of the anti-HU177-MMAE ADC to directly induce cytotoxicity in multiple human tumor cell types, we next evaluated its ability to induce cytotoxicity in malignant murine cells of other tumor cell types. To this end, we tested the ability of anti-HU177-MMAE ADC to induce cytotoxicity in 2 murine tumor cell types, namely, murine LLC lung tumor, and murine 4T1 tumor cells, and compared it with unconjugated non-specific control antibody, unconjugated anti-HU177 antibody, and MMAE-conjugated non-specific control antibody. Briefly, microtiter wells were coated with denatured collagen type-IV, washed and then blocked with BSA to limit non-specific binding. Serum starved tumor cells were resuspended in serum (5.0%) containing medium with the indicated unconjugated or MMAE-conjugated antibodies at a concentration of 50 μg/ml (for LLC lung tumor cells), or 75 μg/ml (for 4T1 breast tumor cells) and seeded on the coated wells. Cytotoxicity was quantified at 72 hours. As shown in FIGS. 7A-7B, the anti-HU177-MMAE ADC significantly induced tumor cell cytotoxicity as compared to the MMAE-conjugated non-specific control antibody in lung and breast carcinoma cell types. Collectively, these unexpected and surprising new data demonstrate that a unique antibody that selectively targets a non-cell surface extracellular cryptic collagen epitope has the capacity to act as a novel ADC with the ability to inhibit the growth of multiple different tumor cell types.

### Example 8: The Anti-HU177-MMAE ADC Inhibits the Growth of Ovarian Tumors In Vivo

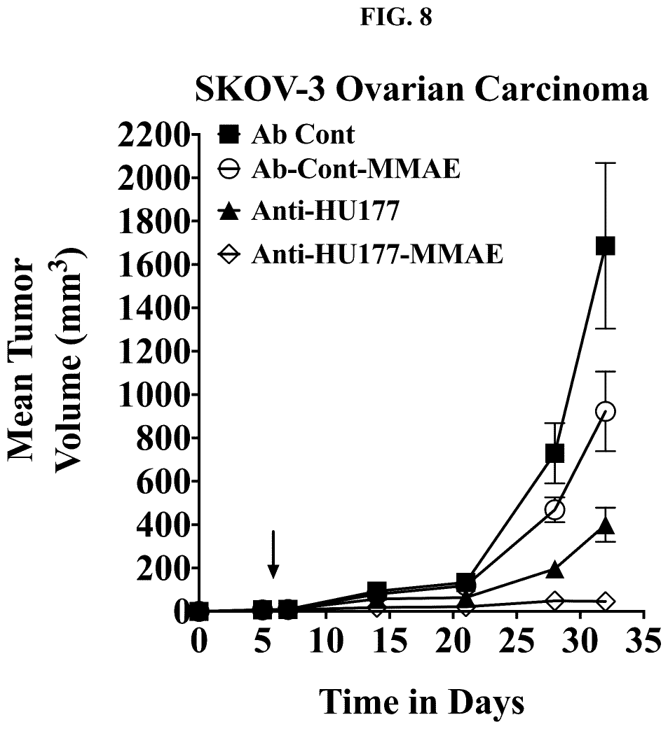

Given the surprising ability of the anti-HU177-MMAE ADC to significantly inhibit tumor cell type growth and enhance cytotoxicity in multiple tumor cell types in vitro, we next examined its activity in inhibiting tumor growth in vivo. To evaluate its therapeutic activity in vivo, mice (N=6 per condition) were injected subcutaneously with human SKOV-3 tumor cells and allowed to establish pre-existing tumors. Beginning on day 6, mice were treated 3× per week with 100 μg per injection of the indicated unconjugated or MMAE conjugated antibodies. Treatment was stopped on Day 21. Mice were sacrificed on Day 32 and all tumors analyzed, and the mean tumor volume quantified. As shown in FIG. 8, administration of the anti-HU177-MMAE ADC beginning on day 6 (black arrow) led to a drastic inhibition of tumor growth, and significantly reduced mean tumor volume at Day 21 compared to either MMAE-conjugated non-specific control antibody, or unconjugated anti-HU177 antibody. These results thus indicate that the Anti-HU177-MMAE ADC enhances the in vivo therapeutic activity of the Anti-HU177 antibody, while serving as a novel ADC with the ability to inhibit tumor growth in vivo.

### Example 9: The Anti-HU177-MMAE ADC Inhibits the Growth of Fibrosarcoma Tumors In Vivo

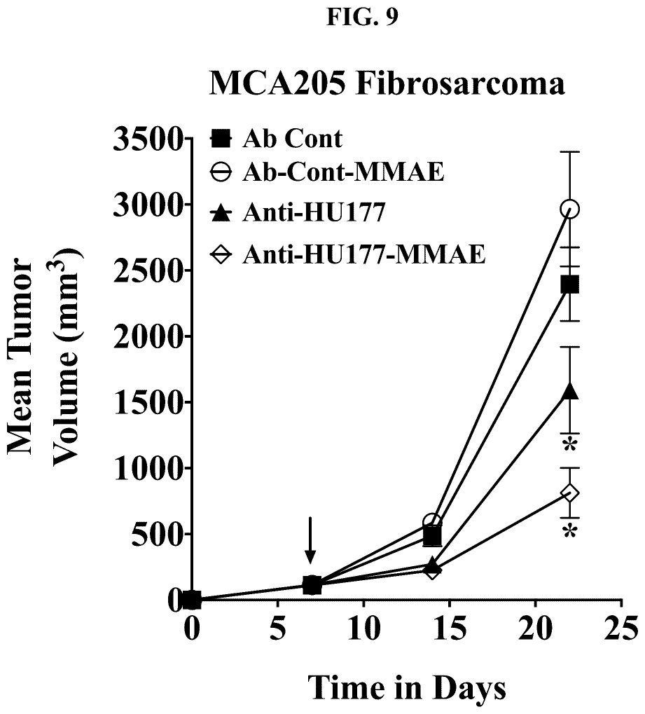

Given the surprising ability of the anti-HU177-MMAE ADC to significantly inhibit tumor growth of ovarian tumors in vivo, we next examined its therapeutic activity in inhibiting the growth of a different tumor type in vivo. To this end, mice (N=6 per condition) were injected subcutaneously with murine MCA205 tumor cells and allowed to establish pre-existing fibrosarcoma tumors. Beginning on day 7, mice were treated 3× per week with 100 μg per injection of the indicated unconjugated or MMAE conjugated antibodies. Mice were sacrificed and all tumors analyzed, and the mean tumor volume quantified. As shown in FIG. 9, administration of the anti-HU177-MMAE ADC beginning on day 7 (black arrow) led to a drastic inhibition of tumor growth, and significantly reduced mean tumor volume at Day 22 compared to either MMAE-conjugated non-specific control antibody, or unconjugated anti-HU177 antibody. These results thus indicate that the Anti-HU177-MMAE ADC enhances the in vivo therapeutic activity of the Anti-HU177 antibody, while serving as a novel ADC with the ability to inhibit the growth of several tumor types in vivo.

### Example 10: An Anti-HU177-MMAF ADC Induces Cytotoxicity in Multiple Tumor Cell Types

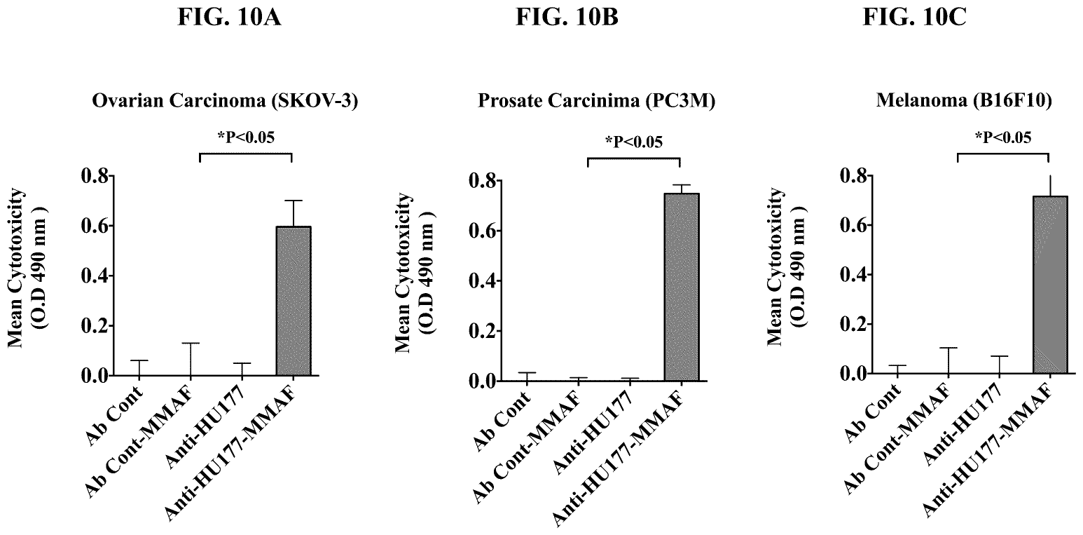

Given our surprising new studies indicating, for the first time, that the novel anti-HU177-MMAE conjugated ADC specifically directed to a cryptic collagen epitope exposed within denatured forms of collagen can be selectively internalized in multiple tumor cell types leading to cytotoxicity and cell death, we next assessed whether this anti-HU177 Mab might represent a unique delivery system for different therapeutic drugs, and not just MMAE. Therefore, as a representative example of the ability of anti-HU177 to deliver different therapeutic drugs to tumor cells, we conjugated monomethyl auristatin F(MMAF) to the anti-HU177 antibody, generating an anti-HU177-MMAF ADC. We then evaluated the ability of the anti-HU177-MMAF ADC to induce cytotoxicity in a number of different tumor cell types including, human ovarian carcinoma cells (SKOV-3), human metastatic prostate carcinoma cells (PC3M), and metastatic mouse melanoma cells (B16F10). To this end, microtiter wells were coated with denatured collagen type-IV, washed and then blocked with BSA to limit non-specific binding. Serum starved tumor cells were resuspended in serum (5.0% FBS) containing medium with the indicated unconjugated or MMAF-conjugated antibodies at a concentration of 50 μg/ml, and seeded on the coated wells. Cytotoxicity was quantified at 72 hours. As shown in FIGS. 10A-10C, the anti-HU177-MMAF ADC significantly induced tumor cell cytotoxicity as compared to the MMAF-conjugated non-specific control antibody in ovarian, prostate and melanoma cell types, respectively. Collectively, these unexpected and surprising new data demonstrate that a unique antibody that selectively targets a non-cell surface extracellular cryptic collagen epitope has the capacity to act as a novel ADC with the ability to deliver multiple therapeutic drugs to tumor cells, thereby inhibiting the growth of multiple different tumor cell types.

### Example 11: Determining Therapeutic Activity of an Anti-XL313-MMAE ADC in Inhibiting of Tumor Growth

In order to evaluate the ability of an anti-XL313 antibody to deliver different therapeutic drugs to tumor cells, monomethyl auristatin E (MMAE) (or monomethyl auristatin F(MMAF)) is conjugated to the anti-XL313 antibody, generating an anti-XL313-MMAE ADC. The ability of the anti-XL313-MMAE ADC to induce cytotoxicity in different tumor cell types is evaluated in vitro. Microtiter wells are coated with denatured collagen type-IV, washed and then blocked with BSA to limit non-specific binding. Serum starved tumor cells are resuspended in serum (5.0% FBS) containing medium with unconjugated or MMAE-conjugated antibodies at a concentration of 50 μg/ml, and seeded on the coated wells. Cytotoxicity is quantified at 72 hours. It is expected that tumor cell cytotoxicity induced by the anti-XL313-MMAE ADC will be higher than a non-specific control antibody, an MMAE-conjugated non-specific control antibody, or an unconjugated anti-XL313 antibody.

In order to evaluate the ability of the anti-XL313-MMAE ADC to inhibit tumor growth in vivo, mice are injected subcutaneously with murine MCA205 or human SKOV-3 tumor cells and allowed to establish pre-existing fibrosarcoma tumors. Beginning on day 7, mice are treated 3× per week with 100 μg per injection of either unconjugated or MMAE conjugated antibodies. Mice are sacrificed and all tumors analyzed, and the mean tumor volume quantified. It is expected that administration of the anti-XL313-MMAE ADC beginning on day 7 will lead to inhibition of tumor growth, and reduction in mean tumor volume compared to either MMAE-conjugated non-specific control antibody, or unconjugated anti-XL313 antibody.

### REFERENCES

1). Wang, M., Li, L., Liu, J., and Wang, J. A gene interaction network-based method to measure the common and heterogeneous mechanisms of gynecological cancer. Mol. Med. Rep. 18:230-242. 2018.
2). Horowitz, M., Esakov, E., Rose, P., and Reizes, O. Signaling within the epithelial ovarian cancer tumor microenvironment: the challenge of tumor heterogeneity. Ann. Tansl. Med. doi: 10.21037/atm-2019-cm-08. 2022.
3). Funingana, I-G., Bedia, J. S., Huang, Y-W., Gonzalez, A. D., Donoso, K., Gonzalez, V. D., Brenton, J. D., Ashworth, A., and Fantl, W. J. Multiparameter single-cell proteomic technologies give new insight into the biology of ovarian tumors. Semin. Immunopathol. 45:43-59. 2023.
4). Chandra, A., Pius, C., Nabeel, M., Nair, M., Vishwanatha, J. K., Ahmad, S., Basha, R. Ovarian cancer: current status and strategies for improving therapeutic outcomes. Cancer Med. 16:7018-7031. 2019.
5). Aliyuda, F., Moschetta, M., Ghose, A., Rallis, K. S., Sheriff, M., Sanchez, E., Rassy, E., and Boussios, S. Advances in ovarian cancer treatment beyond PARP inhibitors. Curr. Cancer Drug Targets. doi: 10.2174/15680096236623029121732. 2023.
6). Hu, X., Bian, C., Zhao, X., and Yi, T. Efficacy evaluation of multi-immunotherapy in ovarian cancer: from bench to bed. Front Immunol. doi: 10.3389/fimmu.2022.1034903. 2022.
7). Yoon, H., Kim, A., and Jang, H. Immunotherapeutic approaches in ovarian cancer. Curr. Issues Mol. Biol. 45:1233-1249. 2023.
8). Hollis, R. L. Molecular characteristics and clinical behavior of epithelial ovarian cancer. Cancer Lett. doi: 10.1016/j.canlet.2023.216057. 2023.
9). Buklaho, P. A., Kisluk, J., Wasilewska, N., and Niklinski, J. Molecular features as promising biomarkers in ovarian cancer. Adv Clin. Exp. Med. doi: 10.17219/acem/159799. 2023.
10). Santais, M., Simojoki, M., Risteli, J., Risteli, L., and Kauppila A. Type I and III collagen metabolites as predictors of clinical outcomes in epithelial ovarian cancer. Clin. Cancer Res. 12:4091-4096. 1999.
11). Natarajan, S., Foreman, K. M., Soriano,M., Rossen, N. S., Shehade, H., Fregoso, D., R., Eggold, J. T., Krishnan, V., Dorigo, O., Krieg, A., Heilshorn, S. C., Sinha, S., Fuh, K. C., and Rankin, E. B. Collagen remodeling in the hypoxic tumor-mesothelial niche promotes ovarian cancer metastasis. Cancer Res. 79:2271-2284. 2019.
12). Zhang, J., Zhang, J., Wang, F., Xu, X., Li, X., Guan, W., Men, T., and Xu, G. Overexpressed Col5A is correlated with tumor progression, paclitaxel resistance, and tumor infiltrating immune cells in ovarian cancer. J. Cell. Physiol. 236:6907-6919. 2021.
13). Pickup, M. W., Mouw, J. K., and Weaver, V. M. The extracellular matrix modulates the hallmarks of cancer. EMBO Reports. 15:1243-1253. 2014.
14). Pernasetti, F., Nickel, J., Clark, D., Baeuerle, P. A., Van Epps, D., and Freimark, B. Novel anti-denatured collagen humanized antibody D93 inhibits angiogenesis and tumor growth: an extracellular matrix-based therapeutic approach. Int. J. Oncol. 29:1371-1379. 2006.
15). Freimark, B., Clark, D., Pernasetti, F., Nickel, J., Clark, D., Myszka, D., Baeuerle, P. A., and Van Epps, D. Targeting of humanized antibody D93 to sites of angiogenesis and tumor growth by binding to multiple epitopes on denatured collagens. Mol. Immunol. 44:3741-3750. 2007.
16). Robert, F, Gordon, MS, Rosen, LS, Mendelson, DS, Mulay, M, Adams, BJ, Alvare, D, Theuer, CP, Leigh, BR: Final results from a phase I study of TRC093 (humanized anti-cleaved collagen antibody) in patients with solid cancer. Annual meeting proceedings, 2010, ASCO. Abstract No 3038. 2010.
17). Xu, J., Rodrigeuze, D., Kim, J., and Brooks, P. C. Generation of monoclonal antibodies to cryptic collagen sites by subtractive immunization. Hybridoma. 19:375-385. (2000).
18). Caron, J. M., Ames, J. J., Contois, L., Liebes, L., Friesel, R. E., Vary, C. P. H., Oxburg, L., and Brooks, P. C. Inhibition of ovarian tumor growth by targeting the HU177 cryptic collagen epitope. AM. J. Pathol: 2016; 186:1649-1661.
19). Caron, J. M., Xianghu, H., Contois, L., Vary, C. P. H., and Brooks, P.C. The HU177 collagen epitope controls melanoma cell migration and experimental metastasis by a CDK5/YAP-dependent mechanism. AM. J. Pathol. pii: S0002-9440 (18) 30253-0. doi: 10.1016/j.ajpath.2018.06.017. PMID: 30118657. 2018.
20). Ames, J. J., Contois, L., Caron, J. M., Tweedie, E., Yang, X., Friesel, R., Vary, C., and Brooks, P. C. Identification of an endogenously generated cryptic collagen epitope (XL313) that may selective regulates angiogenesis by an integrin-Yes-associated protein (YAP) mechano-transduction pathway. J. Biol. Chem. 2016 Feb. 5; 291 (6): 2731-2750.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

1. An antibody-drug conjugate (ADC) comprising an antibody, or an antigen binding fragment thereof, conjugated to at least one drug, and
wherein the antibody, or the antigen binding fragment thereof, binds to collagen and is internalized in a cancer cell.

2. The ADC of claim 1, wherein the collagen comprises collagen type-I, collagen type II, collagen type III, or collagen type-IV.

3. (canceled)

4. The ADC of claim 1, wherein the collagen is denatured.

5. The ADC of claim 1, wherein:
the antibody, or the antigen binding fragment thereof, binds a cryptic collagen epitope comprising one or more amino acid sequences selected from the group consisting of GPOG (SEQ ID NO: 4), GPOGPOP (SEQ ID NO: 5), GPPG (SEQ ID NO: 6), and GPPGPPG (SEQ ID NO: 7); and/or
the antibody, or the antigen binding fragment thereof, binds a cryptic collagen epitope comprising one or more amino acid sequences selected from the group consisting of CPGFPGFC (SEQ ID NO: 16), PGAKGLPGPPGPPGPY (SEQ ID NO: 17), GFOGIOGTOGPOGLO (SEQ ID NO: 18), GEXGDOGIAGFOGSO (SEQ ID NO: 19), GPQGQPGLOGLOGPM (SEQ ID NO: 20), GFOGIOT (SEQ ID NO: 21), and GDTGPOGPOGY (SEQ ID NO: 22).

6. (canceled)

7. The ADC of claim 1, wherein the antibody, or the antigen binding fragment thereof, binds a cryptic collagen epitope comprising an amino acid sequence that has at least 90% identity to PGAKGLPGPPGPPGPY (SEQ ID NO: 17).

8. The ADC of claim 1, wherein the antibody comprises a monoclonal antibody.

9. The ADC of claim 8, wherein said monoclonal antibody comprises an HU177 monoclonal antibody deposited with ATCC accession number PTA-6551.

10. The ADC of claim 1, wherein the ADC is not directed to a receptor on the cancer cell surface.

11. The ADC of claim 1, wherein the at least one drug is selected from the group consisting of an apoptotic agent, a mitotic inhibitor, an anti-tumor antibiotic, an immunomodulating agent, a nucleic acid for gene therapy, an anti-angiogenic agent, an anti-metabolite, a boron-containing agent, a chemoprotective agent, a hormone agent, an anti-hormone agent, a DNA damaging agent, a corticosteroid, a photoactive therapeutic agent, an oligonucleotide, a radionuclide agent, a radiosensitizer, a topoisomerase inhibitor, and a tyrosine kinase inhibitor.

12. The ADC of claim 11, wherein:
the mitotic inhibitor is selected from the group consisting of monomethyl auristatin E (MMAE), monomethyl auristatin F(MMAF), mertansine (DM1), and ravtansine (DM4); and/or
the DNA damaging agent is selected from the group consisting of psilocybin, gliclazomycin, streptomycin, pyrrolobenzodiazepine (PBD), doxorubicin, and adrianmycin.

13-15. (canceled)

16. The ADC of claim 1, wherein the at least one drug is conjugated to the antibody, or the antigen-binding portion thereof, via a linker, wherein the linker is a cleavable linker or a non-cleavable linker.

17. (canceled)

18. A pharmaceutical composition comprising the ADC of claim 1, and a pharmaceutically acceptable carrier.

19. A method of treating cancer in a subject, the method comprising administering to the subject a therapeutically effective amount of the ADC of claim 1, thereby treating the cancer.

20. A method for inhibiting or decreasing growth of a solid tumor in a subject having a solid tumor, the method comprising administering to the subject a therapeutically effective amount of the ADC of claim 1, thereby inhibiting or decreasing the growth of the solid tumor in the subject.

21-22. (canceled)

23. The method of claim 20, wherein:
the mean tumor volume in the subject who has been administered the ADC is at least 1.1 fold, at least 1.5 fold, at least 2 fold, at least 5 fold, at least 10 fold, at least 20 fold, at least 50 fold, at least 100 fold, at least 200 fold lower compared to a control;
the growth rate of the tumor in the subject who has been administered the ADC is at least 1.1 fold, at least 1.5 fold, at least 2 fold, at least 5 fold, at least 10 fold, at least 20 fold, at least 50 fold, at least 100 fold, at least 200 fold lower compared to a control; and/or
cell death in the tumor in the subject who has been administered the ADC is at least 1.1 fold, at least 1.5 fold, at least 2 fold, at least 5 fold, at least 10 fold, at least 20 fold, at least 50 fold, at least 100 fold, at least 200 fold higher compared to a control;
wherein the control is prior to administration of the ADC.

24-28. (canceled)

29. A method of delivering a drug to a cancer cell in a subject, the method comprising administering to the subject a therapeutically effective amount of the ADC of claim 1.

30-31. (canceled)

32. The method of claim 19, further comprising administering concurrent or sequential radiotherapy, monoclonal antibodies, chemotherapy, immunotherapy or other anticancer drugs or interventions to the subject.

33. The method of claim 32, wherein the immunotherapy comprises administering an immune checkpoint inhibitor, wherein the immune checkpoint inhibitor comprises an inhibitor of CTLA-4, PD-1, PDL-1, Lag3, LAIR1, or LAIR2.

34-36. (canceled)

37. The method of claim 19, the method further comprising inhibiting an inflammatory condition, wherein said inflammatory condition is selected from the group consisting of an allergy, ankylosing spondylitis, asthma, atopic dermatitis, an autoimmune disease or disorder, a cancer, celiac disease, chronic obstructive pulmonary disease (COPD), chronic peptic ulcer, cystic fibrosis, diabetes, glomerulonephritis, gout, hepatitis, an immune-mediated disease or disorder, inflammatory bowel disease (IBD), myositis, osteoarthritis, pelvic inflammatory disease (PID), multiple sclerosis, neurodegenerative diseases of aging, a periodontal disease, reperfusion injury transplant rejection, psoriasis, pulmonary fibrosis, rheumatic disease, scleroderma, sinusitis, dermatitis, pneumonitis, colitis and tuberculosis.

38. The method of claim 19, wherein the cancer is selected from the group comprising of melanoma, central nervous system (CNS) cancer, CNS germ cell tumor, lung cancer, a leukemia, a lymphoma, multiple myeloma, renal cancer, malignant glioma, medulloblastoma, breast cancer, ovarian cancer, prostate cancer, bladder cancer, fibrosarcoma, pancreatic cancer, gastric cancer, head and neck cancer, colorectal cancer, a cancer cell derived from a solid cancer, or a hematological cancer;
wherein the leukemia is acute lymphoblastic leukemia (ALL), acute myelogenous leukemia (AML), chronic lymphocytic leukemia (CLL), small lymphocytic lymphoma (SLL), chronic myelogenous leukemia (CML) or acute monocytic leukemia (AMOL),
wherein the lymphoma is follicular lymphoma, Hodgkin's lymphoma, or Non-Hodgkin's lymphoma, wherein the Hodgkin's lymphoma is Nodular sclerosing subtype, mixed-cellularity subtype, lymphocyte-rich subtype, or lymphocyte depleted subtype.

39-48. (canceled)
